(19) United States
(12) Patent Application Publication
(10) Pub. No.: US 2021/0017607 A1
(43) Pub. Date: Jan. 21, 2021

(54) Methods of Predicting Responsiveness to Cancer Therapies

(71) Applicant: The University of Chicago, Chicago, IL (US)

(72) Inventors: Akash Patnaik, Chicago, IL (US); Priyanka Dutta Gupta, Chicago, IL (US)

(21) Appl. No.: 16/933,885

(22) Filed: Jul. 20, 2020

Related U.S. Application Data
(60) Provisional application No. 62/875,966, filed on Jul. 18, 2019.

(51) Int. Cl. C12Q 1/6886; C12Q 1/6869

(52) U.S. Cl. CPC C12Q 1/6886; C12Q 1/6869

(57) ABSTRACT

The present disclosure relates to biomarkers for and methods of treating cancer.

### STATEMENT REGARDING FEDERALLY SPONSORED RESEARCH OR DEVELOPMENT

This invention was made with government support under grant number P50CA180995 awarded by the National Institutes of Health. The government has certain rights in the invention.

### BACKGROUND OF DISCLOSURE

### Field of Invention

This disclosure relates to biomarkers and methods of treatment for cancer.

### Technical Background

### Cancer Biomarkers

A biomarker is an objectively measured characteristic that describes a normal or abnormal biological state in an organism that can be identified by analyzing biomolecules such as DNA, RNA, protein, peptide, and biomolecule chemical modifications associated with the normal or abnormal biological state. The World Health Organization broadly defines the term as follows: “A biomarker is any substance, structure or process that can be measured in the body or its products and influence or predict the incidence of outcome or disease.” More specifically in terms of clinical utility, a cancer biomarker may measure the risk of developing cancer in a specific tissue or, alternatively, may measure risk or degree of cancer progression or potential response to therapy. Besides providing useful information for guiding clinical decision making, cancer biomarkers are increasingly linked to specific molecular pathway deregulation and/or cancer pathogenesis to justify application of certain therapeutic/interventional strategies.

Cancer biomarkers can be classified into various categories based on their usage. Predictive biomarkers predict response to specific therapeutic interventions. Prognostic biomarkers, on the other hand, may not be directly linked to or trigger specific therapeutic decisions, but can be used to inform physicians regarding the risk of clinical outcomes such as cancer recurrence or disease progression in the future. Another class of biomarker, the diagnostic biomarker, is used to identify whether a patient has a specific disease condition.

### Types of Cancer Therapies in Need of Relevant Biomarkers

PI3K inhibitors. Phosphoinositide 3-kinases (PI3K) are involved in multiple cellular processes including metabolism, motility, proliferation, growth, and survival, and are the most frequently dysregulated pathways in human cancers. More than 40 inhibitors of the PI3K signaling pathway have reached different stages of clinical development, but few—including temsirolimus, everolimus, idelalisib, and copanlisib—have been approved for clinical use. Both limited single-agent activity and lack of predictive biomarkers have plagued the development of therapies for this promising pathway.

PARP inhibitors. Poly (ADP-ribose) polymerase (PARP) is an important protein in DNA repair pathways. In cancer treatment, blocking PARP can prevent cancer cells from repairing their damaged DNA, causing them to die. PARP inhibitors have been of interest in both single-agent regimens as well as combination therapy approaches.

STING agonists. Agonists of stimulator of interferon genes (STING), a receptor that triggers an immune response when stimulated by pathogen DNA, have recently attracted interest as cancer immunotherapies, with several agents now in clinical trials.

Immune checkpoint blockade. Checkpoint inhibitor therapy, or immune checkpoint blockade, is another treatment paradigm for cancer that leverages the body's immune system to achieve a therapeutic effect. Checkpoint inhibitors prevent checkpoint proteins (e.g., PD-1) from binding to their partner proteins or ligands (e.g., PD-L1) and thereby reverse an “off” switch mechanism that prevents immune cells from attacking cancer cells. Investigations of immune checkpoint inhibitors, such as PD-1 inhibitors have primarily focused on enhancing T-cell function, in part, through increased type II IFN production. While a number of these inhibitors have shown great clinical promise, the percentage of patients estimated to respond to currently available checkpoint inhibitor drugs was only 12.46% in 2018.

In light of the challenges in development and low percentages of successful outcomes for some of these therapeutic approaches, means for assessing patients before administration of therapies are becoming increasingly critical. There is a need for clinically useful biomarkers, particularly predictive biomarkers, both in developing new cancer therapies and in selecting patient treatment regimens.

### SUMMARY OF THE DISCLOSURE

This disclosure describes methods of using biomarkers to improve outcomes in the treatment of cancer.

As described below, in one aspect, the disclosure provides a method of treating cancer in a subject, comprising

determining a PTEN status of a tumor of the subject; and

administering a PARP inhibitor and a PI3Kinase inhibitor to the subject if the status of the tumor is determined to be PTEN-proficient.

In one embodiment of the first aspect, the cancer is prostate cancer. In another embodiment, the method further includes administering androgen deprivation therapy to the subject. In another embodiment, determining the PTEN status comprises taking a tissue biopsy from the tumor of the subject. In another embodiment, determining the PTEN status comprises next generation sequencing and/or immunohistochemistry. In another embodiment, the method further includes administering an immune checkpoint blockade-targeting therapy to the subject.

In a second aspect, the disclosure provides a method of treating cancer in a subject, comprising

determining a PTEN status of a tumor of the subject; and

administering a STING agonist to the subject if the status of the tumor is determined to be PTEN-deficient,

wherein the tumor is PTEN-deficient if one or more cells within the tumor is determined to be PTEN-deficient.

In one embodiment of the second aspect, the tumor is PTEN-deficient if one or more cells within the tumor is determined to be PTEN-deficient. In another embodiment, the cancer is prostate cancer. In a further embodiment, the method further includes administering androgen deprivation therapy to the subject. In another embodiment, the subject is non-responsive to androgen deprivation therapy. In a further embodiment of the second aspect, the cancer is ovarian cancer, such as advanced ovarian cancer. In a further embodiment of the second aspect, the cancer is endometrial cancer. In one embodiment, the method can further include administering chemotherapy to the subject. In another embodiment, determining the PTEN status includes taking a tissue biopsy from the tumor of the subject. In another embodiment, determining the PTEN status comprises employing next generation sequencing and/or immunohistochemistry. In some embodiments, the STING agonist is BMS-986301.

In another embodiment, the method can further include administering a PI3Kinase inhibitor to the subject.

In a third aspect, the disclosure provides a method of treating cancer in a subject, comprising

detecting the presence of DNA double-strand break fragments associated with an exosome or a microvesicle in a tumor microenvironment; and

administering an immune checkpoint blockade-targeting therapy and a PI3Kinase inhibitor to the subject if DNA double-strand break fragments associated with an exosome or a microvesicle in the tumor microenvironment are detected.

In one embodiment of the third aspect, detecting the presence of DNA double-strand break fragments comprises taking a blood sample from the subject.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure. For all figures: *p<0.05; **p<0.01; ***p<0.001; ****p<0.0001 between indicated groups or compared to control or as otherwise indicated per figure; ns=not statistically significant.

FIGS. 1A-1C. Most primary and metastatic prostate tumors lack a T cell-inflamed gene signature. (1A) Gene expression from The Cancer Genome Atlast (TCGA) & SU2C databases; (1B) Transparent Tissue Tomography and scRNASeq analysis (1C) of metastatic lymph node biopsy from metastatic castration-resistant prostate cancer (mCRPC) patient progessing on rucaparib/nivolumab.

FIGS. 2A-2D. Low stimulator of interferon genes (STING) expression and myeloid activation markers within tumor-associated macrophages (TAMs) correlate with reduced survival in primary prostate cancer (PC). (2A) cGAS and STING expression; (2B) Immune profiling of metastatic bone marrow aspirate following progression on ipilumumab/nivolumab showing dominant presence of myeloid cells (MDSCs); (2C) BCR curves for Low and High PCM groups myeloid activation (2D) relative gene expression of activated genes.

FIGS. 3A-3C. Additive DNA Damage in Myc-driven PC cells treated with pan-phosphoinositide 3-kinase inhibitor/poly(ADP-ribose) polymerase inhibitor (PI3Ki/PARPi) combination. (3A) Schema showing double-stranded DNA (dsDNA) based activation of STING pathway; (3B) cyclic GMP-AMP synthase (c-GAS)/STING pathway expression in cancer cell lines. Western blotting was performed on protein extracts from Myc-CAP (STING^{lo}) and B6-myc (STING^{hi}) cells, with the indicated c-GAS/STING pathway protein markers; (3C) Quantification of dsDNA/cell within cancer cells.

FIGS. 4A-4D. PARPi/PI3Ki/Androgen deprivation therapy (ADT) Induces Tumor Regression in Myc-CAP via a Non-Tumor Cell Autonomous Immune Mechanism. Syngeneic phosphatase and tensin homolog (PTEN)-proficient Myc-CAP tumor-bearing immuno-competent FVB mice (4A); immuno-deficient mice (4B); castrated immuno-competent mice without anti-PD-L1 antibody (4C); and castrated immuno-competent mice with anti-PDL1 antibody (4D) were treated with the drugs indicated. Measurements were collected until tumor volumes reached 2000-2500 mm³. For all groups, n=3-5 mice.

FIGS. 5A and 5B. The combination of PARPi/PI3Ki/ADT results in increased macrophage activation and CD8 T cell infiltration within Myc-CAP tumors, which is enhanced by immune checkpoint blockade (ICB). Relative mean fluorescence intensity (MFI) for major histocompatibility complex II (MHC-II) on macrophages (CD45+CD11b+F480+) (5A) and % CD8 T cells (5B) in Myc-CAP tumors of uncastrated mice and chemically castrated (via degarelix) mice (5C and 5D, respectively) for indicated treatment groups.

FIGS. 6A and 6B. PARPi/PI3Ki-induced macrophage activation is dependent on STING. (6A) Tumor growth curves for B6MYC in STING+/+ & STING−/− mice; (6B) interferon beta (IFNB1) secretion by STING+/+ & STING−/− bone marow-derived macrophages (BMDM) co-cultured with drug treated tumor cells.

FIGS. 7A-7D. DNA damage-induced STING pathway activation within tumor-associated macrophages is mediated via microvesicles or exosomes carrying dsDNA. (7A) shown left to right: Untreated, DMXAA, Rucaparib, BKM120, and Rucaparib+BKM120, IFNB1 secretion in the presence & absence of GW4869; (7B) shown left to right: Untreated, Rucaparib, BKM120, and Rucaparib+BKM120, Exosome quantification; (7C) Exosomal dsDNA concentration, shown left to right: Untreated, Rucaparib, BKM120, Rucaparib+BKM120; (7D) IFNB1 expression (pg/mL) & Western blot analysis of TBK1 expression in BMDMs with indicated drugs −/+ exosomes released from PTEN-proficient Myc-CAP cells.

FIGS. 8A-8C. PTEN-deficient cancers are resistant to DNA-damage induced STING pathway activation. (8A) shown clockwise from twelve o'olock: CD4, CD8+, CD19+, F4/80+, Mo-MDSC+, Gr-MDSC+, Neutrophil+, DC+, immune profile of wild-type vs. PTEN-deficient cancers; (8B) Diminished IFNB1 secretion in ex vivo reconstituted tumors; (8C) Tumor volume growth in PTEN-deficient vs. PTEN-proficient cancers following treatment with the indicated drugs.

FIGS. 9A and 9B. The combination of PARPi/PI3Ki/ADT fails to enhance macrophage infiltration/activation and CD8/CD4 T cell infiltration within Myc-CAP PTEN deficient tumors. Tumors were harvested from Myc-CAP PTEN−/− syngeneic mice following 20 days of treatment with the indicated drugs, and processed into single cell suspensions for flow cytometry analysis. (9A) Frequency of CD11b+F480+ macrophages and xpression of MHC-II (MFI) on macrophages; (9B) % CD4 and CD8 T cells within corresponding tumors, as assessed by flow cytometry.

FIGS. 10A and 10B. Exosomes released from PTEN-deficient cancer cells have significantly reduced dsDNA content. (10A) Nanoparticle Tracking Analysis (NTA) measurements of exosomes isolated from drug treated tumor supernatants (particles/mL); (10B) measurement of dsDNA cargo (ng/μL) within exosomes following treatment with the indicated drugs.

FIGS. 11A and 11B. A Phase Ib/II Investigator-Initiated Trial of Rucaparib plus Nivolumab in mCRPC patients. (11A) Study design; (11B) Immune profiling of metastatic biopsies showing lymphocytic, myeloid, and granulocytic cell frequencies pre- and post-treatment (left) and ratios of 4-1BB (CD137)/PD-1 expression (MFI) at 0 and 4 weeks.

FIG. 12. Working Model for c-GAS/STING pathway activation induced by PARPi/PI3Ki combination or STING agonists within the tumor microenvironment in prostate cancer (PC). Without wishing to be bound by theory, the combination of PARPi+PI3Ki induces production of DNA double-stranded fragments within cancer cells, which are released into microvesicles that can secondarily activate the c-GAS/STING pathway within myeloid suppressive cells, resulting in myeloid reprogramming and activation. STING pathway activation induced by DNA damaging agents is abolished within the tumor microenvironment (TME) of PTEN-deficient PC. In addition, PI3Ki can inhibit Ser-291 phosphorylation of c-GAS, thus derepressing its enzymatic activity. In response to DNA damage, dsDNA-containing microvesicles can activate the c-GAS/STING pathway within myeloid suppressive cells, resulting in increased T cell infiltration. In contrast, microvesicles released from PTEN-deficient cancer cells lack of dsDNA fragments, resulting in resistance to DNA sensing STING pathway activation, and an absence of an anti-tumor immune response.

FIGS. 13A-13L. The immune infiltrates within human mCRPC and murine c-myc-driven PC tumors are dominated by tumor-associated macrophages (TAMs). Single cell suspensions from human mCRPC biopsies, murine Myc-CAP and B6-Myc syngeneic tumors were stained with anti-human/mouse lineage-specific antibodies and analyzed by flow cytometry for relative immune infiltrates. (A,E,I) Tumor cells (CD45−) vs. Immune cells (CD45+); (B,F,J) Lymphoid—T (CD3+)+B (CD19+) cells vs. Myeloid (CD11b+; macrophages, DCs, MDSCs—monocytic and granulocytic) cells. (C,G,K) Lymphoid and Myeloid compartments were analyzed for individual immune cell subsets using the following additional markers: Macrophages (hCD45+CD11b+CD163+CD68+/mCD45+CD11b+F480+), DCs (hCD45+CD11b+CD11c+/mCD45+CD11b+CD11c+), Gr-MDSCs (hCD45+CD11b+HLA-DR-CD15^{hi}/CD33^{lo}/mCD45+CD11b+MHC-II-Ly6G^{hi}Ly6C^{lo}), Mo-MDSCs (hCD45+CD11b+HLA-DR-CD15^{hi}/CD33^{lo}/mCD45+CD11b+MHC-II-Ly6G^{lo}Ly6C^{hi}); (D,H,L) HLA-DR expression on TAMs in mCRPC & MHC-II expression on TAMs within TME of murine tumors were analyzed; n=4 for human and n=3-4 for murine samples. DC=dendritic cells; MDSC=myeloid derived suppressor cell, h=human, m=mouse.

FIGS. 14A-14H. High-risk PC patients exhibit reduced STING, myeloid activation and T cell chemotactic factor gene expression. (14A-14B) cGAS and STING expression in normal vs. PC within the The Cancer Genome Atlast (TCGA). (14C) PC patients in TCGA were further subdivided into Low and High Gleason score groups, defined as follows: Low Gleason score=6/7 and High Gleason Score>8. Frequency of biochemical recurrence (BCR) for evaluable cases within cohorts was determined and differential gene expression across the two groups was further analyzed for STING (14D), myeloid activation-specific genes (14E-14F), and T cell chemotactic factors (14G-14H) secreted downstream of STING activation; n=290 (Low Gleason group n=211; High Gleason group n=79); Significance/p-values were calculated using Mann Whitney/Un-paired t test for panels (14A-14B, 14D-14H) and Kolmogorov Smirnov test for panel (C).

FIGS. 15A-15E. Differential STING pathway activation within B6-Myc (STING^{hi}) cell lines and BMDMs relative to Myc-CAP (STING^{lo}) cells. (15A, 15B) The indicated cancer cell lines and BMDMs were treated with DMXAA (mouse STING agonist) for 1 and 24 hours, respectively, and protein extracts were interrogated for cGAS/STING pathway activation with the indicated antibodies by Western blotting. Supernatants were collected from for IFNβ1 ELISA following 36 hours of treatment with DMXAA (15C, 15D). (15E) Syngeneic Myc-CAP (STING^{lo}) tumors were processed into single cell suspensions and used for phycoerythrin-based sorting of CD45+ cellular fractions. Equal numbers of cells from these fractions were stimulated with DMXAA. Supernatants were collected at 36 hrs for detection of IFNB1 by ELISA; n=3 independent experiments; Significance/p-values were calculated by one-way Anova (for 15C & 15E) or paired t-test (for 15D); TAM=tumor associated macrophage; s.e.=short exposure; l.e.=long exposure.

FIGS. 16A-16B. PARPi/PD-1 targeted combination therapy shows lack of efficacy in PC co-clinical trials, which can be reversed by concomitant PI3Ki treatment. (16A) mCRPC patients were enrolled in a Phase Ib rucaparib/nivolumab co-clinical trial at University of Chicago, and treated until disease progression or unacceptable toxicity. As part of the study, blood was processed to collect sera for ELISA based determination of PSA (prostate-specific antigen) levels at baseline and every month on study. Data obtained was used to calculate percent change in PSA levels at approximately 90 days, relative to baseline values. Each bar represents a single patient. # indicates patient who progressed early at 41 days, as per RECIST guidelines; n=7 patients. (16B) B6-Myc (STING) tumor-bearing syngeneic mice were treated with the indicated drug(s) and euthanized when untreated tumors reached approx. 2500 mm³. Tumor volume curves for duration of treatment are shown. n=3 animals per treatment group from 2 independent experiments. Significance/p-values were calculated by one-way ANOVA/paired t-test.

FIGS. 17A-17C. PARPi/PI3Ki combination induces intracellular DNA DSBs, and proportionate release of MV surface-associated DNA DSBs from B6-Myc (STING^{hi}) cancer cells, without affecting cellular viability. B6-Myc (STING^{hi}) cells were treated with indicated drugs singly or in combination for 36 hours. (17A) Cells were stained with anti-mouse specific p-γH2AX antibody and fluorescently labeled secondary antibody for determination of DNA DSBs, which were quantified by confocal microscopy. (17B) Protein extracts from cells in (17A) were analyzed for the indicated pharmacodynamic biomarkers by western blotting. (17C) Annexin V-PI staining was done to assess % live cells (Annexin V⁻ PI⁻) following drug treatment. Significance/p-values were calculated by one-way ANOVA.

FIGS. 18A and 18B. Buparlisib and Rucaparib inhibit intratumoral Pi3K and PARP enzymatic activity, respectively, within B6-Myc (STING^{hi}) and Myc-CAP (STING^{lo}) tumors. Subcutaneous Myc-CAP (STING^{lo}) and B6-Myc (STING^{hi}) tumors at 400-500 mm³ volumes were treated with either buparlisib (pan-PI3K inhibitor, 30 mg/kg) or rucaparib (PARP inhibitor, 150 mg/kg) by oral gavage daily for 7 days. At end of treatment, both (18A) B6-Myc (STING^{hi}) and (18B) Myc-CAP (STING^{lo}) tumors were harvested and protein extracts utilized for assessment of target inhibition with the indicated antibodies by western blotting. n=2 mice per treatment group.

FIGS. 19A-19G. PARPi/PD-1 targeted combination therapy shows lack of efficacy in PC co-clinical trials, which can be reversed by concomitant PI3Ki treatment; PARPi/PI3Ki/PD-L1 treatment increases CD4 T cell infiltration within the TME of STING^{hi} B6-Myc tumors. (19A) B6-Myc (STING^{hi}) tumor-bearing syngeneic mice were treated with the indicated drug(s) and euthanized when untreated tumors reached approx. 2500 mm³. Tumor volume curves for duration of treatment are shown. (19B-19G) Single-cell suspensions were generated from harvested tumors and analyzed by flow cytometry for the indicated immune cell populations and their activation states. Data are represented relative to CD45+ immune cells. Animals per treatment group n=3-5 from 2 independent experiments; Significance/p-values were calculated by one-way ANOVA/paired t-test

FIG. 20. The anti-tumor response elicited by PARPi/PI3K is abrogated in B6-Myc (STING^{hi})-tumor bearing athymic nude mice. B6-Myc (STING^{hi}) tumor-bearing athymic nude mice were treated with the indicated drug(s) and tumor growth was measured for approximately 12 days. Tumor volume curves for duration of treatment are shown; n=3 animals/group; Significance/p-values were calculated by one-way ANOVA.

FIG. 21. PARPi/PI3Ki-mediated tumor regression in B6-Myc (STING^{hi}) model occurs via a DC-independent mechanism. B6-Myc (STING^{hi}) tumor-bearing syngeneic mice were treated with indicated drugs(s) until untreated tumors reached approx. 2500 mm³. Single-cell suspensions were generated from harvested tumors and analyzed by flow cytometry for % activated DCs gated on CD45+CD11b+MHC-II+CD11C+. Data are represented relative to CD45+ immune cells. n=3-4 per group.

FIGS. 22A-22D. Concomitant clodronate treatment decreases MHC-I expression on CD45− cells in B6-Myc (STING^{hi}) tumor-bearing mice that received PARPi/PI3Ki treatment; PARPi/PI3Ki combination therapy causes tumor regression in B6-Myc (STING^{hi}) murine PC via host STING-dependent immune mechanism. (22A) B6-Myc (STING^{hi}) tumor-bearing syngeneic mice were treated with PARPi/PI3Ki+/− clodronate (to deplete macrophages) until untreated tumors reached 2500 mm³. Tumors were processed and stained for MHC-I by flow cytometry. (22B) C57Bl/6J^{STING+/+} and C57Bl/6J^{STING-/-} mice were engrafted with B6-Myc (STING^{hi}) tumor allografts and treated with the indicated drug(s) until untreated tumors reached approx. 2500 mm³. Tumor volume curves for duration of treatment are shown. Single-cell suspensions were generated from harvested tumors and analyzed by flow cytometry for activated macrophages (22C) and activated T cells (22D). Data are represented relative to CD45+ immune cells. n=3 mice per group; Significance/p-values were calculated by one-way ANOVA.

FIG. 23. Myc-CAP (STING^{lo}) tumors do not respond to PARPi/PI3Ki treatment in the absence of castration. Myc-CAP (STING^{lo}) tumor-bearing uncastrated syngeneic mice were treated with the indicated drug(s) until untreated tumors reached approx. 2500 mm³. Tumor volumes were recorded for duration of treatment; n=4 animals per group. Significance/p-values were calculated by one-way ANOVA.

FIGS. 24A-24G. Chemical castration increases macrophage and CD4+ T cell infiltration and PD-L1 expression within the TME of Myc-CAP (STING^{lo}) tumors. Myc-CAP (STING^{lo}) tumor-bearing syngeneic mice were treated with degarelix (chemical castration) for 10 days. At the end of treatment, tumors were harvested and analyzed by flow cytometry for infiltration of CD45+ immune cells (24A), macrophages (24B) and CD4/CD8 T cells (24C-24D). Mean fluorescence intensity (MFI) is depicted for PD-Ll expression on CD45−/CD45+ cells (24E-24F) and TAMs (24G) within the TME; n=3-5 animals/group; Significance/p-values were calculated by Un-paired t-test.

FIGS. 25A and 25B. ADT/PARPi/PI3Ki combination induces TAM-driven tumor control in Myc-CAP (STING^{lo}) syngeneic mice. (25A-25B) Myc-CAP (STING^{lo}) tumor-bearing syngeneic mice were treated with the indicated drug(s) until untreated tumors reached approx. 2500 mm³. Tumor volume curves for duration of treatment are shown.

FIGS. 26A-26D. The anti-tumor response elicited by ADT/PARPi/PI3Ki is abrogated in Myc-CAP (STING^{lo})-tumor bearing athymic nude and NOD/SCID mice, ADT/PARPi/PI3Ki combination induces TAM-driven tumor control in Myc-CAP) (STING^{lo}) syngeneic mice. Immunodeficient athymic nude (26A) and NOD-SCID (26B) mice were engrafted with Myc-CAP (STING^{lo}) tumors and treated with the indicated drug(s) until untreated tumors reached approx. 2000 mm³. Tumor volume curves for duration of treatment are shown. For 26A-26B, n=3-4 mice per group. (26C-26D) Single-cell suspensions were generated from harvested tumors and analyzed by flow cytometry for the indicated immune cell populations and their activation states. Data are represented relative to CD45+ immune cells. For 26C-26D, n=3-5 animals per treatment group from 2 independent experiments. Significance/p-values were calculated by one-way.

FIGS. 27A-27D. ADT/PARPi/PI3Ki with/without PD-L1 antibody treatment increases infiltration and activation of CD4 T cells within the TME of STING^{lo} Myc-CAP tumors, ADT/PARPi/PI3Ki combination induces TAM-driven tumor control in Myc-CAP) (STING^{lo}) syngeneic mice. Myc-CAP (STING^{lo}) tumor-bearing syngeneic mice were treated with the indicated drugs until untreated tumors reached approx. 2500 mm³. Single-cell suspensions were generated from harvested tumors and analyzed by flow cytometry for CD4 infiltration (27A) and activation (27C). (27B, 27D) Single-cell suspensions were generated from harvested tumors and analyzed by flow cytometry for the indicated immune cell populations and their activation states. Data are represented relative to CD45+ immune cells. n=3-5 animals per treatment group from 2 independent experiments. Significance/p-values were calculated by one-way ANOVA.

FIGS. 28A and 28B. ADT/PARPi/PI3Ki treatment enhances M1 macrophage polarization within Myc-CAP (STING^{lo}) tumors in vivo. (28A-28B) Myc-CAP (STING^{lo}) tumor-bearing syngeneic mice were treated with the indicated drugs until untreated tumors reached approx. 2500 mm³. Tumors were harvested for qRT-PCR analysis to interrogate changes in il12b expression (28A). (28B) Single-cell suspensions were generated from harvested tumors and analyzed by flow cytometry for macrophage (F4/80+) Arginase I expression. n=3 animals/group; Significance/p-values were calculated by one-way ANOVA.

FIGS. 29A-29M. PARPi alone induces DNA DSBs, but not apoptosis even in combination with PI3Ki in Myc-CAP (STING^{lo}) cancer cells; PARPi/PI3Ki combination induces intracellular DNA DSBs and proportionate release of MV surface-associated DNA DSBs from B6-Myc (STINGhi) cancer cells without affecting cellular viability; PARPi/PI3K treatment activates tumor-cell extrinsic cGAS/STING pathway within TAMs in vivo via MV surface-associated DNA DSBs released from tumor cells. Myc-CAP cancer cells were treated with the indicated drugs singly or in combination for 36 hours. (29A) Cells were stained with anti-mouse specific p-γH2AX antibody and a fluorescently labeled secondary antibody for determination of DNA DSBs, which were quantified by confocal microscopy. (29B) Protein extracts from cells in 29A were analyzed for the indicated pharmacodynamic biomarkers by western blotting; (29C) Annexin V-PI staining was done to assess frequency of live cells (Annexin V⁻ PI⁻) following drug treatment. (29D) Ultracentrifugation was utilized to purify microvesicles (MVs) from supernatants in treatment groups in (29A) and associated DNA DSBs was quantified by Nanodrop®. Independent experiments n=2. (29E) Ultracentrifugation was utilized to purify MVs from supernatants in indicated treatment groups in FIG. 17A, and associated DNA DSBs were quantified by Nanodrop®; n=2 independent experiments. (29F) Syngeneic mice were engrafted with Myc-CAP (STING^{lo}) cells, and tumors were harvested when the tumor volume reached 400 mm³. Single cell suspensions of the tumors were treated with the indicated drug(s), in the presence or absence of GW4869 (MV biogenesis inhibitor). (29G) Bone marrow derived macrophage (BMDMs) were co-cultured for 30 hours with supernatants (−/+ exogenous 50 units of DNase I pre-treatment for 30 minutes) from Myc-CAP cells that were treated with the indicated drug(s) for 36 hours. (29H) cGAMP (10 μg/mL) was pre-incubated with 10 units of DNAse I or mock control for 30 minutes and then added to BMDMs for 30 hours. (29I-29L) STING^{+/+}/STING^{-/-} BMDMs were co-cultured for 36 hours with supernatants from Myc-CAP and B6-Myc cancer cells that were treated with the indicated drug(s) for 36 hours. Supernatants were collected from 29F-29L at the end of treatment and analyzed for IFNβ1 by ELISA. (29M) Myc-CAP (STING^{lo}) tumor-bearing mice were treated with the indicated drug(s) until tumors first reached approx. 2500 mm³. For the degerelix/rucaparib/buparlisib combination, additional cohorts of mice underwent concomitant treatment with STING antagonist H-151 or GW4869. Tumor volume was recorded daily for duration of experiment. For in vitro experiments, n=2 independent experiments and for in vivo experiments n=3-4 animals /group. Significance/p-values were calculated by one-way ANOVA.

FIGS. 30A-30G. Buparlisib inhibits activation of the PI3K/AKT pathway within BMDMs, optimal STING activation within macrophages requires both PARPi-mediated MV surface-associated DNA DSBs and PI3Ki-induced de-repression of cGAS enzymatic activity. (30A) BMDMs were co-cultured with MVs isolated from Myc-CAP) (STING^{lo}) cancer cells following 36 hours treatment with rucaparib (R-MVs), in the presence or absence of buparlisib at the indicated concentrations for 24 hours. Protein extracts were harvested for assessment of PI3K target inhibition by western blotting; U=Untreated. (30B) Experimental schema: Myc-CAP (STING^{lo}) cells were treated with rucaparib (0.5 μM) for 36 hours, followed by isolation of MVs from supernatant (R-MVs), which were then co-cultured with BMDMs in the presence/absence of buparlisib (1 μM) for 36 hours. BMDMs were directly treated with DMXAA (50 μg/mL). (30C-30D) Cellular metabolites and proteins extracted from co-cultured BMDMs (as per schema in 30B) were used for cGAMP measurement via ELISA (30C) and for assessment of activation of STING pathway by western blotting using indicated antibodies (30D), respectively. (30E-30F) Supernatants collected from experiment in (30B) were used for IFN-α (30E) and IFN-β (30F) measurement via ELISA. (30G) Model for cGAS/STING pathway activation within suppressive macrophages (M2) following treatment with buparlisib and MVs (isolated from rucaparib-treated Myc-CAP cells); n=2 independent experiments. Significance/p-values were calculated by one-way.

FIGS. 31A-31G. The combination of buparlisib with MV surface-associated DNA DSBs from PARPi treated cancer cells reprograms macrophages from an M2 to M1 phenotype; Conditioned medium from PARPi/PI3Ki treated Myc-CAP (STING^{lo}) cells reprograms BMDMs from an M2 to M1 phenotype. Myc-CAP (STING^{lo}) cells were treated with rucaparib (0.5 μM) for 36 hours, followed by isolation of MVs from supernatant (R-MVs), which were then co-incubated with BMDMs in the presence/absence of buparlisib (1 μM) for 36 hours. As a positive control, BMDMs were directly treated with DMXAA (50 μg/mL). At the end of treatment, BMDMs were analyzed by flow cytometry for macrophage activation markers to quantify frequency of CD45+CD11b+F4/80+ cells expressing MHC-II (31A) and CD86 (31B). (31C-31E) Supernatants were collected for determination of Ml-specific cytokines/chemokines by cytokine array; n=2 independent experiments. (31F-31G) BMDMs were co-cultured for 36 hours with supernatants from Myc-CAP (STING^{lo}) cancer cells that were treated with the indicated drug(s) (rucaparib at 0.5 μM, buparlisib at 1 μM) for 36 hours. DMXAA (50 μg/mL) was directly added to BMDMs. Following treatment, BMDMs were stained for expression of markers (31F) CSF-IR; (31G) PD-L1; n=2 independent experiments; Significance/p-values were determined by one-way ANOVA (A)/Un-paired t-test.

FIGS. 32A-32F. Conditioned medium from PARPi/PI3Ki treated Myc-CAP) (STING^{lo}) cells reprograms BMDMs from an M2 to M1 phenotype. BMDMs were co-cultured for 36 hours with supernatants from Myc-CAP (STING^{lo}) cancer cells that were treated with the indicated drug(s) (rucaparib at 0.5 buparlisib at 1 μM) for 36 hours. DMXAA (50 μg/mL) was directly added to BMDMs. Following treatment, BMDMs were stained for expression of macrophage activation markers (32A) MHC-II; (32B) iNOS and suppressive marker (32C) Arginase I on CD45+ CD11b+F4/80+ macrophages. (32D-32E) Supernatants were collected for determination of secreted chemokines by cytokine array; n=2 independent experiments; Significance/p-values were determined by one-way ANOVA. (32F) Working Model: The combination of PARPi+PI3Ki induces intracellular DNA DSBs that become associated with the surface of extracellular MVs following release into the TME. In addition, PI3Ki can inhibit AKT-mediated Ser-291 phosphorylation of cGAS, thus de-repressing its enzymatic activity. MV surface-associated DNA DSBs can secondarily activate the cGAS/STING pathway within TAMs only in the presence of concomitant PI3Ki, resulting in macrophage activation and M2 to M1 polarization, increased T cell infiltration, and tumor regression in c-myc driven PC.

FIGS. 33A and 33B. Prostate cancer patients with poor-prognosis are enriched for PTEN loss-of-function. Integrative analysis was performed on 488 primary prostate cancer TCGA samples with RNA sequencing, promoter site methylation, somatic mutation, and somatic copy number variation. The clustering was performed with iClusterPlus using 5380 genes that were differentially expressed between previously published “low” and “high” Prostate Cancer Metastatic (PCM) signature, and interrogated for PTEN homozygous (33A) or heterozygous (33B) copy number.

FIGS. 34A-34D. Human and murine PTEN-deficient PCs have enhanced MDSC and diminished T cell infiltration within their TME, relative to their PTEN-proficient counterparts. (34A) Primary prostate tumors (n=158, TCGA) and metastatic prostate tumors (n=43, Stand Up to Cancer) were delineated based on genomic loss of PTEN (at least one copy), and then stratified based on an MDSC-high or MDSC-low gene-expression signature of 14 genes. Pooled bioinformatics analysis for PTEN-deficient primary (n=158) and PTEN-deficient metastatic prostate cancers (n=43) were obtained from the TCGA and SU2C databases, respectively. (34B) PTEN-deficient tumors of all histologies from the TCGA database (n=6153 from 30 different tumor histologies) were stratified based on the presence or absence of a published T cell-inflamed or non-T cell-inflamed gene signature. (34C) Percentage distribution of different immune cell subsets in PTEN-proficient vs. PTEN-deficient murine syngeneic Myc-CAP tumors. Subcutaneous tumors were harvested at a tumor volume of 2000 mm³ and analyzed by flow cytometry. (34D) PTEN-proficient vs. PTEN-deficient Myc-CAP cells were cultured for 24 hours, and cytokine array analyses were performed on supernatants.

FIGS. 35A-35D. Copanlisib retards growth of Myc-CAP-PTEN^{-/-} tumors by decreasing CXCL1 production and MDSC infiltration. (35A) Myc-CAP-PTEN^{-/-} cells were treated with copanlisib 100 nm (IC90 concentration) in vitro for 24 hours, and protein extracts were harvested for western blotting analysis with the indicated antibodies to analyze PI3K activation. (35B) Supernatants from (35A) were harvested for cytokine array profiling. n=3 independent in vitro experiments. (35C) Mice bearing Myc-CAP-PTEN^{-/-} established syngeneic tumors (200 mm³) were treated with copanlisib (14 mg/kg, every other day, until tumors reached 2000 mm³). Tumor volumes were monitored daily and graphed. (35D) At the end of treatment, tumors were analyzed by flow cytometry for tumor infiltrating immune cell subsets. n=5 mice per group.

FIG. 36. ADT-induced CD45+ infiltration within Myc-CAP-PTEN-knockout tumors is reversed following the development of castration resistance. Myc-CAP-PTEN knockout syngeneic tumor cells were engrafted subcutaneously within FVB mice. When tumors reached 200 mm³, mice were treated with degarelix (chemical castration) for 10 days, and tumors were harvested for both castrate-sensitive and castrate-resistant mice, and analyzed for CD45+ cells by flow cytometry.

FIGS. 37A and 37B. Myc-CAP-PTEN deficient cancers are resistant to ADT/PARPi/PI3Ki/combination due to an absence of macrophage infiltration/activation and secondary CD8/CD4 T cell infiltration following treatment. Syngeneic PTEN-proficient and PTEN-deficient Myc-CAP mice were treated with a single dose of degarelix, rucaparib (PARP inhibitor), BKM120 (pan-PI3K inhibitor), or anti-PD-L1 antibody, singly and in combination for 3 weeks. (37A) Tumor volumes were monitored, as indicated. (37B) Tumors were harvested at the end of treatment and macrophage infiltration and activation (upper left and right panels), and CD4 and CD8 T cell infiltration (lower left and right panels, respectively) were analyzed by flow cytometry. n=3 animals/group.

FIG. 38. PTEN-deficient prostate cancers are resistant to DNA damage-induced c-GAS/STING pathway activation within the TME. Single cell suspensions from Myc-CAP-PTEN+/+ and Myc-CAP-PTEN−/− syngeneic tumors were treated with indicated drugs or combinations at the following IC90 concentrations: Rucaparib at 500 nM, Buparlisib at 1000 nM, and DMXAA at 50 μg/mL for 36 hours. Supernatants collected at end of treatment were used for detection of IFNB1 by ELISA; n=2 independent experiments.

FIG. 39. DMXAA treatment induces STING activation in prostate-specific PTEN^{-/-}p53^{-/-} GEMM tumors. Tumors from prostate-specific PTEN^{-/-}p53^{-/-} GEMM tumors were processed into single cell suspensions and treated with indicated drugs (DMXAA at 50 μg/mL, Buparlisib at 1000 nM, Rucaparib at 500 nM). At 36 hours supernatants were collected for ELISA based detection of IFNβ1; n=1 independent experiment.

FIGS. 40A and 40B. Exosomes released from PARPi-treated Myc-CAP-PTEN-deficient cancer cells have significantly reduced dsDNA content, relative to their PTEN-proficient counterparts. Isogenic PTEN-proficient and CRISPR-CAS9-mediated PTEN-knockout Myc-CAP cells were treated with rucaparib for 36 hours. Exosomes were collected by ultracentrifugation and used for quantification of particle number by Nanosight (40A) and dsDNA content by Nanodrop® (40B). n=2 experiments.

FIGS. 41A and 41B. Exosomes released from PARPi-treated Myc-CAP-PTEN-deficient cancer cells have significantly reduced dsDNA content, relative to their PTEN-proficient counterparts. siRNA mediated PTEN-knockdown Myc-CAP cells corresponding to those used in FIGS. 40A and 40B were treated with rucaparib for 36 hours. Exosomes were collected by ultracentrifugation and used for quantification of particle number by Nanosight (41A) and dsDNA content by Nanodrop® (41B). n=2 experiments.

FIGS. 42A and 42B. Optimal STING activation within macrophages requires both PARPi-mediated exosome/associated DNA and PI3Ki-induced de-repression of cGAS enzymatic activity. (42A) Experimental schema: Myc-CAP^{pten-/-} cells were treated with PARPi for 36 hours followed by isolation of exosomes from supernatant. Exosomes were then co-incubated with BMDMs in the presence/absence of buparlisib for 36 hours. (42B) Supernatants collected from experiment in (42A) were used for measuring IFNβ1 levels via ELISA; n=2 independent experiments. Metabolites and proteins extracted from co-cultured BMDMs from (42A) were used for cGAMP ELISA (42C) and STING pathway signaling analysis by Western Blotting (42D), respectively; n=2 independent experiments. (42E) Supernatants collected from experiment in (42A) were used for measuring IFNβ1 levels via ELISA; n=1 independent experiment. (42F) Model for cGAS/STING pathway activation within macrophages following treatment with exosomes (isolated from rucaparib-treated Myc-CAP cells) and buparilisib.

FIGS. 43A and 43B. Increased levels of negatively charged phosphatidylserine (PS) on the surface of both cellular and exosomal membranes derived from Myc-CAP-PTEN^{-/-} cells relative to PTEN-proficient counterparts. Isogenic Myc-CAP cells (−/+PTEN) and their corresponding exosomes were stained with Annexin V (which binds to surface PS) for 30 minutes and analyzed by flow cytometry. Shown here is the mean fluorescence intensity of stain for (43A) cell lines (43B) exosomes; n=1 independent experiment.

FIG. 44. PTEN-deficient Myc-CAP tumors are paradoxically more responsive to STING agonist treatment in vivo, relative to their PTEN-proficient counterparts. Syngeneic mice bearing established PTEN-proficient (PTEN^{+/+}) and PTEN-deficient (PTEN^{-/-}) Myc-CAP tumors (200 mm³) were treated with DMXAA (500 single intra-tumoral dose) for 12 days. Tumor volumes were monitored daily and graphed relative to untreated groups. n=3-5 mice per group.

FIGS. 45A and 45B. STING agonist increases global immune infiltration and has a trend towards decreasing Gr-MDSCs within Myc-PTEN-knockout tumors relative to their PTEN-proficient counterparts. Syngeneic mice bearing established PTEN-proficient (PTEN^{+/+}) and PTEN-deficient (PTEN^{-/-}) Myc-CAP tumors (200 mm³) were treated with DMXAA (500 μg, single intra-tumoral dose) for 12 days. The harvested tumors were analyzed by flow cytometry for CD45 (45A) and tumor infiltrating immune cell subsets (45B). n=3-5 mice per group.

FIGS. 46A-46C. STING agonist treatment selectively increases macrophage, CD4 and CD8 T-cell activation within Myc-PTEN-knockout tumors, relative to their PTEN-proficient counterparts. Syngeneic mice bearing established PTEN-proficient (PTEN^{+/+}) or PTEN-deficient (PTEN^{-/-}) Myc-CAP tumors (200 mm³) were treated with DMXAA (500 μg, single intra-tumoral dose) for 12 days. The harvested tumors were analyzed by flow cytometry for activation status of immune cells: (46A) MHC-II/PD-L1 expression on macrophages; (46B) 41bb/PD-1 expression on CD4+FoxP3-T-cells and (46C) on CD8+T-cells. n=3-5 mice per group.

FIG. 47. STING agonist treatment increases regulatory T-cell infiltration in PTEN deficient Myc-CAP tumors relative to PTEN-proficient counterparts. Syngeneic mice bearing established PTEN-proficient (PTEN^{+/+}) and PTEN-deficient (PTEN^{-/-}) Myc-CAP tumors (200 mm³) were treated with DMXAA (500 μg, single intra-tumoral dose) for 12 days. The harvested tumors were analyzed by flow cytometry for CD4+T-cells subsets, specifically FoxP3+ and FoxP3− fractions. n=3-5 mice per group.

FIGS. 48A and 48B. Direct STING agonist treatment increases CD8 T cell infiltration and relieves myeloid immunosuppression in prostate GEMM tumors that develop in the context of PTEN and p53 loss. PTEN/p53-deficient GEMM mice with established tumors were treated with DMXAA every other day for 14 days. Tumors were harvested and subjected to flow cytometry for detection of the indicated markers. (48A) CD8 infiltration; (48B) M1/M2 ratio of F4/80⁺ cells where M1 are defined as MHC-II⁺ and M2 cells are MHC-II⁻. The frequency of Gr-MDSCs (Ly6C⁻Ly6G⁺) and surface expression of V-domain Ig suppressor of T cell activation (VISTA) and PD-L1 on Gr-MDSCs are shown.

FIGS. 49A and 49B. PTEN-deficient cancer cells have diminished STING expression that can be restored by PI3KI3-selective and pan-PI3K inhibitors. (49A) Isogenic MC38, Myc-CAP, ID8, and TRAMP cells (−/+ PTEN) were analyzed for STING mRNA and protein expression by qRT-PCR and western blotting, respectively. (49B) Myc-CAP-PTEN^{-/-} cells were treated with GSK′771 (PI3Kβ-selective inhibitor, 1 μM) or BKM120 (pan-PI3K inhibitor, 1 μM) for 24 hours, and proteins were analyzed for STING expression and PI3K activation by western blotting.

FIG. 50. STING agonist in combination with PI3K inhibitors activated type I interferon production within the TME of Myc-CAP-PTEN^{-/-} prostate tumors. Single cell suspensions from syngeneic Myc-CAP-PTEN^{-/-} tumors were treated ex vivo with the indicated drugs for 36 hours. Supernatants were harvested for cytokine array profiling and quantitative assessment of IFNB1 and CXCL10. Drugs were used at following IC90 concentrations: DMXAA (50 μg/mL), Buparlisib (1000 μuM), Copanlisib (500 nM), CAL101 (500 nM), IPI549 (500 nM), and GSK771 (500 nM).

FIGS. 51A-51C. Forced overexpression of STING within myc-CAP cells delays tumor growth in vivo relative to wild-type counterparts. (51A) Myc-CAP (STING^{lo}) cells were transfected with plasmid to overexpress STING (STING OE). Western blot analysis was performed to confirm increase in expression of STING relative to parent cell line and macrophages. n=1 independent experiment. (51B) Syngeneic mice bearing tumors from established wild-type and indicated engineered variants of Myc-CAP were monitored for tumor growth and graphed until they reached the cut-off tumor volume (2500 mm³). (51C) Kaplan-Meier curves for Myc-CAP wild-type and indicated engineered variants.

FIG. 52. STING agonist treatment is protective to mice bearing Myc-CAP tumors with Intermediate (not high) tumor cell intrinsic levels of STING. Syngeneic mice bearing Myc-CAP wild-type (WT) and STING overexpressed (STING OE; clone 34) or STING intermediate (STING OE; clone 35) tumors (200 mm³) were treated with DMXAA (500 single intra-tumoral dose). Tumors were monitored until cut-off tumor volume of 2500 mm³, and Kaplan Meier curves were plotted. n=3 mice per cohort.

FIGS. 53A-53C. STING overexpression drives STING signaling pathway activation and type 1 IFN secretion from Myc-CAP prostate cancer cells in direct proportion to the level of tumor cell intrinsic STING expression. Wild-type and STING overexpressing (STING OE, clone 34) Myc-CAP cells were treated with mouse STING agonist, DMXAA (50 μg/mL) for 1 and 6 hours to perform western blot to assess STING signaling pathway activation (53A) and qRT-PCR for IFNB1 expression analysis (53B), respectively. (53C) Wild-type (WT), PTEN-deficient (PTEN^{-/-}), Rb-deficient (Rb^{-/-}) and STING overexpressed (SOE34 (clone 34) or SOE35 (clone 35)) Myc-CAP cells were treated with DMXAA (50 μg/mL, 36 hours). The supernatants were collected from each well and IFNB1 analyzed by ELISA. n=2 independent experiments.

FIG. 54. PTEN-deficient syngeneic ID8 murine ovarian cancer model has higher lethality relative to other isogenic models, all of which recapitulate clinical features of high-grade serous ovarian cancer.

FIG. 55. PTEN loss suppresses tumor cell intrinsic STING expression within ID8 cancer cells relative to wild type controls, which is partially reversed with concomitant p53 loss. Protein extracts from the indicated ID8 cancer cells were probed for AKT and STING pathway components by western blotting. n=1 independent experiment.

FIGS. 56A and 56B. PTEN knockdown in ID8 ovarian cancer cells abrogates STING pathway activation following DMXAA treatment. (56A) PTEN-proficient (PTEN^{+/+}) and deficient (PTEN^{-/-}) ID8 ovarian cancer cells were treated with DMXAA (50 μg/mL) for 0, 1, 6, 12, 24 and 36 hours. Nuclear extracts were probed for localization of p-IRF3 and p-NF-kB, markers of STING pathway activation. n=1 independent experiment. (56B) Wild-type (PTEN^{+/+}) and PTEN-deficient (PTEN^{-/-}) Myc-CAP (prostate) and ID8 (ovarian) cancer cells were treated with DMXAA (50 μg/mL) for 36 hours. The supernatants were analyzed by ELISA for IFN-β1 release. n=3 independent experiments.

FIGS. 57A-57C. PTEN knockdown in ID8 ovarian cancer cells abolishes IRF3 translocation to the nucleus following DMXAA treatment. PTEN-proficient (PTEN+/+) and deficient (PTEN−/−) ID8 ovarian cancer cells were treated with DMXAA (50 μg/mL) for 0, 12, 24 and 36 hours. (57A) The nuclear localization of p-IRF3 and p-NFkB was analyzed by ImageStream. The overlap of the two generates a white color that scores for nuclear localization of pIRF3. (57B) Graphical representation of nuclear localization of pIRF3 and p-NF-kB at 12, 24, and 36 hours after DMXAA treatment, relative to untreated groups in ID8 wild-type and PTEN^{-/-} cells. n=3 independent experiments. (57C) Schematic model demonstrating defect in nuclear localization of IRF3 and loss of IFNB1 secretion from PTEN deficient ID8 cells.

FIGS. 58A and 58B. PI3K inhibitors partially restore STING pathway activation within ID8-PTEN^{-/-} cells but are unable to rescue IFNB1 levels in combination with STING agonists. (58A) PTEN-proficient (PTEN^{+/+}) and deficient (PTEN^{-/-}) ID8 ovarian cancer cells were treated with IC90 concentrations of pan-PI3K inhibitors, either copanlisib (100 nM) or BKM120 (1000 nM) for 36 hours in presence or absence of STING agonists, DMXAA (50 μg/mL, 1 hour at the end of experiment). The activation status of both PI3K and STING signaling pathways were probed with the indicated antibodies by western blotting. n=2 independent experiments. (58B) Cells were treated as described in (58A), with the exception that concomitant DMXAA treatment was carried out for 36 hours. The supernatants were analyzed by ELISA for IFN-β1 release. n=3 independent experiments.

FIG. 59. Mechanistic Model for PTEN loss-mediated downregulation of STING signaling. PTEN loss suppresses STING signaling via multiple mechanisms, which includes suppression of STING expression and sequestration of p-IRF3 within the cytosol, the latter being due to persistent Ser-97 phosphorylation.

FIGS. 60A-60C. STING agonist/PI3Ki combination therapy provides durable tumor control and dramatically increases survival of PTEN-deficient advanced ovarian cancers. C57Bl/6J mice received an intraperitoneal injection of 5 million syngeneic ID8-PTEN^{-/-} cancer cells. Treatment with the indicated drug(s) was initiated when mice developed ascites and abdominal girth was at 23-24 mm, and terminated when the abdominal girth reached 35 mm or animals displayed visible signs of distress due to disease progression or visible ascitic fluid regression at approximately 80-90 days. (60A) Abdominal Girth; (60B) Weight; (60C) Percent survival; drugs were used at following doses: DMXAA (500 μg, i.p. once), Copanlisib (14 mg/kg, i.v. daily), Buparlisib (30 mg/kg, oral gavage, daily), Rucaparib (150 mg/kg, oral gavage, daily). n=2-3 mice/group.

FIGS. 61A-61C. STING agonist/PI3Ki combination therapy increases survival of PTEN/p53-deficient advanced ovarian cancers via tumor cell extrinsic STING pathway activation. (61A) C57Bl/6J mice received an intraperitoneal injection of 5 million syngeneic ID8-PTEN^{-/-}p53^{-/-} cancer cells. Treatment with the indicated drug(s) was initiated when mice developed ascites and abdominal girth was at 23-24 mm, and terminated when the abdominal girth reached 35 mm or animals displayed visible signs of distress due to disease progression. Percent survival following treatment is shown; drugs used at following concentrations: DMXAA (500 μg, i.p. once), Buparlisib (30 mg/kg, oral gavage, daily). n=2-3 animals/group. Cell lines (61B) and single cell suspensions isolated from ascitic fluid (61C), were treated with the indicated drug(s) at IC90 concentrations for 36 hours. Supernatants were collected for IFNB1 ELISA. n=1 independent experiment

FIGS. 62A and 62B. STING overexpression rescues Type 1 IFN production within ID8-PTEN^{-/-} ovarian cancer cells. (62A) ID8-PTEN^{-/-} cells were stably transfected with STING expression plasmid, and positive clones were selected based on GFP-based cell sorting and puromycin selection. Western blot analysis was performed to confirm increase in expression of STING relative to parent cell line and macrophages. n=1 independent experiment. (62B) ID8-Wild-type (WT), ID8-PTEN-deficient (KO) and ID8-PTEN-deficient/STING overexpressed (KO STING OE, clone 23 (SOE23)) ovarian cancer cells were treated with DMXAA (50 μg/mL) for 36 hours. The supernatants were collected from each well and IF191 analyzed by ELISA. n=3 independent experiments. Untr=untreated; My=macrophages.

FIG. 63. The immune infiltrates in human mCRPC and murine Myc-driven PC models are dominated by myeloid suppressive cells, particularly tumor-associated macrophages (TAMs). Single cell suspensions of human mCRPC biopsies, murine Myc-CAP) (STING^{lo}) and B6-myc (STING^{hi}) syngeneic tumors were stained for the following markers: CD45+ (total immune), CD3+ (lymphoid), CD11b+, F4/80+ (macrophage), or CD11b+, F4/80− (“other” myeloid), and analyzed by flow cytometry. n=2 for human and n=3-4 for murine samples.

FIGS. 64A and 64B. PC patients with poor prognosis exhibit reduced STING expression and myeloid activation markers. (64A) c-GAS and STING expression in normal vs. PC within the TCGA. (64B) Bioinformatic clustering was performed with iClusterPlus using 5380 genes on 488 primary PC TCGA samples that were differentially expressed between previously published “low” and “high” Prostate Cancer Metastatic (PCM) signatures. Four patient subgroups were identified using such analysis: C1, C2, C3, and C4. The C4 cluster was reported to have a higher proportion of DDR genes and poor prognosis relative to other clusters. Expression of STING, and indicated myeloid activation markers between C4 (n=64) vs other clusters C1+C2+C3 (n=362). CIITA, Class II Major Histocompatibility Complex Transactivator.

FIGS. 65A and 65B. PARPi/PI3Ki Induces Tumor Regression in syngeneic B6-myc (STING^{hi}), but not Myc-CAP (STING^{lo}) tumors. B6-myc (65A) and Myc-CAP (65B) syngeneic tumor-bearing mice were treated with DMXAA (mouse STING agonist), rucaparib (PARP inhibitor), and buparlisib (pan-PI3K inhibitor) or indicated combination for 2 weeks, tumor growth was measured daily. n=2-3 mice per arm.

FIGS. 66A-66D. PARPi/PI3K combination treatment induces macrophage-driven tumor control in castrated Myc-CAP (STING^{lo}) syngeneic mice. Myc-CAP (STING^{lo}) tumor-bearing syngeneic mice were: (66A) treated with single dose of degarelix for 10 days and tumors were harvested and analyzed for the indicated markers by flow cytometry. (66B) treated with degarelix, rucaparib (PARPi), buparlisib (PI3Ki), singly and in combination for 21 days, as indicated, and tumor volumes were measured as indicated. (66C) treated as in (66B), with the addition of cohorts that received concomitant anti-PD-L1 antibody. (66D) treated as in (66B), with the addition of a cohorts that received concomitant clodronate, which depletes macrophages. n=2-4 animals per group.

FIGS. 67A-67C. DNA damage-induced STING pathway activation within tumor-associated macrophages is mediated via dsDNA-containing microvesicles. (67A-67B) Single cell suspensions from 400 mm³ Myc-CAP (STING^{lo}) tumors were treated ex vivo with indicated drugs for 36 hours−/+GW4869, exosome biogenesis inhibitor (67A) or DNase I (67B), and supernatants were collected and assayed for IFNB1; (67C) STING+/+/STING−/− BMDMs were co-cultured for 36 hours with supernatants derived from tumor cells that were treated with DMXAA, rucaparib, buparlisib, or a combination of rucaparib/buparlisib for 36 hours. Co-culture supernatants were collected and analyzed by ELISA for IFNB1; n=2 experiments. Asterisks without line (*) indicate comparison of target group with untreated.

FIG. 68. In vivo inhibition of exosome biogenesis and STING activation following ADT/PARPi/PI3Ki treatment reverses tumor regression. FVB mice bearing syngeneic Myc-CAP (STING^{lo}) tumors were treated with the indicated drugs (D=Degarelix, R=Rucaparib; and B=Buparlisib) for 2-4 weeks in the presence or absence of exosome inhibitor GW4869 or STING antagonist H-151. n=2-3 animals per group.

FIGS. 69A-69D. Optimal STING activation within TAMs requires both PARPi-induced DNA DSBs associated with MVs and PI3Ki-induced de-repression of c-GAS enzymatic activity. (69A) Schema showing Myc-CAP (STING^{lo}) cells treated with PARPi for 36 hours followed by isolation of exosomes from supernatant, which were then co-cultured with BMDMs in the presence or absence of buparlisib for 36 hours. (69B) Supernatants were collected for IFNB1 ELISA. Metabolites (69C) and proteins (69D) were extracted from co-cultured BMDMs and analyzed for cGAMP levels and STING signaling by ELISA and western blotting, respectively. n=2 experiments.

FIG. 70. Therapeutic strategies to overcome myeloid immunosuppression and enhance T cell infiltration and ICB-responsiveness in PTEN-deficient prostate cancer.

FIGS. 71A and 71B. Copanlisib treatment increases CD4 and CD8 T-cell infiltration and decreases Gr-MDSC and Tregs within the TME of PTEN/p53-deficient prostate GEMMs. PTEN/p53-deficient GEMM mice (n=2/group) with established tumors were treated with copanlisib (14 mg/kg/day, every alternate day) for 4 weeks. Tumors were collected and analyzed for changes in global immune infiltrates, shown clockwise from twelve o'clock: CD4+, CD8+, CD19+, F4/80+, CD11c+, Mo-MDSC, Gr-MDSC, neutrophils (71A) and Treg frequency (71B) by flow cytometry. n=2 mice per arm.

FIG. 72. Phase Ib/II investigator initiated trial of BMS-986301 (systemic STING agonist) in combination with copanlisib (PI3K inhibitor) and nivolumab (PD-1 antibody) in mCRPC patients.

### DETAILED DESCRIPTION OF PREFERRED EMBODIMENTS

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, patents and patent applications cited herein are hereby expressly incorporated by reference in their entirety for all purposes.

### Definitions

Before describing the methods and compositions of the disclosure in detail, a number of terms will be defined.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other component, feature, element, or step or group of component, feature, element, or steps.

As used in the specification and the appended claims, the singular forms “a,” “an,” and “the” include plural referents unless the context clearly dictates otherwise.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

It is noted that terms like “preferably,” “commonly,” and “typically” are not utilized herein to limit the scope of the methods and compositions as described herein or to imply that certain features are critical, essential, or even important to the structure or function of the claimed invention.

As used herein, ranges and amounts can be expressed as “about” a particular value or range. About also includes the exact amount. Hence “about 5%” means “about 5%” and also “5%.” The term “about” can also refer to ±10% of a given value or range of values. Hence, about 5% also means 4.5%-5.5%, for example.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, the terms “therapeutic amount,” “therapeutically effective amount,” or “effective amount” can be used interchangeably and refer to an amount of a compound, such as a therapeutic agent, that becomes available through an appropriate route of administration to treat a patient for a disorder, a condition, or a disease. The amount of a compound which constitutes a “therapeutic amount,” “therapeutically effective amount,” or “effective amount” will vary depending on the compound, the disorder and its severity, and the age of the subject to be treated, but can be determined routinely by one of ordinary skill in the art.

“Treating” or “treatment” as used herein covers the treatment of a disease or disorder described herein, in a subject, preferably a human, and includes:
i. inhibiting a disease or disorder, i.e., arresting its progression;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder.
   For example, the terms “treating,” “treat,” or “treatment” refer to either preventing, providing symptomatic relief, or curing a patient's disorder, condition, or disease.

As used herein, the terms “patient” and/or “subject” and/or “individual” can be used interchangeably and refer to an animal. For example, the patient, subject, or individual can be a mammal, such as a human to be treated for a disorder, condition, or a disease.

As used herein, the terms “disorder,” “condition,” or “disease” refer to cancers, and in some embodiments, associated comorbidities.

As used herein, the term “cancer” refers to any type of cancerous cell or tissue as well as any stage of a cancer from precancerous cells or tissues to metastatic cancers. For example, as used herein, cancer can refer to a solid cancerous tumor, leukemia, and/or a neoplasm.

As used herein, the term “inhibit” means to slow down or reduce the activity of a protein, enzyme, or other agent. “Inhibit” can include complete elimination of a protein or its activity. The term “inhibit” can further mean to prevent functional interaction of one or more compounds, molecules, or proteins. For example, an inhibitor can prevent a receptor from accepting its ligand or prevent activation of the receptor when accepting its ligand.

### Overview

The present disclosure concerns, in part, use of phosphatase and tensin homolog (PTEN) as a predictive biomarker to indicate responsiveness, as well as resistance, to certain therapeutic approaches for treating cancer.

Provided herein are methods for treating cancer using predictive biomarkers. Embodiments of the present disclosure include methods of treating cancer in a subject, comprising determining a PTEN status of a tumor of the subject and administering a PARP inhibitor and a PI3Kinase inhibitor to the subject if the status of the tumor is determined to be PTEN-proficient. Further embodiments include methods of treating cancer in a subject, comprising determining a PTEN status of a tumor of the subject and administering a STING agonist to the subject if the status of the tumor is determined to be PTEN-deficient (i.e., if one or more cells within the tumor is determined to be PTEN-deficient).

Also provided herein are methods of treating cancer in a subject, comprising detecting the presence of DNA double-strand break fragments associated with an exosome or a microvesicle in a tumor microenvironment and administering an immune checkpoint blockade-targeting therapy and a PI3Kinase inhibitor to the subject if DNA double-strand break fragments associated with an exosome or a microvesicle in the tumor microenvironment are detected.

### Cancer

The cancer treated by the embodiments in the present disclosure can be any cancer. In some embodiments, the cancer is melanoma, cervical cancer, breast cancer, endometrial cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplastic syndrome, multiple myeloma, transitional cell carcinoma, neuroblastoma, plasma cell neoplasms, Wilm's tumor, glioblastoma, retinoblastoma, or hepatocellular carcinoma. The cancer can be refractive to other treatments, such as chemotherapy, radiotherapy, and/or checkpoint inhibitors.

In certain embodiments, the cancer is prostate cancer. In some embodiments, the prostate cancer is advanced prostate cancer. In some embodiments, the prostate cancer is castration-resistant prostate cancer. In some embodiments, the prostate cancer is metastatic castration-resistant prostate cancer. In some embodiments, the cancer is ovarian cancer. In some embodiments, the cancer is advanced ovarian cancer.

### Treatments

PI3K inhibitors. PI3K inhibitors inhibit one or more phosphoinositide 3-kinases, leading to tumor suppression. PI3K inhibitors contemplated herein can be pan-PI3K inhibitors, or they can be specific to one or more classes or isoforms of phosphoinositide 3-kinase (for instance, beta-selective PI3K inhibitors). PI3K inhibitors contemplated by the present disclosure can include, but are not limited to, idelalisib, copanlisib (BAY 80-6946), duvelisib (IPI-145), alpelisib (BYL719), taselisib, perifosine, buparlisib (BKM120), umbralisib (TGR 1202 or RP5264), PX-866, dactolisib, CUDC-907, voxtalisib (SAR245409, XL765), ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477, and AEZS-136. In some embodiments of the present disclosure, a method of treating cancer comprises administering copanlisib.

PARP inhibitors. PARP1 is a DNA damage sensor and signal transducer that binds to DNA breaks and then synthesises poly(ADP-ribose) (PAR) chains on target proteins. These PAR chains lead to the recruitment of additional DNA repair effectors that complete the DNA repair process. Pharmacological PARP inhibitors have two general effects: catalytic inhibition of PARP1 (i.e., preventing PAR chain formation) and locking or ‘trapping’ PARP1 on damaged DNA. This blockage of repair leads to a fragmentation of the genome that ultimately kills the cell. PARP inhibitors contemplated herein can include, but are not limited to, niraparib, olaparib, rucaparib, and talazoparib. In some embodiments of the present disclosure, a method of treating cancer includes administering rucaparib.

STING agonists. Pharmacological activation of the host STING pathway via STING agonists can trigger T cell-mediated tumor regression and make immunologically ‘cold’ tumors (those that are not effectively targeted by the immune system) into ‘hot’ tumors (those that are effectively targeted by the immun system). This shift can help to overcome local immunosuppressive environments seen in cancer and can increase the effectiveness of classical immune checkpoint blockade. STING agonists contemplated herein can be either nucleotidic or non-nucleotidic. For example, STING agonists of the present disclosure include, but are not limited to, prokaryotic c-di[GMP], eukaryotic 2′,3′-cGAMP, ADU-S100 (ML-RR-S2-CDA or MIW815), MK-1454, and BMS-986301. In some embodiments of the present disclosure, a method of treating cancer includes administering BMS-986301.

Immune checkpoint blockade (ICB). Checkpoint inhibitors work by blocking immune checkpoints that shut down immune responses against cancerous cells. These molecules are able to unleash new immune responses against cancer as well as enhance existing responses to promote elimination of cancer cells. Checkpoint inhibitors are perhaps the most well-known, and most widely successful, immunomodulators developed so far. Several therapies that are currently available or in development target Cytotoxic T-Lymphocyte-Associated protein 4 (CTLA-4, also called CD152) or the Programmed Death 1 (PD-1) pathway. There is a variety of other checkpoint targets, including the following: Adenosine A2A receptor (A2AR), B7-H3 or CD276, B7-H4 or VTCN1, B and T Lymphocyte Attenuator (BTLA) or CD272, Herpesvirus Entry Mediator (HVEM), Indoleamine 2,3-dioxygenase (IDO), tryptophan 2,3-dioxygenase (TDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), V-domain Ig suppressor of T cell activation (VISTA), Sialic acid-binding immunoglobulin-type lectin 7 (SIGLEC7) or CD328, and Sialic acid-binding immunoglobulin-type lectin 9 (SIGLEC9) or CD329. The present disclosure contemplates inhibitors targeting all of these checkpoints and administration of one or more checkpoint inhibitors to a subject in need thereof. In some embodiments of the present disclosure, the checkpoint inhibitor is an antibody or antigen-binding fragment thereof. In some embodiments, the checkpoint inhibitor is a peptide. In other embodiments, the checkpoint inhibitor is a small molecule. In some embodiments, the immune checkpoint blockade-targeting therapy targets cytotoxic T-lymphocyte associated protein 4 (CTLA-4). In some embodiments, the immune checkpoint blockade-targeting therapy targets programmed cell death protein 1 (PD-1). In some embodiments, the immune checkpoint blockade-targeting therapy targets programmed death-ligand 1(PD-L1). In some embodiments of the present disclosure, a method of treating cancer comprises administering nivolumab, an antibody targeting PD-1.

### PTEN and its Use as a Biomarker for Cancer Therapies

PTEN is a potent tumor suppressor, and its loss of function is frequently observed in both heritable and sporadic cancers. PTEN has phosphatase-dependent and phosphatase-independent (scaffold) activities in the cell and governs a variety of biological processes, including maintenance of genomic stability, cell survival, migration, proliferation, and metabolism. Even a subtle decrease in PTEN levels and activity results in cancer susceptibility and favours tumor progression. Regulation of PTEN has therefore emerged as a subject of intense research in tumor biology. Recent discoveries, including the existence of distinct PTEN isoforms and the ability of PTEN to form dimers, have brought to light potential new modes of PTEN function and regulation. The status PTEN in tumors and their surroundings—i.e., whether PTEN is decreased (PTEN loss-of-function, or LOF; the tumor or subject is “PTEN-deficient”) or not (the tumor or subject is “PTEN-proficient”)—can yield information as to whether or not a patient will respond to a given therapy.

PTEN and STING agonists. PTEN-deficient cancer patients are predicted to respond to STING agonist therapy (with or without PI3K inhibitors). Studies in prostate cancer have demonstrated that concomitant castration can attenuate the anti-cancer response of PTEN-deficient cancers to STING agonist therapy. These therapies can therefore potentially be most effective if used pre-castration in either localized or locally advanced PC (versus following the development of castration-resistance in locally advanced or metastatic cancer).

In some embodiments of the present disclosure, a method of treating cancer in a subject comprises determining a PTEN status of a tumor of the subject and administering a STING agonist to the subject if the status of the tumor is determined to be PTEN-deficient (i.e., if one or more cells within the tumor is determined to be PTEN-deficient). In further embodiments, the present disclosure provides a method of treating cancer in a subject comprising the steps of: a) selecting a subject with cancer having a diagnosis of a PTEN-deficient tumor; and b) treating the subject with a STING agonist.

PTEN and PARP inhibitors. PTEN-deficient cancer patients are predicted to be resistant to PARP inhibitors (PARPi), singly and in combination with PI3K inhibitors (PI3Ki). Therefore, PTEN loss-of-function is a negative predictive biomarker for PARPi therapy, singly and in combination with PI3Ki therapy.

PTEN and PI3K inhibitors. PTEN-deficient cancer patients are resistant to single-agent PI3Ki therapy. The present disclosure provides that STING and PI3Ki in combination can control tumor growth. Present studies have demonstrated that PI3Ki in combination with STING agonist therapy effectively controls tumor growth in PTEN-deficient prostate and ovarian cancers. Given the tumor cell extrinsic mechanism of STING activation, the presence of myeloid suppressive cells within the tumor microenvironment is important for success of STING agonist/PI3Ki combination treatment.

In some embodiments of the present disclosure, a method of treating cancer comprises determining a PTEN status of a tumor of the subject and administering a PARP inhibitor and a PI3Kinase inhibitor to the subject if the status of the tumor is determined to be PTEN-proficient. In further embodiments, the disclosure provides a method of treating cancer in a subject comprising the steps of: a) selecting a subject with cancer having a diagnosis of a PTEN-proficient tumor; and b) treating the subject with a PARP inhibitor in combination with a PI3Kinase inhibitor.

PTEN and checkpoint inhibitors. PTEN-deficient cancer patients are de novo resistant to checkpoint inhibitors. The present disclosure provides that STING agonists, singly and/or in combination with PI3K inhibitors, sensitize cancer to checkpoint inhibitors. In some embodiments of the present disclosure, the methods of treating cancer include administering checkpoint inhibitors, i.e. immune checkpoint blockade-targeting therapy.

PTEN and androgen deprivation therapy (ADT). PTEN-deficient prostate cancer patients have a short-lived response to ADT relative to PTEN-proficient patients, resulting in the rapid development of castration resistance. While ADT is the standard-of-care for all advanced prostate cancer patients, the PTEN-deficient subset has limited responsiveness to ADT.

### Determining PTEN Status

The present disclosure contemplates a variety of means for determining the PTEN status of a tumor/patient, and various relevant techniques. In some embodiments of the present disclosure, determining PTEN status comprises taking a tissue biopsy from the tumor of the subject. In some embodiments, determining the PTEN status comprises next generation sequencing and/or immunohistochemistry. Next generation sequencing is characterized by highly scalability, allowing the entire genome to be sequenced at once. Usually, this is accomplished by fragmenting the genome into small pieces, randomly sampling for a fragment, and sequencing it using one of a variety of technologies. Sequencing an entire genome is possible because multiple fragments are sequenced at once (“massively parallel”) in an automated process. Immunohistochemistry (IHC) uses monoclonal or polyclonal antibodies to determine the tissue distribution of an antigen of interest in health and disease. IHC is widely used in diagnosis of cancers. Therefore, anti-PTEN antibodies are contemplated for use herein for the determination of PTEN status of a subject.

### Exosomes as a Biomarker

Exosomes are the smallest vesicles (30-100 nm) released by the fusion of multivesicular bodies containing intraluminal vesicles with the plasma membrane. Oncosomes or tumor-derived microvesicles are vesicular structures (0.1-1.0 μm) shed by outward blebbing of the plasma membrane. The presence of DNA double-strand breaks associated with microvesicles (MVs) can be utilized as a surrogate immune biomarker of response to DNA damage-induced STING pathway activation, and can predict for responsiveness to PARPi/PI3Ki combination therapy and sensitization to immune checkpoint blockade. Based on the present data, it is anticipated that administration of a PARP inhibitor, singly and/or in combination with a PI3K inhibitor, will lead to the generation/detection of DNA double-strand break (DSB) fragments associated with MVs in the blood in PTEN-proficient prostate cancers. In contrast, PTEN LOF abrogated DNA DSB association with MVs following PARPi/PI3Ki treatment, resulting in resistance to DNA damage-induced STING pathway activation. This discovery highlights the possibility of utilizing this blood-based DNA DSB/MV as a positive predictive biomarker of responsiveness to PARPi/PI3Ki and/or immune checkpoint blockade therapy. In addition, PTEN LOF can be utilized as a negative predictive biomarker for DNA damage-induced STING pathway activation.

In some embodiments of the present disclosure, methods of treating cancer in a subject include detecting the presence of DNA double-strand break fragments (DSBs) associated with one or more exosomes or microvesicles in a tumor microenvironment (TME) and administering an immune checkpoint blockade-targeting therapy and a PI3Kinase inhibitor to the subject if DNA double-strand break (DSB) fragments associated with the one or more exosomes or microvesicles in the TME are detected. In further embodiments, the present disclosure provides a method of treating cancer in a subject comprising the steps of: a) selecting a subject with cancer having DNA double-strand break fragments associated with exosomes or microvesicles in the tumor microenvironment; and b) treating the subject with an immune checkpoint blockade-targeting therapy in combination with a PI3Kinase inhibitor. In some embodiments, detecting DNA double-strand break fragments associated with one or more exosomes or microvesicles comprises taking a blood sample, or liquid biopsy, and/or a solid tissue biopsy from the subject (e.g., either from the blood or from the TME). Immunohistochemical and/or other immunofluorescent techniques (e.g., confocal staining) as decribed herein can be used, for example, to detect DSBs in the one or more exosomes or microvesicles.

In some embodiments of the present disclosure, the methods of treating cancer disclosed herein can include administration of one or more of androgen deprivation therapy, chemotherapy, and/or radiotherapy.

In some embodiments, multiple therapeutic agents (e.g., an agonist and/or an inhibitor) and/or modalities (e.g., ADT, surgery, chemotherapy, radiotherapy, etc.) can be administered to a subject at the same time, for example, in the same composition, or simultaneously but in separate compositions. In some embodiments, multiple therapeutic agents and/or modalities can be administered in series (one after another spaced in time by a period of seconds, minutes, hours, or days). Any variation of treatment methodologies is contemplated herein.

### Therapeutic Compositions

In addition to the described embodiments above, it is further contemplated that in other embodiments any therapeutic agent (e.g., a therapeutic compound, active pharmaceutical ingredient, biologic, inhibitor, agonst, etc.) described herein can be administered to a subject in need thereof either alone or in combination any other therapeutic agent disclosed herein in a therapeutic composition including one or more pharmaceutical excipients.

The therapeutic compositions of the present disclosure can take a form suitable for virtually any mode of administration, including, for example, injection, transdermal, oral, topical, ocular, buccal, systemic, nasal, rectal, vaginal, etc., or a form suitable for administration by inhalation or insufflation. Compositions that can be delivered intravenously and/or intratumorally are also contemplated herein.

Compositions containing active pharmaceutical ingredients may also contain one or more inactive pharmaceutical excipients and other substances. The therapeutic compositions described herein can include a pharmaceutically acceptable carrier, solvent, adjuvant, diluent, or combination thereof. These ingredients can include, but are not limited to, lubricants, solubilizers, alcohols, binders, controlled release polymers, enteric polymers, disintegrants, colorants, flavorants, sweeteners, antioxidants, preservatives, pigments, additives, fillers, suspension agents, surfactants (for example, anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

The therapeutic compositions described herein, or pharmaceutical compositions thereof, will generally be used in an amount effective to achieve the intended result (“effective dose” or “effective amount”), for example, in an amount effective to treat or prevent the particular disease being treated (e.g., a therapeutically effective amount) and thereby provide a therapeutic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the subject may still be afflicted with the underlying disorder. Therapeutic benefit also generally can include halting or slowing the progression of the disease.

The amount of therapeutic composition or modality administered can be based upon a variety of factors, including, for example, the particular condition being treated, the mode of administration, whether the desired benefit is prophylactic and/or therapeutic, the severity of the condition being treated and the age and weight of the patient, the genetic profile of the patient, and/or the bioavailability of the particular therapeutic composition, etc.

Determination of an effective dosage of therapeutic agents for a particular use and mode of administration is well within the capabilities of those skilled in the art. Effective dosages can be estimated initially from in vitro activity and metabolism assays. For example, an initial dosage of a therapeutic composition for use in animals can be formulated to achieve a circulating blood or serum concentration of the therapeutic composition that is at or above an EC₅₀ of the particular therapeutic composition as measured in an in vitro assay. Calculating dosages to achieve such circulating blood or serum concentrations taking into account the bioavailability of the particular therapeutic composition via the desired route of administration is well within the capabilities of skilled artisans. Initial dosages of therapeutic compositions can also be estimated from in vivo data, such as animal models. Animal models useful for testing the efficacy of the therapeutic composition to treat or prevent the various diseases described above are well-known in the art. Animal models suitable for testing the bioavailability of the therapeutic composition are also well-known. Skilled artisans can routinely adapt such information to determine dosages of particular therapeutic compositions suitable for human administration.

For any given therapeutic agent disclosed herein, dosage amounts can be in the range of from about 0.0001 mg/kg/day, 0.001 mg/kg/day, or 0.01 mg/kg/day to about 100 mg/kg/day, but can be higher or lower, depending upon, among other factors, the activity of the therapeutic agent, the bioavailability of the therapeutic composition, other pharmacokinetic properties, the mode of administration and various other factors, including the particular diseases being treated, the site of the disease within the body, the severity of the disease, the genetic profile, age, health, sex, diet, and/or weight of the subject. Dosage amount and interval can be adjusted individually to provide levels of the therapeutic compositions sufficient to maintain therapeutic and/or prophylactic effects. For example, the therapeutic compositions can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of therapeutic compositions may not be related to plasma concentration. Skilled artisans will be able to optimize effective dosages without undue experimentation.

### EXAMPLES

The Examples that follow are illustrative of specific embodiments of the disclosure, and various uses thereof. They are set forth for explanatory purposes only and should not be construed as limiting the scope of the disclosure in any way.

### Example 1

### Co-Targeting PARP and PI3K to Enhance Immune-Responsiveness in Prostate Cancer

While immune checkpoint blockade (ICB) targeting CTLA-4, PD-1/PD-L1 have shown remarkable success across cancers, most prostate cancer (PC) patients do not respond to these therapies. Therefore, there is a need to discover therapeutic strategies that enhance immune-responsiveness in ICB-refractory PC.

### Methods and Results

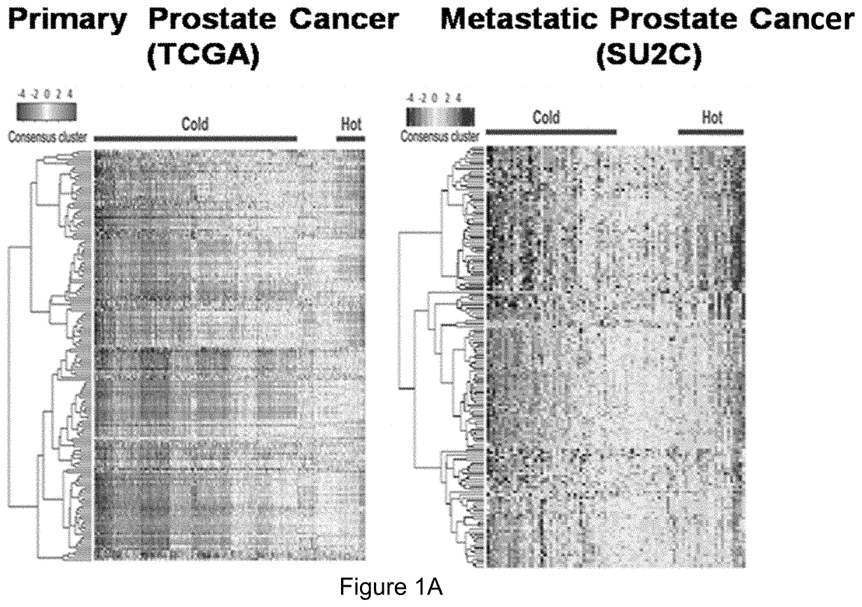

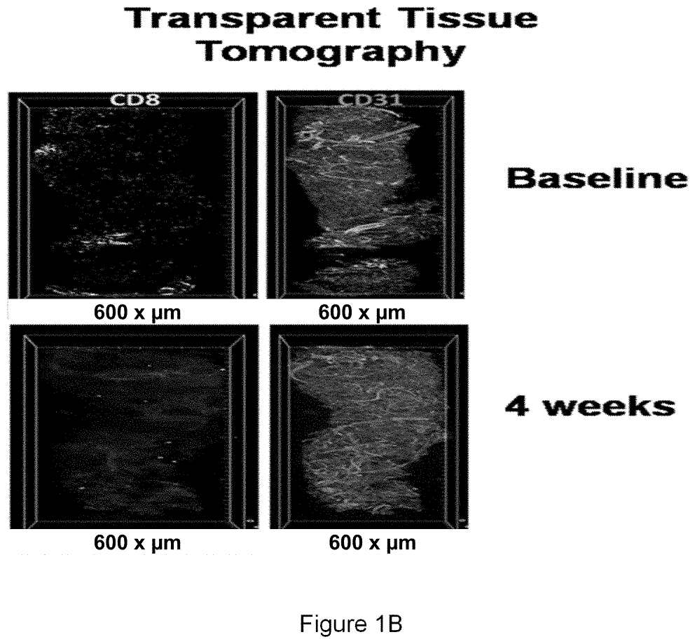

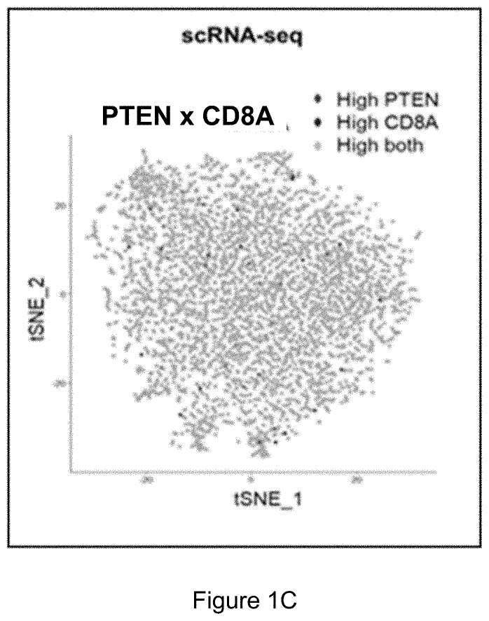

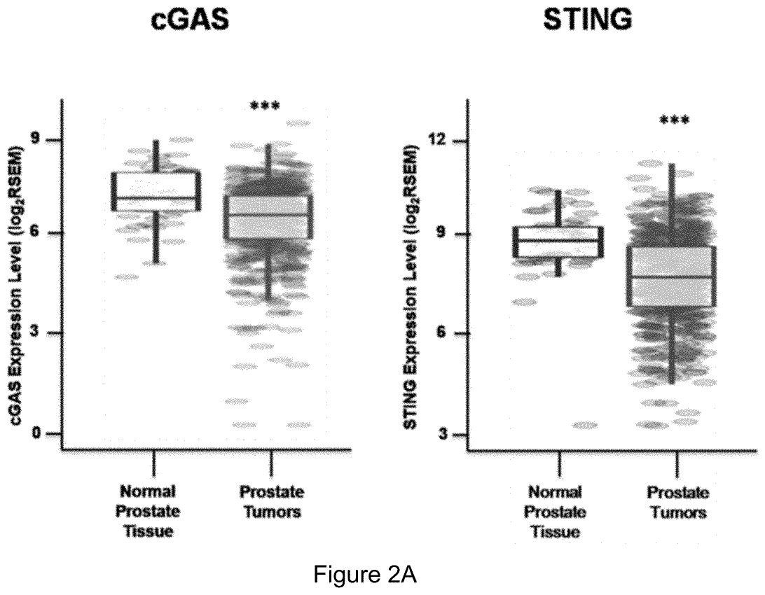

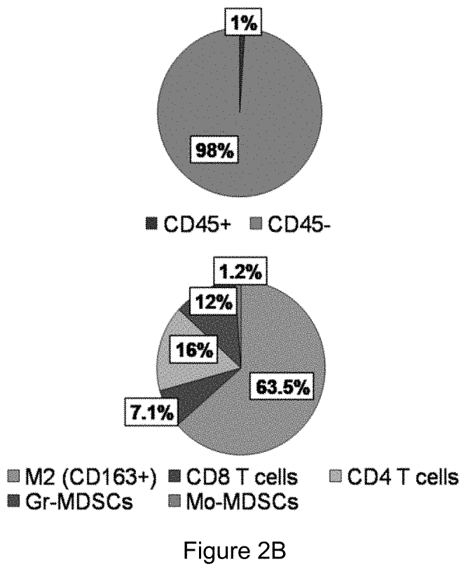

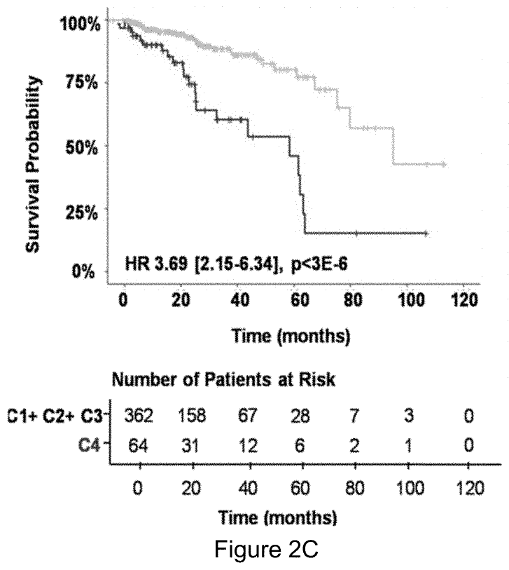

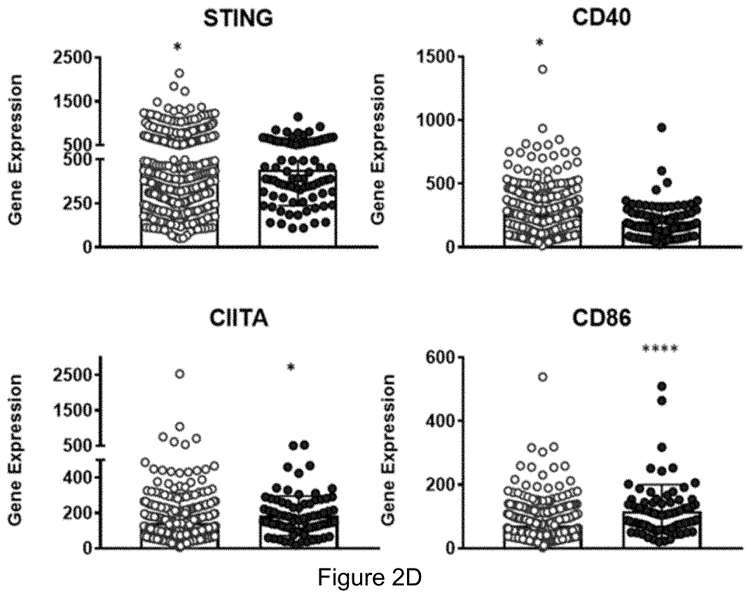

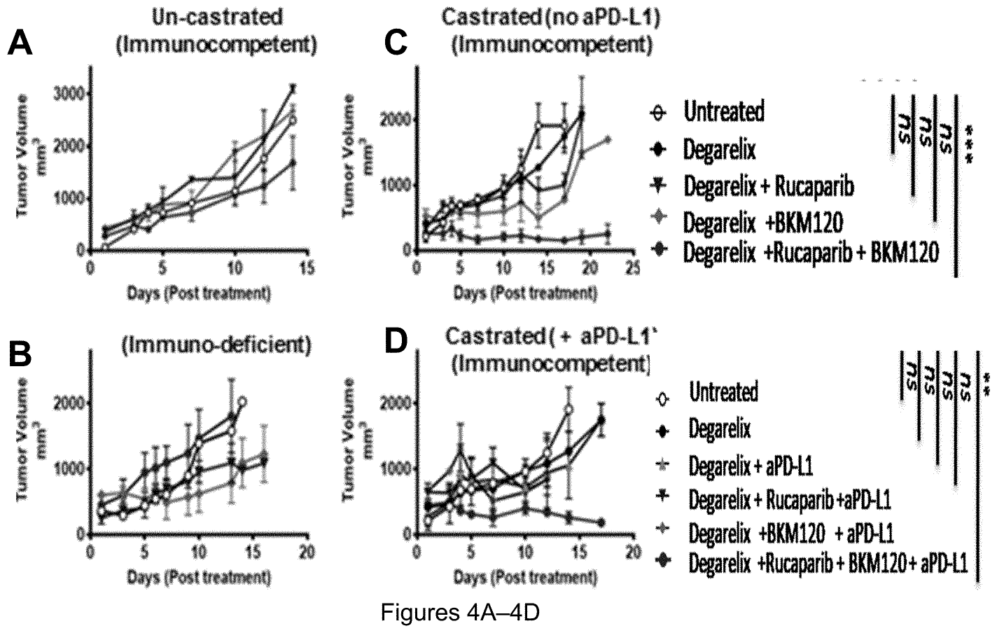

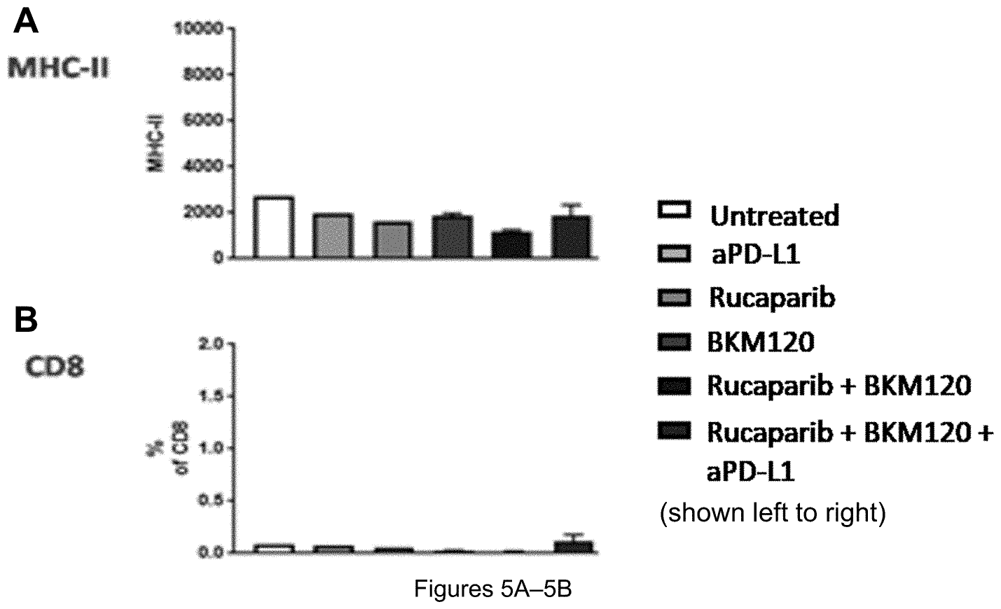

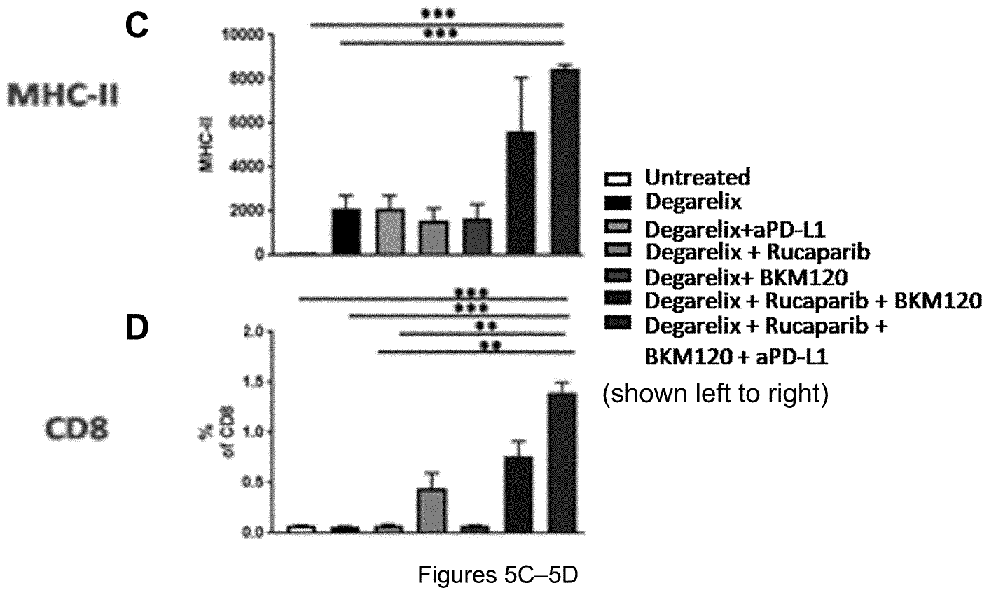

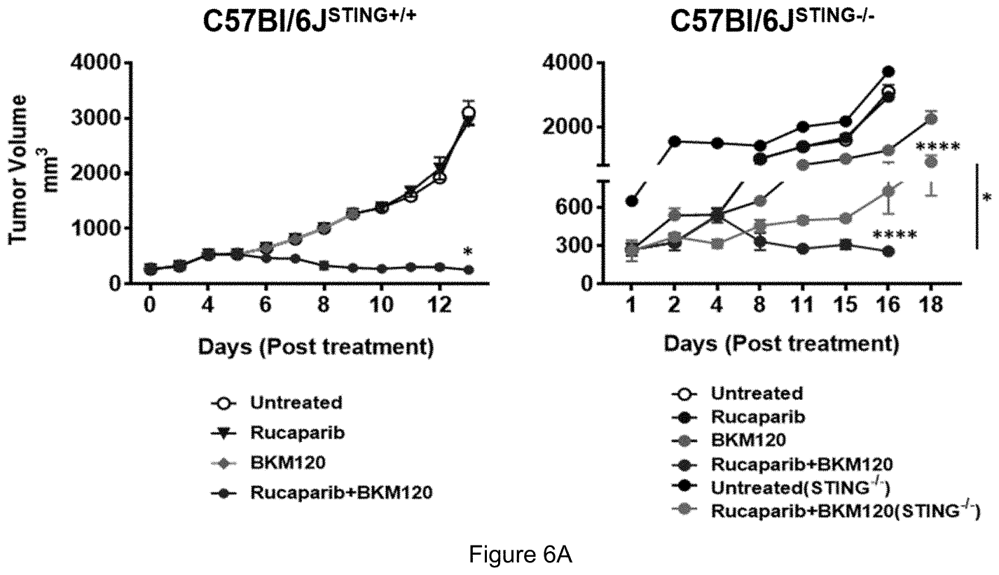

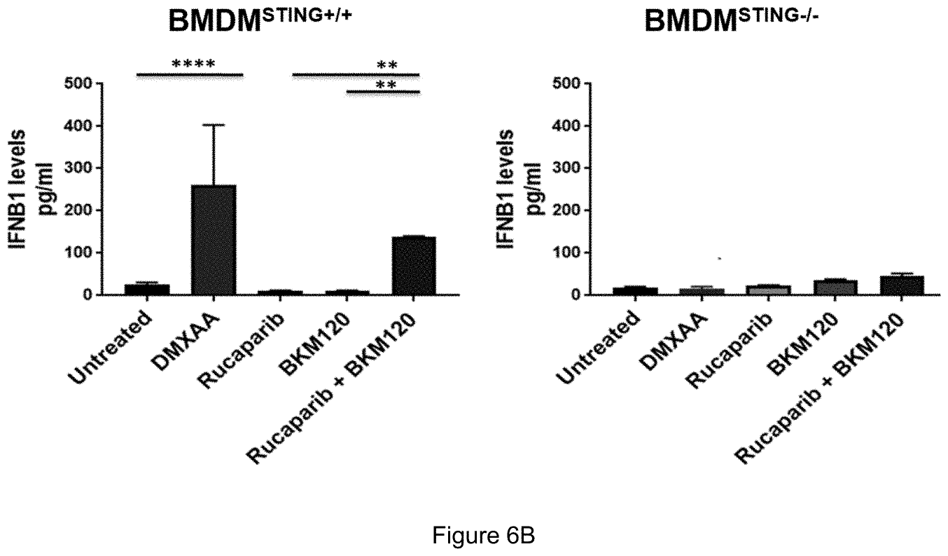

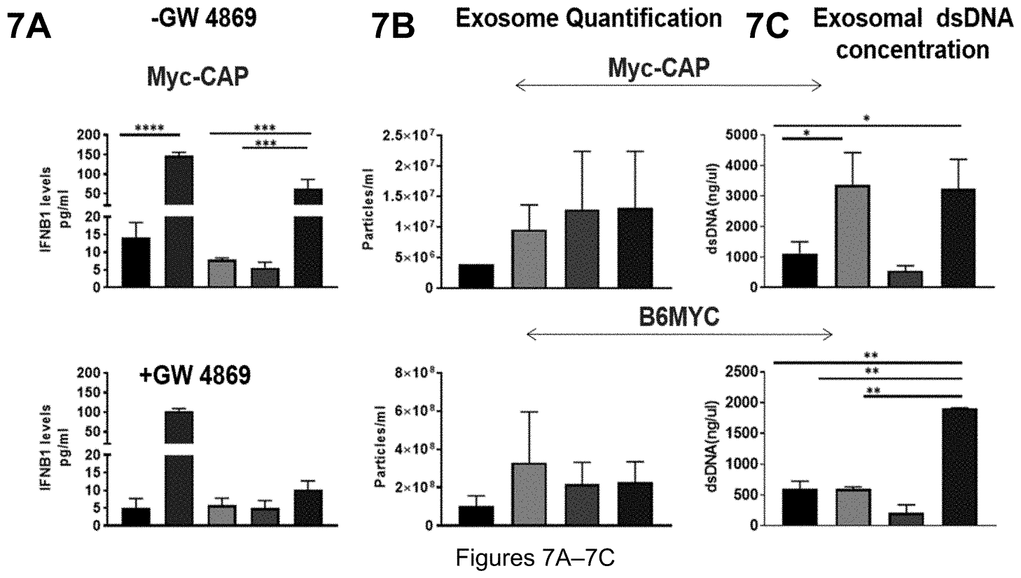

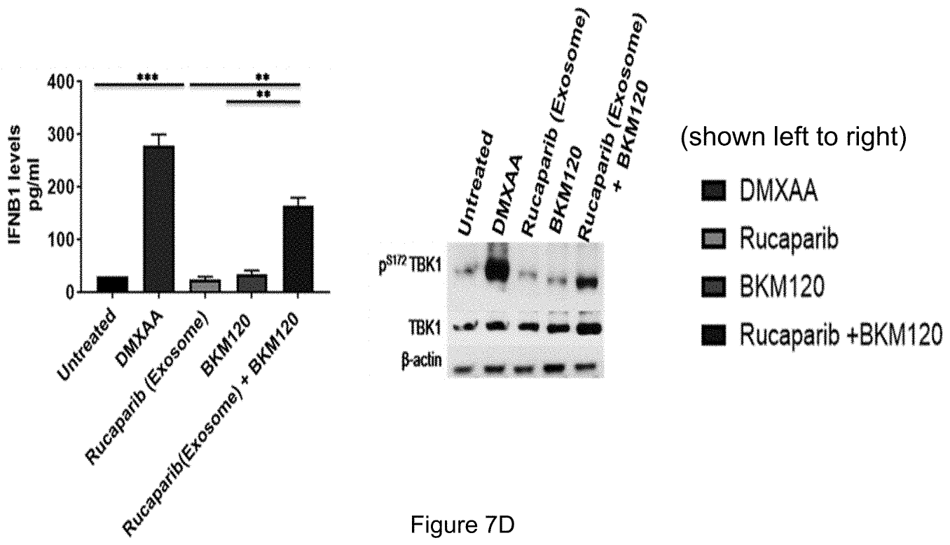

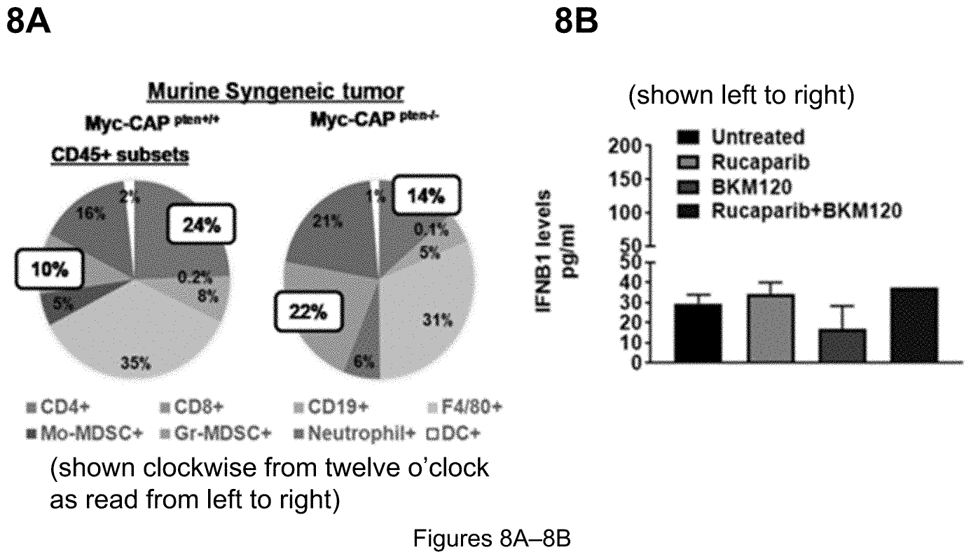

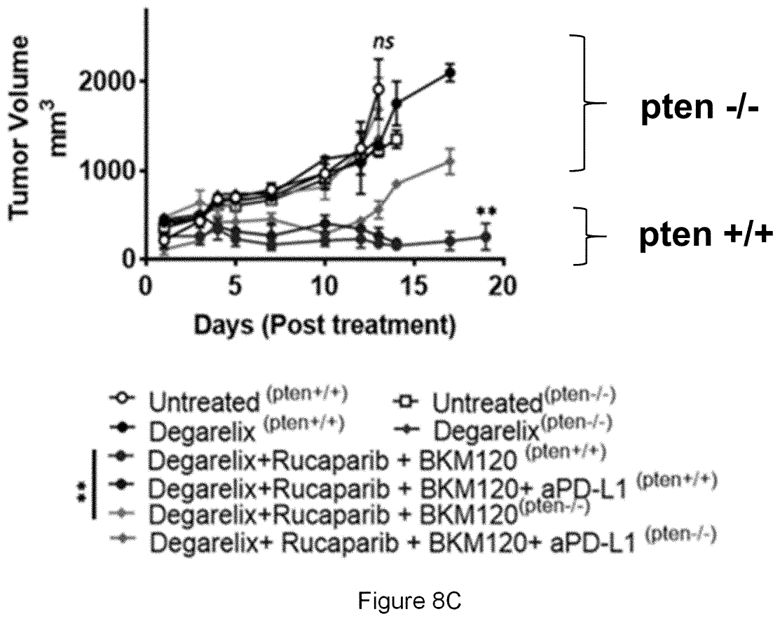

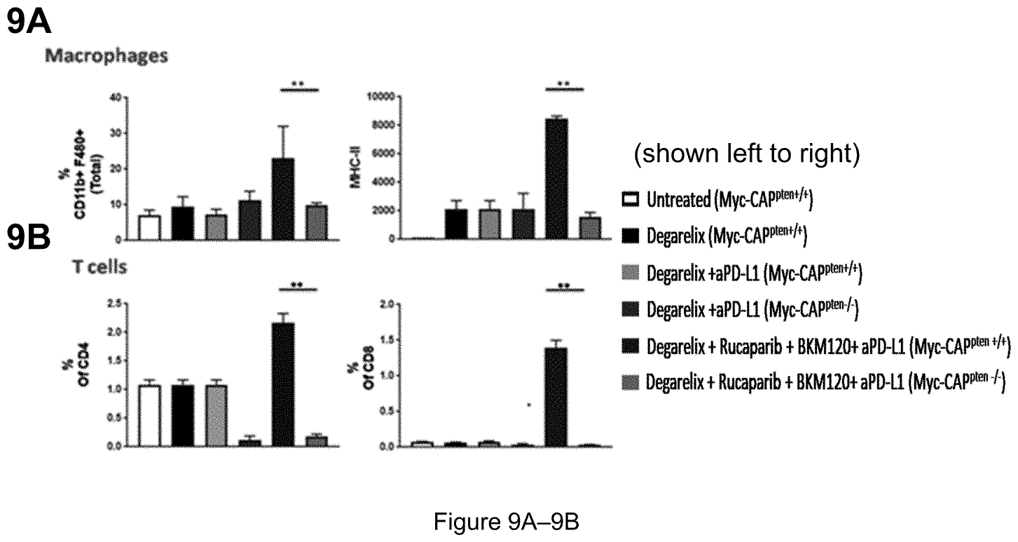

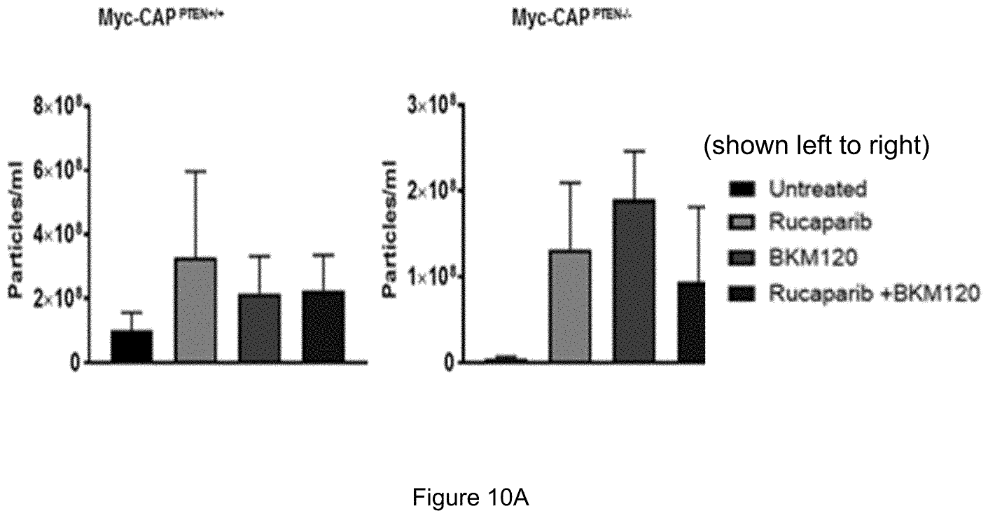

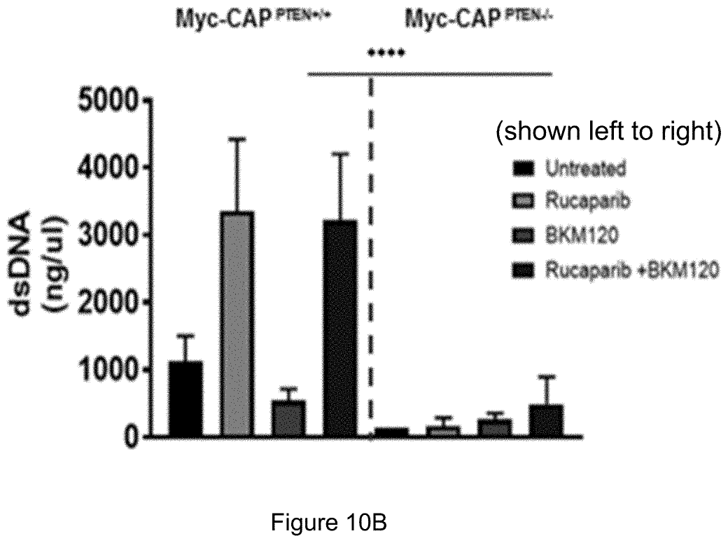

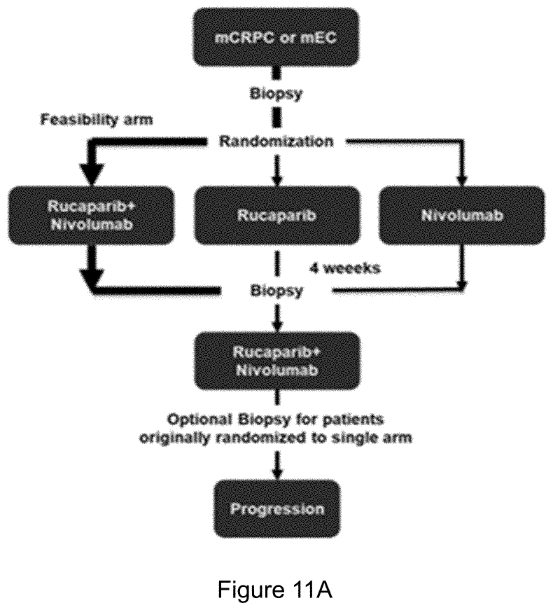

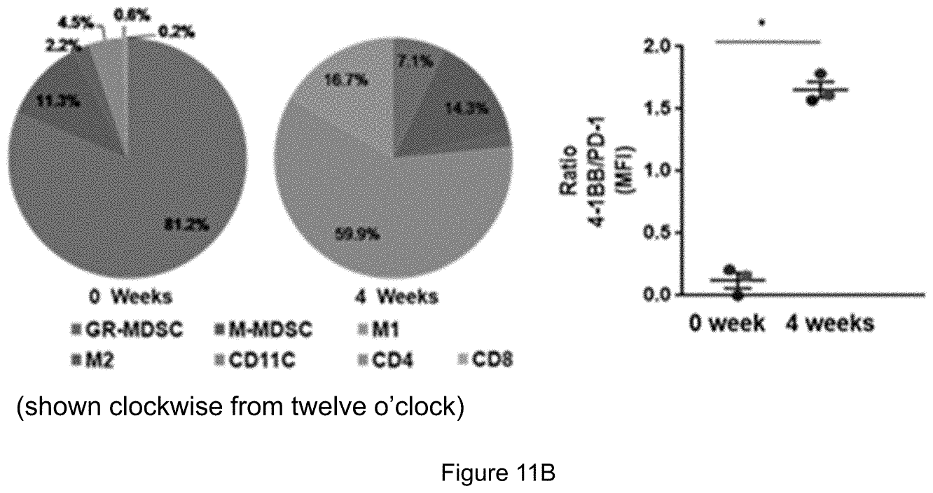

Generally in reference to FIGS. 1-12, initially, as a first step towards understanding the mechanistic basis for the general resistance of PC to ICB, gene expression data were analyzed from primary and metastatic PCs within the TCGA and SU2C, respectively, for the expression of a validated panel of 13 genes that comprise a “T cell-inflamed” gene signature. This analysis revealed that the majority of primary and metastatic PCs have a non-T cell inflamed gene expression signature. Notably, immune profiling of a representative metastatic bone marrow aspirate from a mCRPC patient that progressed on ipilimumab/nivolumab demonstrated immune exclusion within the TME. The small fraction of immune cells within the TME were dominated by myeloid immunosuppressive cells, particularly tumor-associated macrophages (TAMs) with an M2 phenotype.

Additional studies have shown that activation of the innate immune sensing cGAS/STING pathway with STING agonists can result in activation of DCs within the TME, resulting in a strong Type I interferon response, recruitment of effector T cells into the TME, and a potent anti-tumor immune response in multiple preclinical cancer mouse models. STING activation in various tumor models can occur within the host immune cells, the tumor cells, or both compartments.

As a first step towards interrogating this pathway in PC, cGAS and STING expression were assessed in primary human PC samples within the TCGA, which revealed a reduced expression relative to normal tissue counterparts. Furthermore, unsupervised hierarchical clustering of gene expression data obtained from primary PC samples within the TCGA, using previously published “low” and “high” Prostate Cancer Metastatic (PCM) signatures, revealed that patients that exhibit a poor prognosis with lower biochemical recurrence-free survival, had reduction in STING and activated M1-like myeloid gene expression, respectively. These data identify a correlation between STING expression, myeloid activation states, and overall clinical outcome.

To develop preclinical models that mimic low- vs. high-STING human prostate cancers, cGAS and STING protein expression was assessed in two transgenic myc-driven prostate cancer lines, myc-CAP and B6-myc, generated in FVB and C57BL6 genetic backgrounds, respectively. Low cGAS/STING expression occurred in myc-CAP and high cGAS/STING expression occurred in B6-myc cells.

Thus, myc-CAP and B6-myc cells can serve as models of STING-low and STING-high human PC, respectively. It was previously shown that a combination of PARP inhibitors (PARPi) and pan-PI3K inhibitors (PI3Ki) induces additive DNA damage in PC cells. While treatment of both myc-CAP and B6 myc cell lines with PARPi/PI3Ki resulted in additive DNA damage in vitro, tumor regression in vivo was only observed in syngeneic B6-myc (high-STING), but not myc-CAP (low STING) tumors, suggesting that activation of tumor cell-intrinsic STING signaling in response to DNA damage is critical for optimal anti-tumor responses.

Prior studies have demonstrated that early stages of castration induce T-effector and T-regulatory cell infiltration within prostate tumors. Therefore, the hypothesis that immunostimulatory effects of castration would synergize with PARPi/PI3Ki combination, which enhances DNA sensing STING pathway activation, was tested to elicit a potent anti-tumor response in syngeneic myc-CAP tumors. The combination of degarelix and PARPi/PI3Ki resulted in suppression of tumor growth in myc-CAP (low-STING) tumors, which was not observed with degarelix alone or PARPi/PI3K treatment. Importantly, the anti-tumor response observed with castration/PARPi/PI3Ki was abolished in immunodeficient nude mice, suggesting that this regimen induces tumor control via an immune-dependent mechanism. Furthermore, PARPi/PI3Ki/ADT led to increased macrophage infiltration/activation and CD8 T cell infiltration within myc-CAP tumors, and this was enhanced by ICB. Notably, PTEN-deficient myc-CAP tumors were observed to have increased infiltration of myeloid cells, specifically tumor-associated macrophages (TAMs) and granulocyticmyeloid derived suppressor cells (Gr-MDSCs), and decreased infiltration of CD4, CD8 and dendritic cells within the TME, relative to their PTEN-proficient counterparts. These findings were corroborated by pooled bioinformatic analysis using PTEN-deficient primary and metastatic prostate cancer samples from the TCGA and SU2C, which demonstrated a significant enrichment of MDSC-high tumors within PTEN-deficient prostate cancers.

Next, the therapeutic impact of the PARPi/PI3Ki/ADT combination in a myc-CAP-PTEN deficient syngeneic model, generated by CRISPR/CAS9-mediated deletion of PTEN, was tested. Surprisingly, the combination of PARPi/PI3Ki/ADT fails to enhance tumor macrophage infiltration/activation and CD8/CD4 T cell infiltration within myc-CAP/PTEN deficient tumors, resulting in a lack of anti-tumor response (not shown). Based on these results, it was postulated that PTEN deficient-prostate cancers are de novo resistant to immunotherapy, through dysregulation of tumor cell intrinsic and extrinsic DNA-sensing STING pathway activation within the TME, and this resistance can be overcome by STING agonist-based IO combinations.

As a first step towards testing this hypothesis, ex vivo co-culture assays were performed of PTEN-proficient myc-CAP PC cells with bone-marrow derived macrophages (BMDMs), which revealed that PARPi/PI3Ki treatment increased macrophage activation in a STING-dependent manner, and this myeloid reprogramming was mediated by microvesicles or exosomes carrying dsDNA released from drug-treated tumor cells.

Recent work has shown that AKT can phosphorylate c-GAS at Ser-291 or Ser-305, leading to post-translational suppression of its enzymatic activity. It was hypothesized that PI3Ki treatment can derepress c-GAS enzymatic activity, in addition to inducing additive DNA damage in combination with PARPi. To test this hypothesis, the impact of microvesicles (MVs) collected from PARPi-treated myc-CAP cells, and then treated BMDMs with MVs alone, PI3Ki alone, or the combination of MVs/PI3Ki was assessed. Strikingly, the combination of MVs/PI3Ki resulted in STING activation within BMDMs, similar to what was achieved with direct treatment of BMDMs with STING agonists. In contrast, MVs derived from PARPi-treated myc-CAP/PTEN-null cells, in combination with PI3Ki, were unable to activate STING within macrophages. These preliminary observations were corroborated in ex vivo studies utilizing prostate tumors from PTEN-deficient GEMMS, which revealed an inability of PARPi/PI3Ki to drive c-GAS/STING pathway activation and Type I interferon production within myeloid cells. Importantly, it was observed that MVs released from PTEN-deficient PC cells lack dsDNA, thus providing one potential mechanism of resistance to DNA sensing STING pathway activation.

For in vitro work, all isogenic cell lines were grown in DMEM medium containing 10% FBS, 1% P/S, and 2% L-glutamine. Bone marrow derived macrophages (BMDM) from wild type and STING−/− mice used for co-culture experiments were differentiated in 10% RPMI containing 1% P/S and 30% L-conditioned media for a total of 5 days. For immune profiling by flow cytometry, cells were stained using the following antibodies: anti-mouse CD45, CD11b, F480, MHC-II, CD86, PD-L1, PD-1, CD4, and CD8. The supernatants from the co-culture experiments were utilized for quantification of IFNB1 (Biolegend) and related cytokines by ELISA. To look for biomarkers of DNA damage and signaling proteins downstream of STING activation, confocal staining for cytosolic dsDNA and western blot for polyADP-ribosylation, p-gamma-H2AX, cGAS/STING, pIRF3/IRF3, pTBK1/TBK1, and GAPDH was completed on cell lines. Exosomes were isolated by ultracentrifugation and utilized for quantitative and qualitative analysis. For in vivo experiments, Myc-CAP cells were engrafted subcutaneously in FVB mice and B6-MYC tumor explants were implanted into C57Bl/6J mice. Statistical analysis was done by one-way ANOVA.

### Discussion

Treatment of c-myc-driven PC cell lines in vitro with PARPi/PI3Ki combination results in increased DNA damage and cytosolic release of DNA fragments. Ex vivo and in vitro co-culture assays showed that DNA damage in cancer cells induces STING-dependent activation of tumor-associated macrophages, resulting in phenotypic shift of suppressive M2 cells into M1 macrophages, expressing high levels of MHC-II and enhanced antigen presentation capacity. Interestingly, STING activation within myeloid suppressive cells induced by the PARPi/PI3Ki combination was mediated by dsDNA-containing exosomes released by PARPi-treated tumor cells, and PI3Ki-mediated de-repression of c-GAS enzymatic activity within macrophages. Exosome reconstitution assays demonstrated that DNAse treatment of the microvesicles isolated from PARPi/PI3Ki-treated cell lines completely abolished type I interferon response elicited within macrophages. The efficacy of PARPi/PI3Ki combination was tested in two independent c-myc^{hi} in vivo models of PC, where significant inhibition of tumor growth was observed. Flow cytometric analysis of single cell suspensions from tumor showed increased infiltration of activated macrophages. Strikingly, exosomes from PARPi-treated PTEN−/− tumor cells (which undergo DNA damage) were unable to activate STING within macrophages, due to absence of dsDNA fragments within exosomes, consistent with the absence of macrophage activation and CD8 infiltration observed in PTEN-deficient mychi models in vivo. This block in DNA damage-induced STING pathway activation within PTEN-deficient cancers can be overcome by administration of direct STING agonists in vivo.

### Conclusions

These results demonstrate that DNA damage elicited by PARPi/PI3Ki can reprogram the myeloid tumor microenvironment to enhance anti-tumor immunity within PTEN+/+ cancers. The block to DNA damage-induced STING pathway activation in PTEN−/− cancers can be overcome by administration of direct STING agonists in vivo. Additional experiments are needed to evaluate differential immunologic and therapeutic impact of PARPi/PI3Ki or STING agonist/PI3Ki combinations on sensitization of PTEN+/+ and PTEN−/− PCs to ICB.

PARPi/PI3Ki results in STING activation in the myeloid microenvironment in myc-driven murine prostate models. STING activation within myeloid suppressive cells was mediated via dsDNA-containing exosomes released from tumor cells. PI3Ki-mediated de-repression of c-GAS activity within macrophages was required for optimal STING pathway activation induced by DNA damage. The combination of PARPi/PI3Ki enhanced T cell infiltration, resulting in tumor regression of myc-driven murine PC in vivo. Preliminary immune profiling of metastatic biopsies demonstrated relief of immune suppression in patients treated with Rucaparib/Nivolumab clinical trial. PTEN loss is a negative predictive biomarker for DNA damage-induced STING pathway activation.

PTEN-deficient tumors are resistant to DNA damage-induced STING pathway activation, as determined by using PARPi in combination with PI3Ki. PTEN loss is therefore a negative predictive biomarker for DNA damage induced STING pathway activation. This discovery could explain emerging clinical trial data that PTEN loss (despite inducing homologous recombination deficiency) has NOT been shown to be a positive predictive biomarker to PARPi therapy in patients with metastatic, castrate resistant prostate cancer.

STING agonists are being deployed to ask the question of whether direct STING agonism within the tumor microenvironment (TME), not DNA immunogen-induced, can bypass the dependency on dsDNA and induce an anti-tumor response in PTEN-deficient prostate cancer murine models. Data obtained using DMXAA (a mouse STING agonist) demonstrate that PTEN-deficient prostate cancers are resistant to direct STING agonists as well.

### Example 2

### PARP and PI3K Inhibitor Combination Therapy Eradicates c-MYC-Driven Murine Prostate Cancers Via cGAS/STING Pathway Activation within Tumor-Associated Macrophages

### Abstract

The majority of metastatic, castrate-resistant prostate cancer (mCRPC) patients are de novo resistant to immune checkpoint blockade (ICB), so therapeutic strategies to enhance immune response are urgently needed. In this study, a co-clinical trial was performed of PARP inhibitor (PARPi) in combination with PD-1 or PDL-1 antibody in genomically unselected mCRPC patients or homologous-recombination proficient murine models, respectively, which demonstrated lack of efficacy. In contrast, PARPi in combination with PI3K inhibitor (PI3Ki), induced tumor regression via macrophage STING-dependent innate immune activation in vivo, and enhanced T-cell infiltration/activation in c-myc driven murine prostate cancer models, which was augmented by PDL-1 blockade. Ex vivo mechanistic studies revealed that PARPi-induced DNA double strand break-associated microvesicles released from tumor cells, coupled with PI3Ki-mediated c-GAS de-repression, were both required for macrophage cGAS/STING pathway activation. These data demonstrate that the PARPi/PI3Ki combination triggers macrophage STING-mediated anti-cancer innate immunity, which is sufficient to induce tumor regression in ICB-refractory c-myc-driven prostate cancer.

### Statement of Significance

Co-targeting of PARP and PI3K signaling pathways activates the c-GAS/STING pathway within macrophages, thereby enhancing T cell recruitment/activation into the tumor microenvironment and cancer clearance in c-myc-driven murine models. PARPi/PI3Ki combination therapy could markedly increase the fraction of mCRPC patients responsive to ICB, independent of germline or tumor homologous recombination status.

### Introduction

Prostate cancer (PC) is the most common malignant neoplasm in men and the second most frequent cause of cancer death for males in the United States. While there have been incremental advances, mCRPC remains an incurable disease with high morbidity and mortality, so there is an urgent need to develop definitive treatments that improve survival. Over the past decade, there has been a resurgent interest in cancer immunotherapy, partly based on the profound and durable clinical responses to immune checkpoint blockade (ICB) antibodies targeting CTLA-4 and PD-1/PD-L1. However, only approximately 10-25% of mCRPC patients respond to these approaches.

MYC is a “master” proto-oncogene that contributes to tumorigenesis of greater than 75% of all advanced, refractory human cancers, particularly prostate, colon, breast, cervical cancers, acute myeloid leukemia, lymphomas, small-cell lung cancer, and neuroblastoma. C-myc is a transcription factor encoded by the MYC gene on locus 8q24.21, which is frequently amplified in human cancers. However, despite multiple pharmaceutical efforts, c-myc has remained “undruggable.” Furthermore, c-myc driven-cancers are resistant to ICB. Therefore, therapeutic strategies that target c-myc-driven cancers and enhance their responsiveness to ICB are urgently needed.

The cGAS/STING pathway is known to be physiologically activated by cytosolic double-stranded DNA (dsDNA), which typically occurs in the context of viral infections, resulting in the generation of cytosolic cyclic dinucleotides generated by the Cyclic GMP-AMP synthase (cGAS) enzyme, downstream activation of the Stimulator of Interferon Genes pathway and induction of Type I interferon (IFN) production. Recent preclinical studies have demonstrated that PARPi, which are FDA approved for BRCA ½ mutated prostate, breast, ovarian, and pancreatic cancers, can activate the innate immune cGAS/STING pathway in murine homologous recombination (HR)-deficient breasts and ovarian cancer models, resulting in sensitization of these tumors to ICB. Furthermore, preclinical data suggests that PARPi can elicit DNA damage in HR-proficient cancers, but this response is generally insufficient for meaningful clinical activity.

To test the hypothesis that PARPi and resulting DNA damage can sensitize HR-proficient mCRPC to ICB, a co-clinical trial was performed testing the combination of PARPi with PD-1 or PD-L1 antibody in both HR-proficient mCRPC patients and murine models, respectively, which demonstrated lack of efficacy. In contrast, concomitant PI3K inhibitor (PI3Ki) treatment with PARPi induced tumor regression in c-myc driven murine PC models via tumor cell-extrinsic, macrophage STING-dependent innate immune activation, which was accompanied by enhanced T-cell infiltration/activation. Critically, the anti-tumor response elicited by PARPi/PI3Ki was augmented by PD-1/PD-L1 axis blockade and abrogated in immunodeficient mice and immunocompetent mice treated with systemic macrophage depleting agent (Clodronate) or STING antagonist (H-151). Mechanistically, it was observed that DNA double-strand break (DSB)-associated MVs released from PARPi-treated transgenic c-myc-over-expressing cancer cells, along with concomitant PI3Ki-mediated post-translational de-repression of cGAS enzymatic activity, increased cGAMP and activated the STING pathway within tumor-associated macrophages (TAMs). Taken together, these data demonstrate that the PARPi/PI3Ki combination drives anti-cancer innate immunity via cGAS/STING pathway activation within TAMs, resulting in tumor regression in murine models of c-myc-driven PC. Based on these observations, clinical trials testing PARPi/PI3K inhibitors with ICB are warranted in immunotherapy-refractory HR-proficient advanced PC.

### Methods

Rucaparib/nivolumab clinical trial in mCRPC patients. mCRPC prostate cancer patients, independent of HR status, who had received at least one AR targeted therapy, without prior exposure to PARPi or ICB therapy, were enrolled in an investigator-initiated, IRB-approved co-clinical trial (NCT03572478) of rucaparib (PARPi) with nivolumab (PD-1 antibody) and treated until disease progression or unacceptable toxicity. All patients provided informed consent prior to clinical trial enrollment. As part of study requirements, serial PSAs were obtained on a monthly basis following study enrollment and measured using standard clinical laboratory diagnostic methods.

Multi-Parameter Flow Cytometry. Human Biopsies: Tissues were processed into single cell suspensions via gentle mechanical dissociation in 12-well plates containing 1 ml of 10% RPMI media supplemented with 10% fetal bovine serum, 1% penicillin-streptomycin and 2% L-glutamine. Cell suspensions were centrifuged at 500 g for 5 minutes at 4° C., resuspended in FACS buffer (1× PBS containing 0.5% FBS and 0.01% sodium azide) and used for staining with the following anti-human antibodies (Biolegend®): CD45, CD11b, CD163, CD68, HLA-DR, CD15, CD33, CD16, PD-L1, CD3, CD4, CD8 CD19, 4-1BB, PD-1, CD11c. All flow antibodies in this study were utilized at recommended dilutions provided by the manufacturer. Murine tumors: Murine tumors were processed identically with an additional step of filtration to remove cell debris, where single cells were passed through a 70-micron mesh, prior to stain. One million cells resuspended in 1× FACS buffer were stained with titrated concentrations of the following anti-mouse antibodies: CD45, CD11b, CD11c, CD19, F480, Ly6G, Ly6C, PD-L1, and VISTA, I-Ae/IAb, H-2^{Kb}, CD3, CD4, CD8, 4-1BB, PD-1, CD206, and CSF-1R. Incubation with antibodies was done at 4° C. for 30-40 minutes for both murine and human cells. Following staining, cells were washed twice with 1× FACS buffer and fixed with 300 μl of 4% paraformaldehyde (Fisher Scientific®), prior to analysis on BD instrument LSR 4-15 Fortessa™. Data collected on flow cytometer using BDFACSDIVA software and was analyzed using FlowJo® software (Tree Star).

Bioinformatics analysis of myeloid gene signature using TCGA database. The transcriptome data (Illumina HiSeq RNASeqV2) was downloaded for prostate cancer tumors and normal prostate from the TCGA data portal and analyzed for differential expression of STING and c-GAS. Additionally, the Biochemical Recurrence (BCR) status and Gleason Scores were also downloaded for 488 prostate tumors. The samples were grouped into High Gleason score (≥8) and Low Gleason score. The RNA-Seq data was used to analyze the differential expression of genes between High and Low Gleason score samples. Statistical analysis for evaluating changes in gene expression between the different groups were done using unpaired non-parametric t-test.

Cancer Cell Lines. Transgenic c-myc” prostate tumor derived cell line, Myc-CAP) (STING^{lo}) was obtained from ATCC and passaged in 1× DMEM (without phenol red) containing 10% fetal bovine serum, 1% penicillin-streptomycin and 2% L-glutamine. The corresponding c-myc^{hi} line derived from a C57BL6 generated background were grown for in vitro studies, using same culture conditions as for Myc-CAP. All cell lines were confirmed to be Mycoplasma-free using the Universal Mycoplasma Detection Kit (ATCC® 30-1012K™) testing kit. Both the B6-Myc (STING^{hi}) cell line and the B6-Myc whole tumor explants used for in vivo studies were a kind gift from Dr. Leigh Ellis (Dana Farber Cancer Institute, Boston). For in vitro drug treatments, the following concentrations were used: Rucaparib (500 nM), Buparlisib (1 μM), DMXAA (50 μg/mL) with specific treatment durations for individual experiments indicated in the figure legends.

Western Blot Analysis. RIPA and T-PER buffer (Thero Scientific®), supplemented with protease (Roche®) and HALT phosphatase inhibitor cocktail (Roche®), were used for preparation of lysates from in vitro cell lines and whole tumor chunks, respectively. For western blotting, the following antibodies were used from Cell Signaling Technology®: Polyclonal rabbit anti-mouse-phospho-γH2AX, phospho-AKT, total AKT, cGAS, STING, phospho-IRF3, total IRF3, phospho-TBK1, total TBK1, PTEN, β-actin and GAPDH. Monoclonal anti-mouse PAR antibody was obtained from Trevigen®. Images of scanned blots were processed using ADOBE Photoshop®.

Generation of BMDMs. Bone marrow derived macrophages were differentiated as previously described. Briefly, bone marrow cells were isolated from male FVB/NJ, C57Bl/6J^{STING+/+} and C57BL/6J-Sting1^{gt}/J^{(STING-/-)} mice and differentiated in the presence of 10× RPMI media (supplemented with 10% fetal bovine serum, 1% penicillin-streptomycin and 2% L-glutamine) containing 30% L-conditioned media or M-CSF (50 ng/ml) for 5-7 days. Differentiated cells were stimulated directly with 50 μg/ml of 5,6-Dimethylxanthenone-4-acetic Acid (DMXAA, mouse STING agonist) for 36 hours. Following treatment, supernatants were collected for Type I IFN ELISA (LEGEND MAX™ Mouse IFNβ1 ELISA, Biolegend®) and processed as specified in protocol.

Generation of syngeneic models and in vivo drug administration. Wild-type (WT) C57BL/6J, C57BL/6J-Sting1^{gt}/J^{(STING-/-)}, FVB/NJ mice, Athymic nude (Nu/J) and NOD-SCID (NOD.CB17-Prkdc/J) were purchased from Jackson laboratories and mice were kept in an AALAC (American Association for the Accreditation of Laboratory Animal Care) certified barrier facility at the University of Chicago. Animal work was carried out according to approved Institutional Animal Care and Use Committee protocols. For Myc-CAP-based experiments, mice aged 8-10 weeks were engrafted with 1 million Myc-CAP cells re-suspended in 1× PBS, under anesthesia. For experiments using B6-Myc, 5 mm² tumor chunks were implanted subcutaneously in mice. Treatments were started when tumor volumes reached approximately 200-400 mm³, and mice were randomly allocated to treatment groups as indicated. For in vivo treatments, lyophilized drugs were reconstituted in appropriate solvents and were administered at the following doses: Degarelix (0.625 mg/kg) was administered as a single intraperitoneal (i.p.) injection. Rucaparib (Clovis Oncology®) and buparlisib (the Stand up to Cancer Drug Formulary at Dana Farber Cancer Institute) were administered daily by oral gavage at 150 mg/kg and 30 mg/kg, respectively, whereas anti-mouse PD-L1 (clone 10F.9G2; BioXcell®) was administered i.p. at 100 μg once every 2 days. DMXAA was injected intratumorally once at a dose of 500 μg/kg. Exosomal Inhibitor GW4869 (Sigma Aldrich®) and STING antagonist H-151 (Invivogen®) were dosed at 500 μg/gm of body weight i.p. daily and 750 nanomoles/kg i.p. daily, respectively. For in vivo macrophage depletion studies, Clodronate (Standard Macrophage Depletion kit, Encapsula Nanosciences) was injected i.p. on a weekly basis at recommended dose of 300 μL of clodronate-liposomal emulsion containing 18.4 mM concentration of clodronate). All in vivo treatments were done for 15-28 days and tumor volume measurements were collected on a daily basis. Tumor volume was calculated using the formula: 0.5×longest diameter×(shortest diameter)². Euthanasia was performed for mice bearing tumor ulceration and/or tumor diameter >2 cm, as per IACUC-approved protocol.

Confocal Microscopy. Tumor cells were grown at titrated seeding density in glass bottom plates and treated with indicated drug(s) at concentrations described above. Following 36 hours of treatment, culture media was aspirated and the cells were washed twice with 1×PBS. Cells were then fixed with 4% paraformaldehyde at 4° C., followed by permeabilization briefly with cold 100% ethanol for 8 mins at 4° C. Staining for DNA DSBs was done with anti-mouse primary antibody specific for phospho-γH2AX (1:500 dilution, Cell Signaling Technology and secondary anti-rabbit IgG antibody conjugated to AF647 (1:1000-1:2000 dilution in 1×PBS, Thermo Fisher Scientific®). Anti-mouse specific β-actin conjugated to Phycoerythrin (PE, Thermo Fisher Scientific®) was used to stain the cytoskeleton. All staining procedures were done at 4° C. for 30 minutes. Cells were then washed 3 times with 1×PBS and imaged immediately. All images were collected using an Olympus Fluoview® 1000 using a 100× oil objective. Acquired images were analyzed by ImageJ software, developed at NIH.

MV Isolation/DNA extraction. Cells were treated for 36 hours with the indicated drug(s), and supernatants were harvested and then centrifuged at 300×g for 5 minutes at 4° C. to pellet cells. This was followed by additional centrifugation steps at 2,000×g for 10 min at 4° C. to eliminate dead cell debris and at 10,000×g for 30 min in at 4° C. to remove larger vesicles. The supernatant was then collected and subjected to 100,000×g centrifugation in a Type 60 Ti rotor (38000 rpm) for 70 min at 4° C. The 100,000×g pellet was suspended in 1×PBS to the initial volume of supernatant (2 mL), and washed by an additional spin in the ultracentrifuge for 70 min at 4° C. The final MV pellet was collected in 1×PBS and used for quantification. Measurements of particle size distribution (PSD) and concentration were performed with a Nanosight® LM10 HS-BF instrument (Nanosight Ltd, UK), based on NTA measurements, using a 405-nm 65-mW laser and an EMCCD Andor® Luca camera and revealed MV size range of 50-100 nm.

Samples were diluted with particle-free PBS in a 1:100 dilution (pH=7.4) to reach the optimal concentration for NTA. All measurements were performed under the identical camera settings (Shutter: 850, Gain: 450, Lower Threshold: 910, Higher Threshold: 11180, 60 s) and processing conditions (NTA 2.3 build 0033, Detection Threshold: 9 Multi, Min Track Length: Auto, Min Expected Size: minimum of 30 nm). Measurements were performed in multiple repeats (n=3) to collect an at least 5000 events, and then equal numbers of MVs were used for downstream assays. Equal numbers of MVs were collected from each treatment group and DNA was extracted using Trizol® LS as per protocol (Thermo Fisher Scientific®), and then quantified using Nanodrop®.

Viability Assays. Single cell suspensions at a concentration of 0.5 million cells/0.5 mL of 1× Annexin Buffer, were stained with Annexin V/PI (FITC Annexin V Apoptosis Detection kit, BD Biosciences) as specified in manufacturer protocol. Acquisition and analysis of the data sets were done as previously described in section on Multi-parameter flow cytometry.

Ex vivo reconstitution assay. Subcutaneous tumors were isolated by gentle mechanical dissociation in the presence of 10×RPMI (supplemented with 10% fetal bovine serum, 1% penicillin-streptomycin and 2% L-glutamine). 1×ACK was used for RBC lysis. Single cell suspensions were washed twice with media, by centrifuging at 500×g for 5 minutes at 4° C. and quantified using 0.1% Trypsin solution. For sorting of CD45+ cells, PE selection kit (EasySep™ Mouse PE Positive Selection Kit) was used for staining and magnetic extraction of positively labeled cells, as per protocol from vendor. Single cell suspensions derived from tumor were seeded at a concentration of 0.2-0.5 million cells/mL and treated with the following drugs: DMXAA (50 μg/mL) rucaparib 500 nM; buparlisib at 1000 nM, singly or in combination, in the presence or absence of exosome inhibitor GW4869 (7.8 ng/mL), for 36 hours. All drug stocks were reconstituted in DMSO and further diluted in media used for cell lines in vitro. DMXAA (positive control) was used as described previously. Supernatants were collected at the end of 36 hours and processed as per ELISA protocol for detection of Type I Interferon, as described above.

Ex Vivo Co-culture Studies with BMDMs. For DNAse I studies. Myc-CAP cells were treated with PARPi or PI3Ki, singly or in combination for 36 hours, and supernatants were treated with 50 units of DNase I in 1× reaction buffer with MgCl₂, and incubated at 37° C. for 30 min. For neutralization of the DNase I reaction, 1 μL of 50 mM EDTA was added to the mix and then incubated at 65° C. for 10 min. Following this step, supernatants −/+ DNase I were added to BMDMs for 30 hours, and IFNβ1 secretion was assessed as described above. To rule out cGAMP as the mediator of STING pathway activation within BMDMs, 10 μg of cGAMP disodium salt (MedChem Express®) was reconstituted in RNA/DNAse free water and pre-treated with 10 units of DNase I. For BMDM STING validation studies, STING^{+/+}/STING^{-/-} BMDMs were co-cultured for 36 hours with supernatants from Myc-CAP and B6-Myc ancer cells that were treated with the indicated drug(s) for 36 hours. Supernatants were collected at the end of treatment and analyzed for IFNβ1 by ELISA. For MV reconstitution studies, Myc-CAP (STING^{lo}) cells were treated with rucaparib (0.5 μM) for 36 hours, followed by isolation of MVs from supernatant (R-MVs), which were then co-cultured with BMDMs in the presence/absence of buparlisib (1 μM) for 36 hours. Cellular metabolites and proteins extracted fro co-cultured BMDMs were used for cGAMP ELISA and for assessment of activation of STING pathway by Western blotting. Supernatants were collected at the end of 30-36 hours and used for detection of Type I IFN-related cytokines and chemokines by cytokine array.

cGAMP assay. For the colorimetry-based detection of cGAMP production in M2 macrophages, cells were treated with MVs isolated from rucaparib-treated Myc-CAP cancer cells, in the presence or absence of buparlisib. Following 30 hours of treatment, the cells were harvested and cell lysates processed using recommended buffers (cGAMP detection kit, Cayman Chemical®) and then used for incubation with anti-cGAMP antibody and detection conjugate for 2 hours or overnight at 4° C. Next, substrate was added and cGAMP was detected at the indicated wavelength, as per manufacturer's instructions.

Quantitative Reverse Transcriptase-Polymerase Chain Reaction (qRT-PCR) for Cytokine/Transcription factor. Snap frozen tumor chunks from in vivo treatment groups were used for isolation of RNA using Qiagen RNeasy Plus isolation kit (Qiagen®), and then used for RT-mediated cDNA synthesis (cDNA RT kit, Bio-Rad®), following which PCR was performed with primers specific for I1-12b and β-actin, using SyBr Green Universal master mix (Bio-Rad®). Each murine sample was analyzed in triplicate on ViiA™ 7 Real-Time PCR System (Applied Biosystems®). Data generated were normalized to β-actin.

Intracellular Staining for Arginase I. Single cells isolated from murine tumor-differentiated BMDMs were processed using BD Cytofix/Cytoperm® solution kit (Fisher Scientific®), as per specified protocol. Anti-mouse Arginase I (R&D Systems®) was used at recommended dilution for staining of permeabilized cells for 40 min at room temperature. For flow cytometry, cells were washed twice with FACS buffer by centrifuging at 500×g for 5 minutes at 4° C. and resuspended in 300 μL of FACS buffer.

Statistical Analysis. One-way ANOVA/Mann Whitney/Unpaired t-test/Paired t-test as well as Kolmogrov Smirnov tests were used for used for statistical evaluation of experimental datasets. The specific statistical tests used for individual experiments are specifically indicated in the descriptions of the drawings.

### Results

The Sparse Immune Infiltrates in Human mCRPC and Murine myc-Driven PC Models are Dominated by Myeloid Suppressive Cells, Particularly Tumor-Associated Macrophages (TAMs).

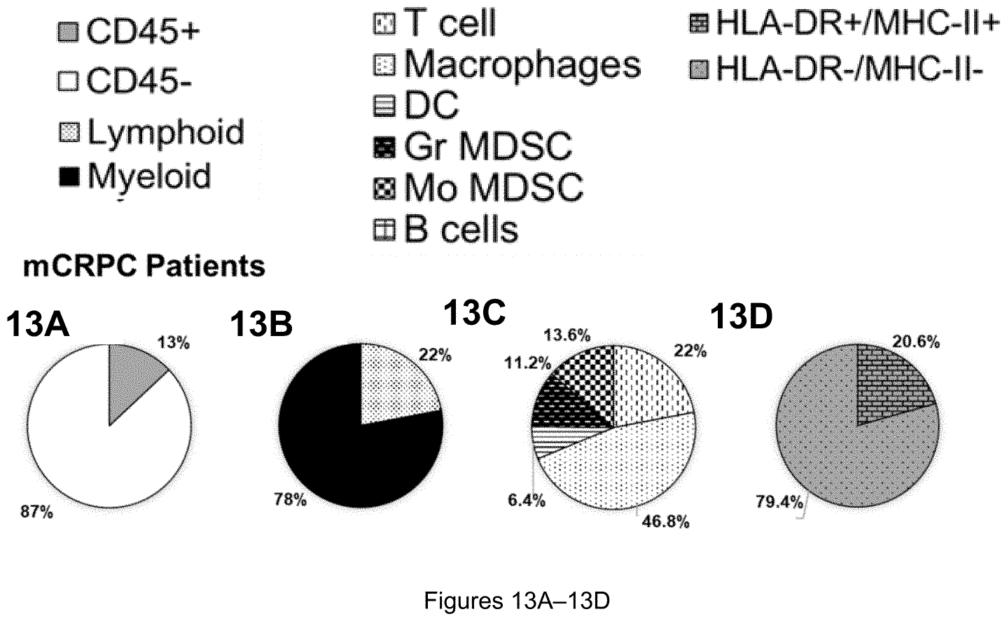

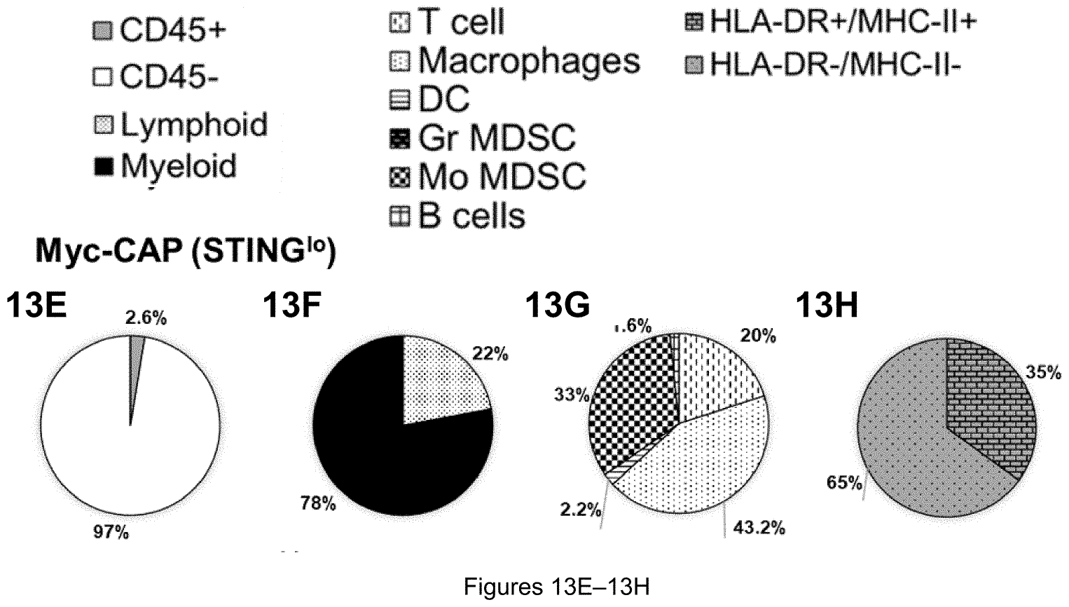

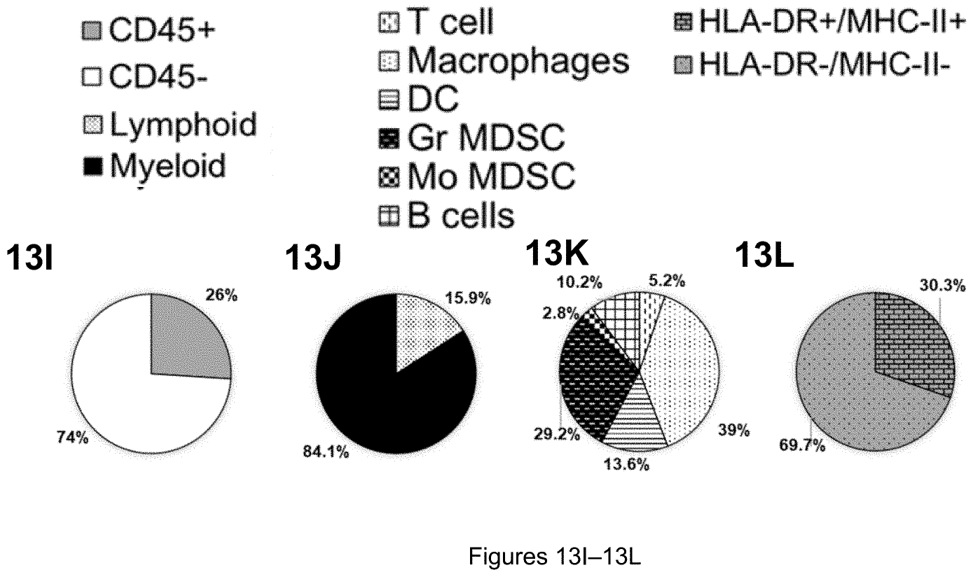

As a first step towards deconvoluting the complex ecosystem of the metastatic tumor immune microenvironment in mCRPC, flow cytometric analysis of 4 tumors isolated from human mCRPC lymph node biopsy samples was performed. Notably, immune profiling revealed an “immune desert” with a paucity of CD45+ cells within the TME (FIG. 13A, Table 1).

TABLE 1
Representative flow cytometric gating strategy, mean and standard deviations for specific immune
subsets in human and murine samples described in FIG. 13. Single cell suspensions from human mCRPC
biopsies, murine Myc-CAP and B6-Myc syngeneic tumors were stained with anti-human/mouse lineage-
specific antibodies and analyzed by flow cytometry. n = 4 for human and n = 3-4 for murine
samples. DC = dendritic cells; MDSC = myeloid derived suppressor cell, h = human, m = mouse.

| | Gating strategy | Average (%) ± SD (%) |
|---|---|---|
| mCRPC patients | | |
| Immune cells | Live+ CD45+ | 13 ± 1.43 |
| Non -Immune cells | Live+ CD45− | 87 ± 1.4 |
| Lymphoid | Live+ CD45+ CD3/CD45+ CD19+ | 22 ± 12 |
| Myeloid | Live+ CD45+ CD11b+ CD3− CD19− | 78 ± 8 |
| T cells | Live+ CD45+ CD3+ | 22 ± 12 |
| B cells | Live+ CD45+ CD3− CD19+ | 0 ± 0 |
| DCs | Live+ CD45+ CD11b+ CD11c+ | 6.4 ± 12 |
| Gr-MDSC | Live+ CD45+ CD11b+ HLA-DR− CD15^{hi}CD33^{lo} | 11.2 ± 3.7 |
| Mo-MDSC | Live+ CD45+ CD11b+ HLA-DR− CD15^{lo}CD33^{hi} | 13.6 ± 7.2 |
| Macrophage | Live+ CD45+ CD11b+ CD163+ CD88+ | 46.8 ± 9.4 |
| Activated Macrophages | Live+ CD45+ CD11b+ CD163+ CD68+ HLA-DR+ | 20.6 ± 19.6 |
| Un-activated Macrophages | Live+ CD45+ CD11b+ CD163+ CD68+ HLA-DR− | 79.4 ± 12.9 |
| Myc-CAP (STING^{lo}) | | |
| Immune cells | Live+ CD45+ | 2.6 ± 0.9 |
| Non -Immune cells | Live+ CD45− | 97.4 ± 0.9 |
| Lymphoid | Live+ CD45+ CD3+/CD45+ CD19+ | 22 ± 4 |
| Myeloid | Live+ CD45+ CD11b+ CD3− CD19− | 78 ± 4 |
| T cells | Live+ CD45+ TCRb+ | 20 ± 8 |
| B cells | Live+ CD45+ TCRb− CD19+ | 1.6 ± 0.9 |
| DCs | Live+ CD45+ CD11b+ CD11c+ | 2.2 ± 1.5 |
| Gr-MDSC | Live+ CD45+ CD11b+ MHC-II− Ly6G^{hi} Ly6C^{lo} | 0 ± 0 |
| Mo-MDSC | Live+ CD45+ CD11b+ MHC-II− Ly6G^{lo} Ly6C^{hi} | 33 ± 6.1 |
| Macrophage | Live+ CD45+ CD11b+ F480+ | 43.2 ± 7.2 |
| Activation state of Macrophages | Live+ CD45+ CD11b+ F480+ MHC-II+ | 35 ± 3 |
| Un-activated Macrophages | Live+ CD45+ CD11b+ F480+ MHC-II− | 65 ± 3 |
| B6-Myc (STING^{hi}) | | |
| Immune cells | Live+ CD45+ | 26 ± 5.8 |
| Non -Immune cells | Live+ CD45− | 74 ± 5.8 |
| Lymphoid | Live+ CD45+ CD3+/CD45+ CD19+ | 15.9 ± 2 |
| Myeloid | Live+ CD45+ CD11b+ CD3− CD19− | 84.1 ± 2.9 |
| T cells | Live+ CD45+ TCRb+ | 5.2 ± 2 |
| B cells | Live+ CD45+ TCRb− CD19+ | 10.2 ± 2 |
| DCs | Live+ CD45+ CD11b+ CD11c+ | 13.6 ± 3.5 |
| Gr-MDSC | Live+ CD45+ CD11b+ MHC-II− Ly6G^{hi} Ly6C^{lo} | 29.2 ± 4 |
| Mo-MDSC | Live+ CD45+ CD11b+ MHC-II− Ly6G^{lo} Ly6C^{hi} | 2.8 ± 1.1 |
| Macrophage | Live+ CD45+ CD11b+ F480+ | 39 ± 3 |
| Activation state of Macrophages | Live+ CD45+ CD11b+ F480+ MHC-II+ | 30.3 ± 3 |
| Un-activated Macrophages | Live+ CD45+ CD11b+ F480+ MHC-II− | 69.7 ± 3 |

Immune profiling is further shown in FIG. 13. Furthermore, the small fraction of immune cells within the TME were predominantly composed of CD11b+ myeloid cells (approx. 80%), with F4/80+ TAMs comprising the highest frequency of CD45+ cells. Approximately 80% of the F4/80+ TAMs within the mCRPC samples were HLADR⁻/MHC II⁻, indicating that these cells are unactivated/immunosuppressive M2-like macrophages. Additionally, immune profiling of tumors derived from two transgenic c-myc-driven prostate cancer lines, Myc-CAP and B6-Myc, generated in FVB/NJ and C57BI/6J genetic backgrounds, respectively, was performed. A similar paucity of CD45+ immune cells within the TME was observed, which was approximately 10-fold lower (p<0.001) in Myc-CAP, relative to the B6-Myc tumors. However, similar to mCRPC samples, both Myc-CAP and B6-Myc tumors showed a relative predominance (approximately 80% of CD45+ immune cells) of CD11b+ myeloid cells within the TME. Within the CD11b+ myeloid population, F4/80+ TAMs comprise the predominant immune subset, and 65-70% of these cells were unactivated/immunosuppressive (M2) HLADR⁻/MHC II⁻ macrophages, similar to what was observed in the mCRPC patient samples. Taken together, these data demonstrate similar immune contexture in human mCRPC and murine c-myc-driven cancers, which is dominated by myeloid suppressive cells, particularly TAMs.

Expression of STING and an Activated Myeloid Gene Expression within Primary PC Samples Positively Correlate with Biochemical Recurrence-Free Survival.

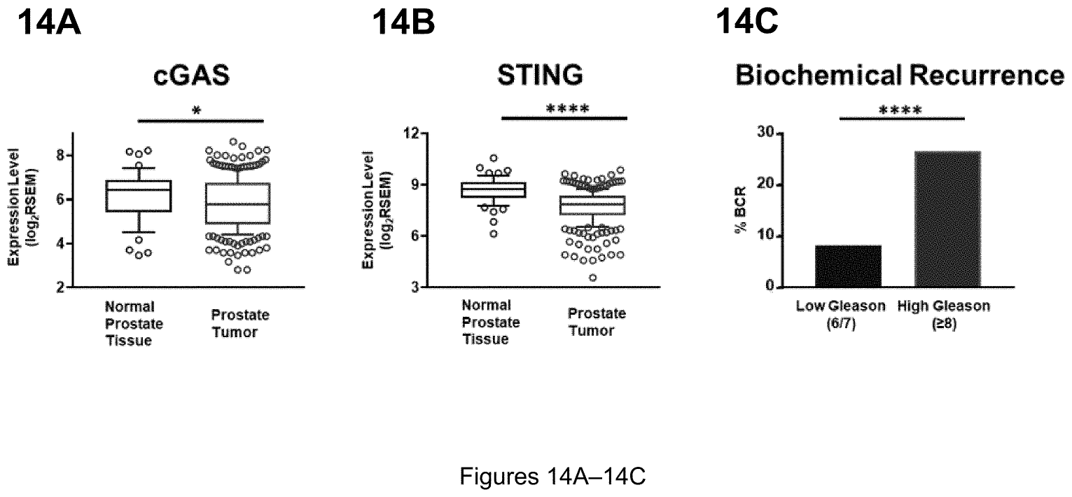

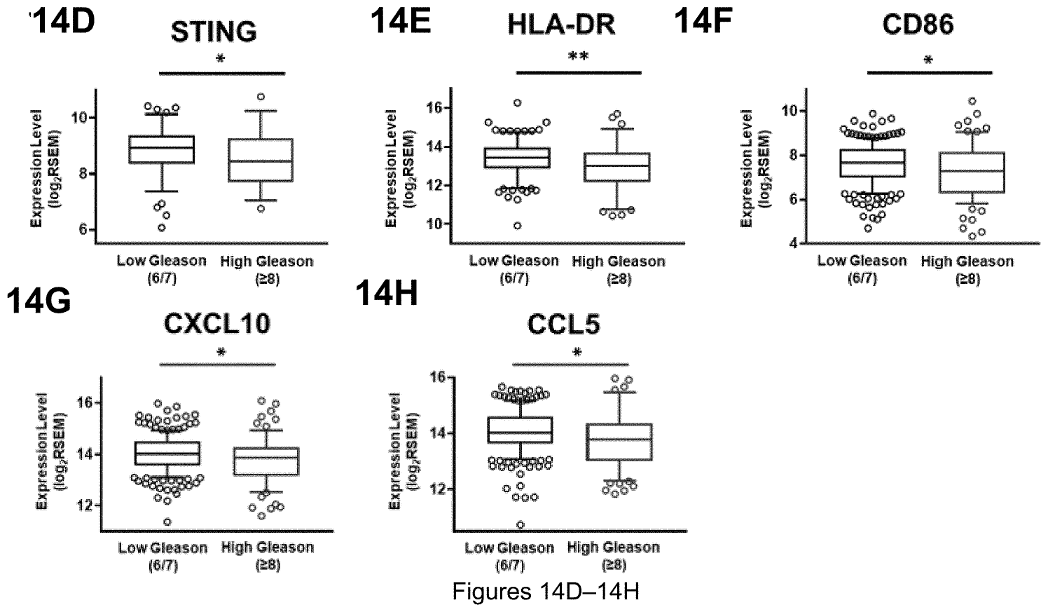

Next, cGAS and STING expression was assessed in primary human PC samples within the TCGA, and confirmed to have reduced expression relative to normal tissue counterparts (FIGS. 14A-14B). Furthermore, transcriptomic data analysis of primary PC samples within the TCGA revealed that high risk (Gleason≥8) patients with higher biochemical recurrence (FIG. 14C) had decreased gene expression of STING (FIG. 14D), myeloid activation markers HLA-DR and CD86, (FIGS. 14E-14F), and T cell chemotactic factors CCLS and CXCL10 expression (FIGS. 14G-14H), relative to low-risk (Gleason 6/7) patients. These data identify a positive correlation between STING expression, myeloid activation states, T cell chemotactic factor expression, and clinical outcome.

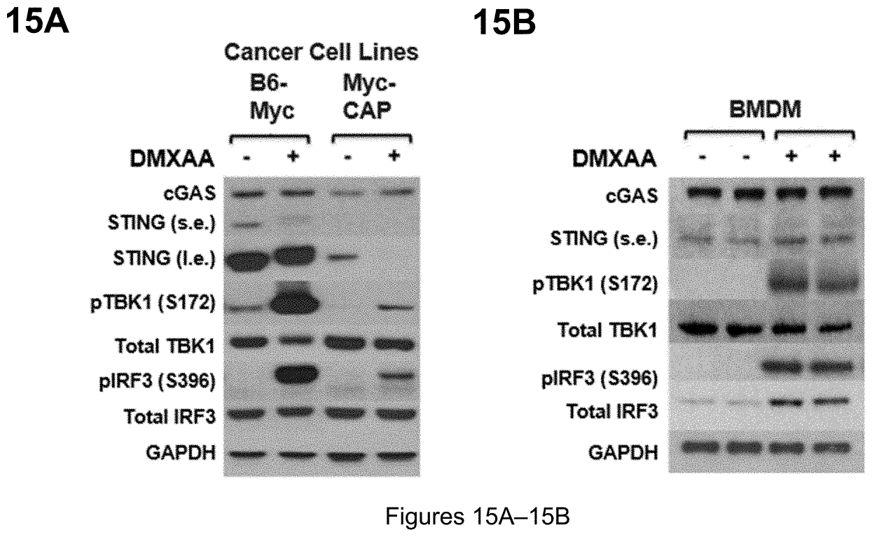

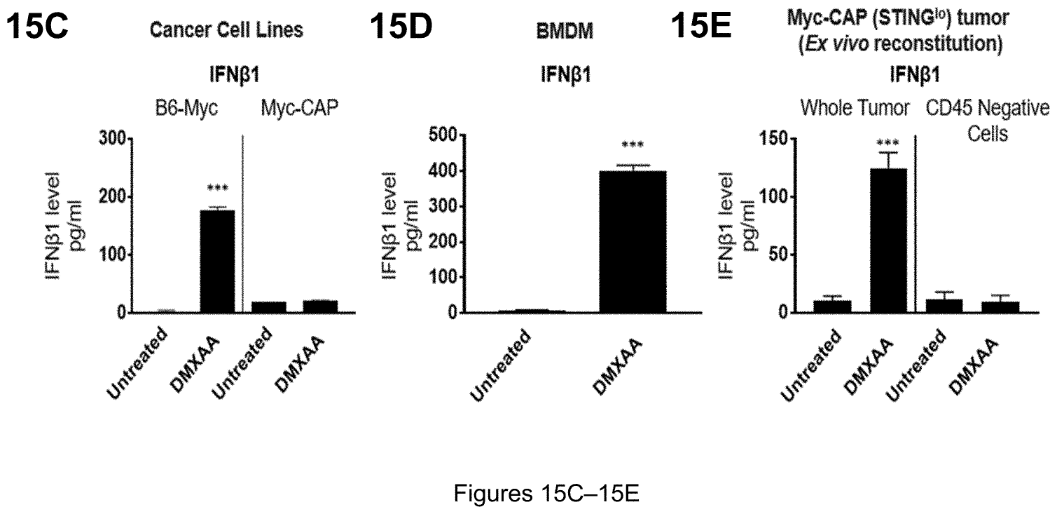

Next, cGAS and STING protein expression in Myc-CAP and B6-Myc cells was assessed, and low and high cGAS/STING expression in Myc-CAP (STING^{lo} and B6-Myc (STING^{hi}) cells, respectively (FIG. 15A), was observed, which mimics the STING gene expression patterns observed in low and high-risk PC subgroups within the TCGA (FIG. 14). Consistent with the relative expression data of STING pathway components in B6-Myc (STING^{hi}) cancer cells, it was observed that deliberate STING activation with DMXAA (mouse STING agonist) activated the cGAS/STING signaling pathway components phospho-TANK-binding kinase 1 (p-TBK1) and phospho-interferon regulatory factor-3 (p-IRF3) in B6-Myc (STING^{hi}) cells (FIG. 15A), but was not observed in Myc-CAP (STING^{lo}) cells. DMXAA treatment of bone marrow derived macrophages (BMDMs) also activated the c-GAS/STING signaling pathway (FIG. 15B). Furthermore, ELISA analysis revealed that DMXAA treatment of B6-Myc (STINGhi) cancer cells and BMDMs elicited a 48.1-fold and 46.4 fold induction in IFN-β levels, not observed in Myc-CAP (STING^{lo}) cancer cells (FIGS. 15C, 15D). Interestingly, ex vivo treatment of single cell suspensions derived from Myc-CAP (STING^{lo}) tumors with DMXAA demonstrated a 11.4-fold increase in IFN-β levels within the TME, but a lack of response specifically within the CD45-negative tumor cell fraction (FIG. 15E). Collectively, these data demonstrate that STING pathway can be activated in a tumor cell extrinsic manner within Myc-CAP (STING^{lo}) tumors. On the other hand, B6-Myc (STING) tumors can turn on the STING pathway in both tumor cell intrinsic and extrinsic compartments.

### Concomitant PI3Ki Sensitizes B6-Myc (STING^{hi}) Murine PC to PARPi/aPD-L1 Combination Therapy, via STING-Dependent, TAM-Driven Immune Mechanism.

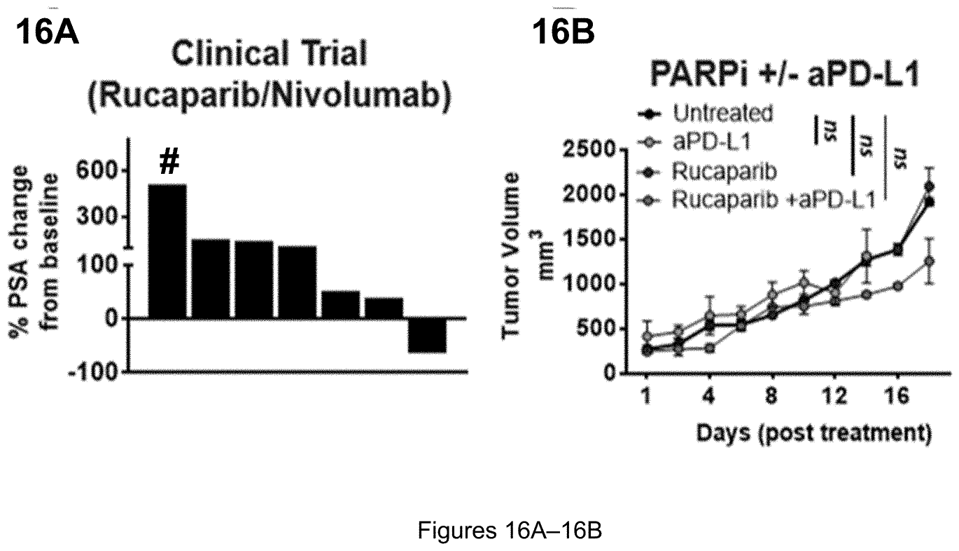

Recent preclinical data has demonstrated that PARP inhibitor (PARPi)-induced DNA damage can reprogram the tumor immune microenvironment via tumor cell intrinsic cGAS/STING activation, thereby enhancing T cell infiltration and efficacy of ICB in homologous recombination deficient (HRD) breast and ovarian cancer models. Given preclinical data suggesting that PARPi can induce DNA damage in HR-proficient cancers, it was hypothesized that PARPi-induced DNA damage can enhance ICB efficacy, independent of HR status, in mCRPC patients enrolled in an investigator-initiated co-clinical trial at the University of Chicago (NCT03572478, IRB18-0154). To test this hypothesis, mCRPC patients that had progressed on at least one-line of AR-targeted therapy in the castrate-resistant setting, were treated with PARPi rucaparib (Clovis Oncology®) in combination with nivolumab (Bristol Myers Squibb®) until disease progression and/or unacceptable toxicity. A Waterfall plot for mCRPC patients who were on the study for at least 90 days demonstrated that only 1 of 7 evaluable patients responded to the combination therapy (FIG. 16A). The single responder patient harbored a BRCA2 mutation that was predicted to respond to PARPi monotherapy, while the remaining patients had an HRD-proficient tumor mutational status. The combination of rucaparib and nivolumab had a PSA response rate of 9% (1/11 patients) and an objective response rate, per RECIST/PCWG3 criteria, of 0% (0/11 patients). Median progression-free survival for mCRPC patients on the trial was 2.96 months (95% Confidence Interval, 2.03 months-not assessable). Taken together, these data demonstrate that the majority of patients did not exhibit clinically meaningful responses to rucaparib/nivolumab combination therapy. Consistent with clinical trial data, de novo resistance of B6-Myc (STING^{hi}) syngeneic tumors to rucaparib or rucaparib/PD-L1 antibody combination was observed (FIG. 16B).

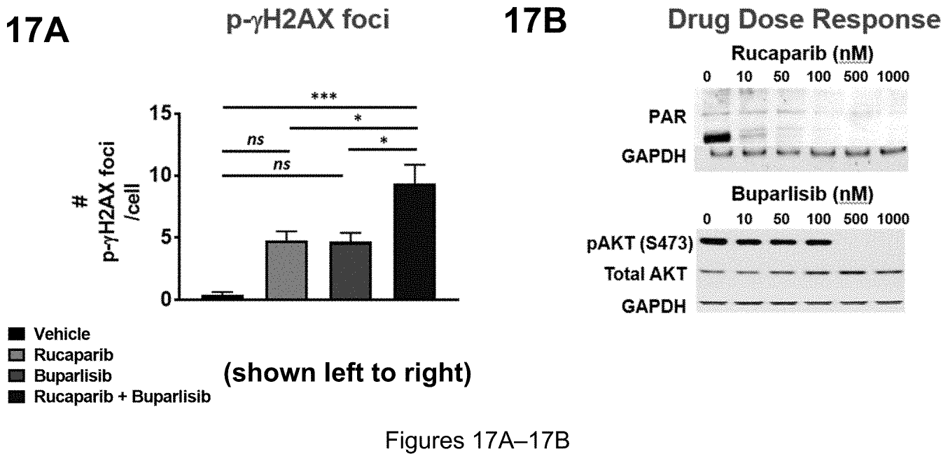

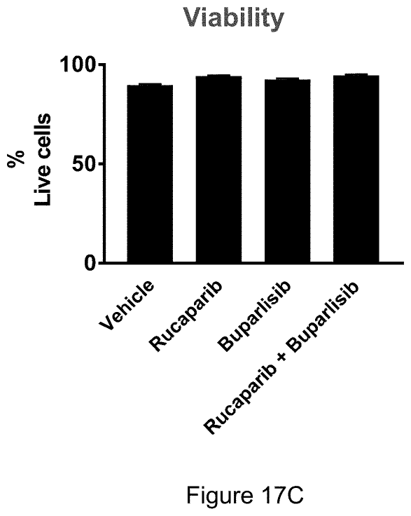

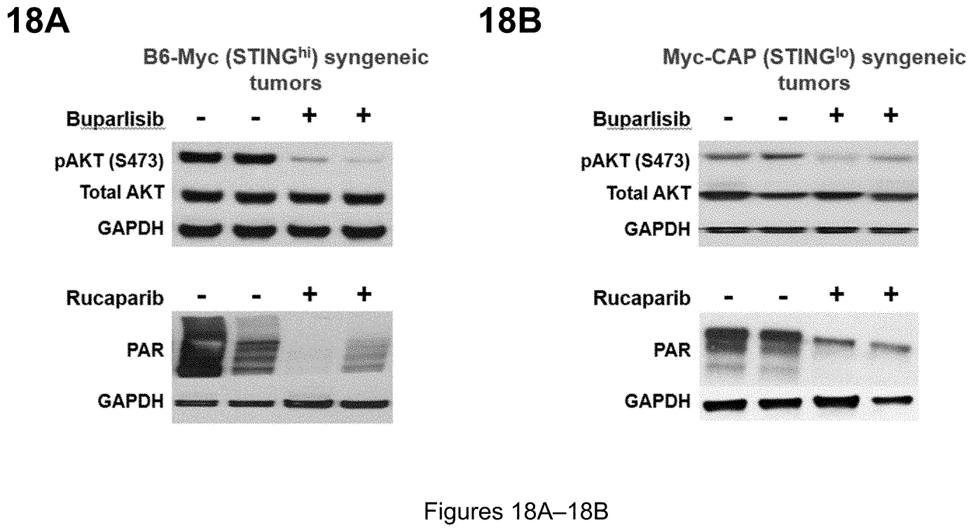

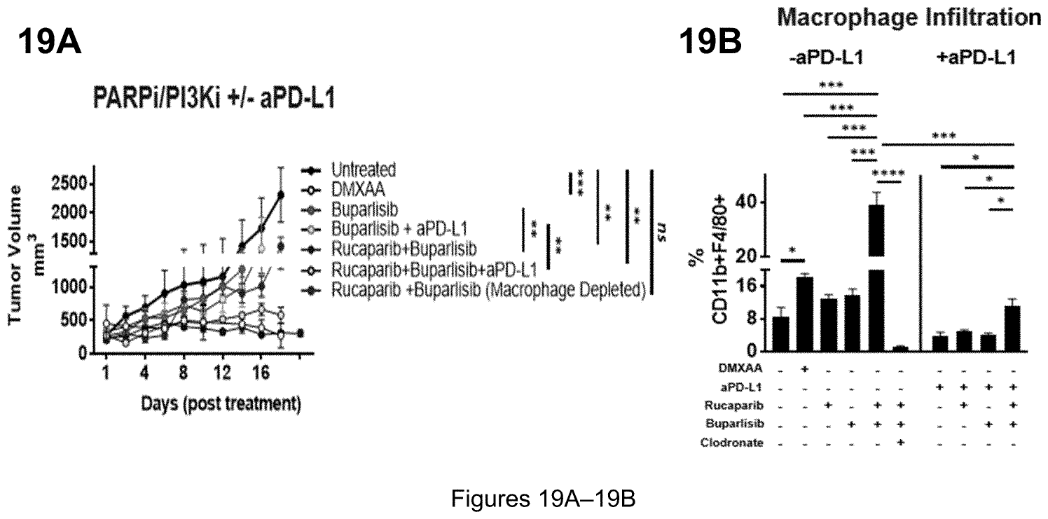

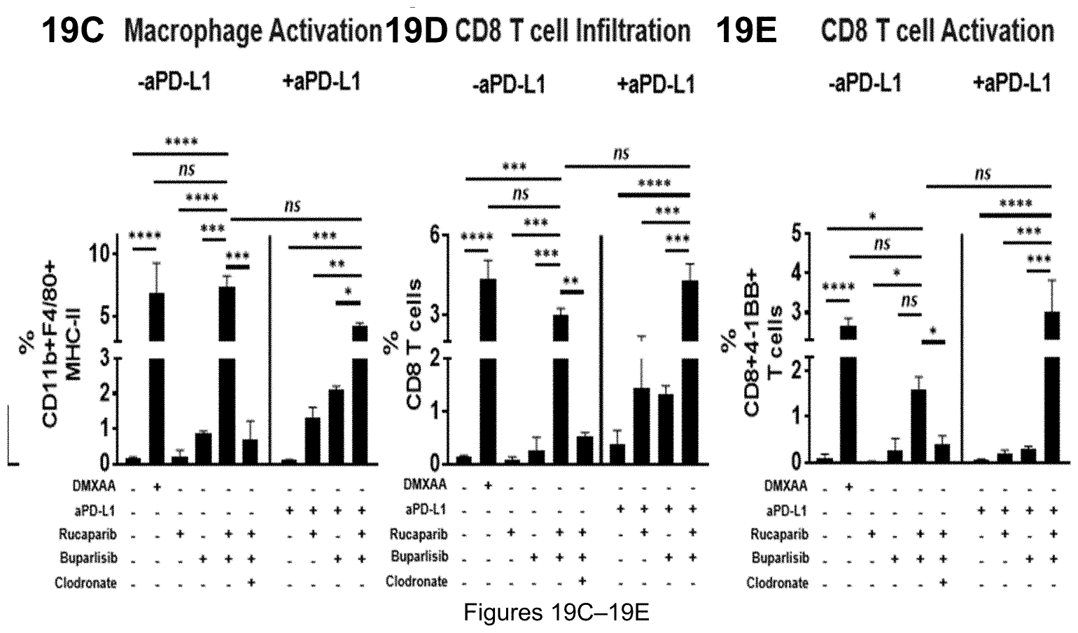

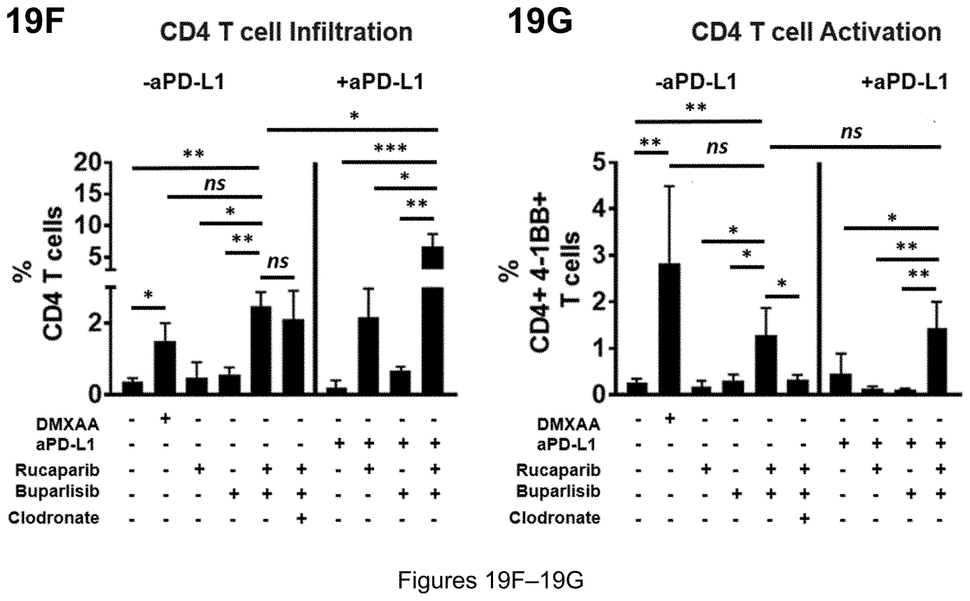

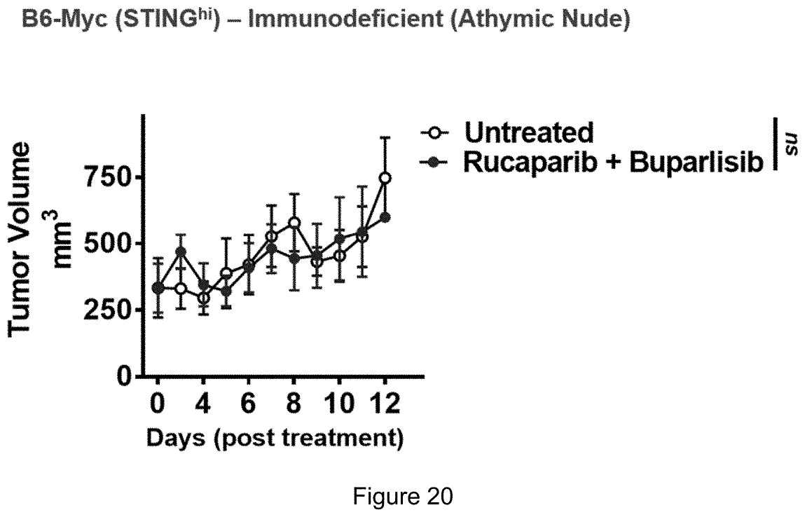

To address the mechanistic basis for why PARPi was insufficient to sensitize B6-Myc (STING^{hi}) tumors, B6-Myc was treated in vitro with rucaparib at 500 nm concentration that completely inhibits PARylation, and its impact on DNA damage was evaluated, as assessed by quantification of p-γH2AX foci (marker of dsDNA breaks) using confocal microscopy. Interestingly, B6-Myc cells did not show a statistically significant increase in DNA double-strand breaks (DSBs) following single-agent rucaparib treatment (FIG. 17A). Several studies have demonstrated that combination of PARP inhibitors (PARPi) and pan-PI3K (PI3Ki) inhibitors induce additive DNA damage and tumor regression in prostate and endometrial cancers (Bian X, Gao J, Luo F, Rui C, Zheng T, Wang D, et al. PTEN deficiency sensitizes endometrioid endometrial cancer to compound PARP-PI3K inhibition but not PARP inhibition as monotherapy. Oncogene 2018; 37(3):341-51; Gonzalez-Billalabeitia E, Seitzer N, Song S J, Song M S, Patnaik A, Liu X S, et al. Vulnerabilities of PTEN-TP53-deficient prostate cancers to compound PARP-PI3K inhibition. Cancer Discov 2014; 4(8):896-904). Consistent with these prior observations, a statistically significant additive increase in DNA DSBs was observed following concomitant treatment with rucaparib and buparlisib (pan-PI3K inhibitor), but not with corresponding single-agent treatments in B6-Myc (STING^{hi}) cells in vitro (FIG. 17A). Using concentrations for rucaparib and buparlisib of 500 nM and 1 μM, respectively, that achieved complete target inhibition in vitro (FIG. 17B), drug combination studies revealed that there was no change in viability of B6-Myc cells (FIG. 17C). Next, the impact of a rucaparib and buparlisib combination was tested, with or without PD-L1 antibody, on the ability to control tumor growth of B6-Myc (STING) syngeneic mice in vivo. At doses that pharmacodynamically inhibit PARP and PI3K enzymatic activity within the tumor in vivo (FIG. 18), complete tumor regression was observed with the rucaparib/buparlisib combination relative to either single-agent, and it was maintained with the addition of anti-PD-L1 antibody. This tumor clearance and immune activation elicited by the rucaparib/buparlisib combination was phenocopied by DMXAA (mouse STING agonist) administration (FIGS. 19A-19G). To address the discordance between in vitro cytotoxicity data and in vivo anti-cancer responses observed with rucaparib/buparlisib combination, it was hypothesized that rucaparib/buparlisib combination is working predominantly via a tumor cell extrinsic immune mechanism. To test this possibility, the impact of the rucaparib/buparlisib combination therapy was evaluated in immunodeficient athymic nude mice implanted with B6-Myc (STING^{hi}) allograft tumors. Strikingly, the anti-cancer mechanism of rucaparib/buparlisib was abolished in immunodeficient athymic nude mice (FIG. 20), thus demonstrating that this combination drives tumor regression via a cancer cell non-autonomous immune mechanism.

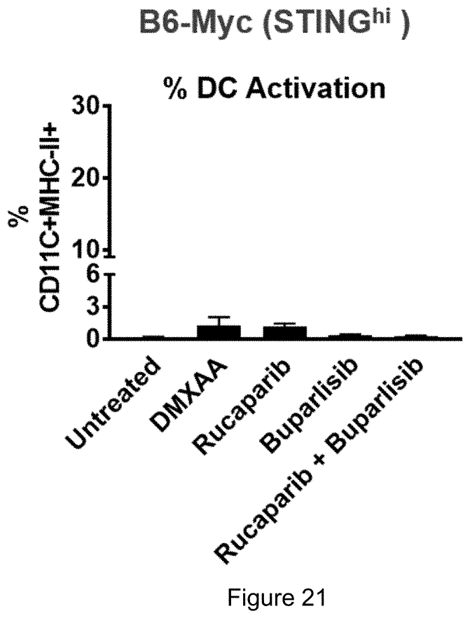

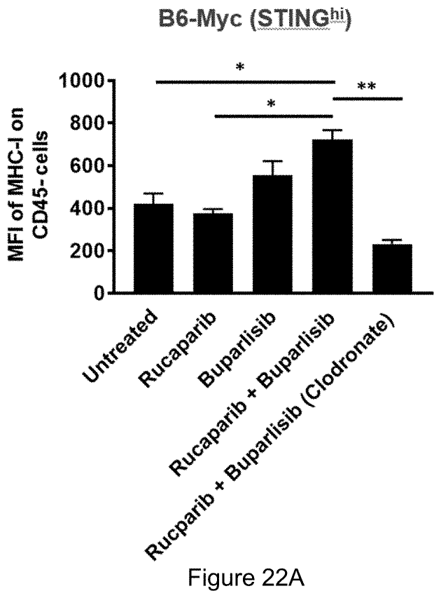

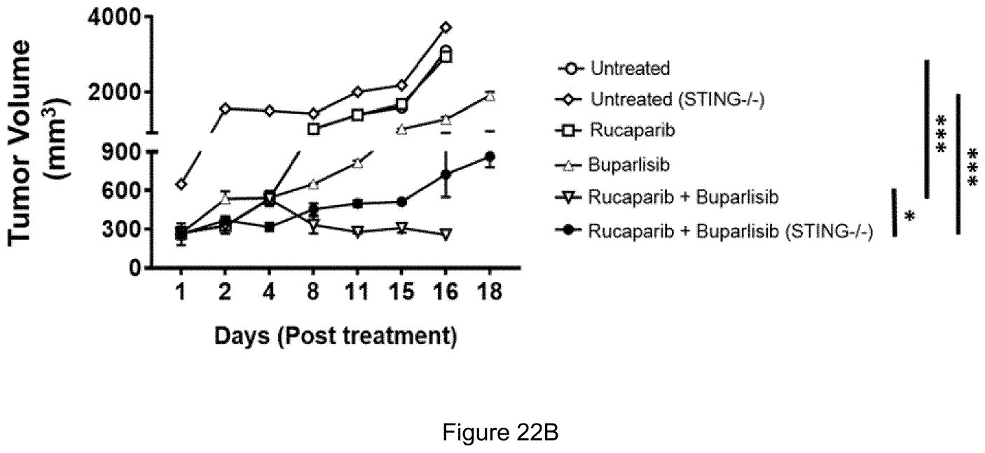

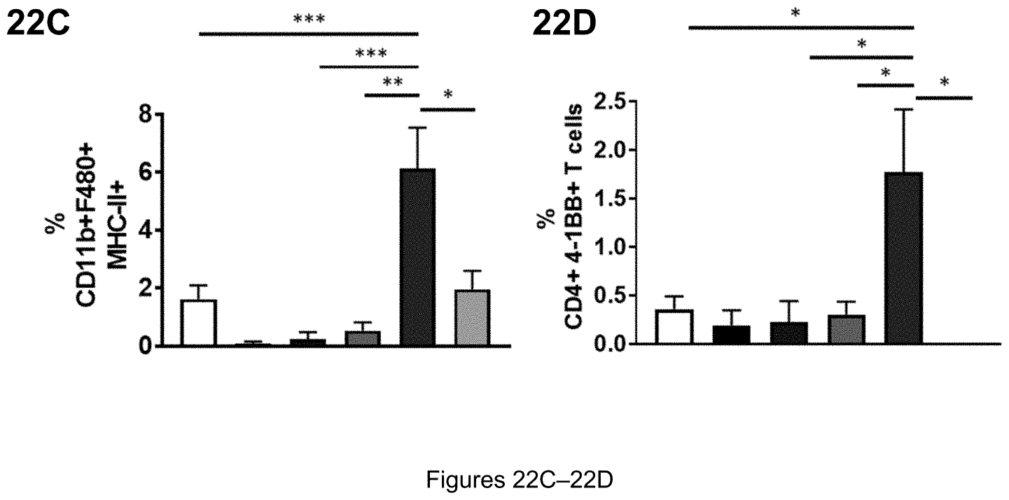

Immune TME profiling studies revealed that rucaparib/buparlisib combination in syngeneic B6-Myc (STING) demonstrated an increase in macrophage infiltration (FIG. 19B) and activation (FIG. 19C) but not dendritic cell (DC) activation (FIG. 21) and was accompanied by an increase in CD4 and CD8 T cell infiltration (FIGS. 19D, 19F) and activation (FIGS. 19E, 19G), respectively, relative to corresponding single-agent controls. Furthermore, the addition of PD-L1 antibody to rucaparib/buparlisib combination accentuated the anti-tumor immune responses, particularly, CD4 infiltration, relative to buparlisib/rucaparib treatment (FIG. 19F). Critically, the immunologic changes within the TME and tumor clearance elicited by PARPi/PI3Ki treatment were significantly attenuated by systemic macrophage depletion with clodronate (FIG. 19), suggesting a macrophage-driven (DC-independent) anti-cancer innate immune mechanism for this combination. Furthermore, the anti-tumor immune response elicited by rucaparib/buparlisib combination increased MHC Class I expression within tumor cells, which was also suppressed by clodronate (FIG. 22A), likely related to suppression of IFN-Γ-producing T cell infiltration/activation following TAM depletion.

To elucidate the role of STING pathway activation and the relative contributions of tumor cell intrinsic vs. extrinsic STING on the observed tumor regression, B6-Myc (STING^{hi}) tumor allografts were implanted into STING^{-/-} C57BI/6J mice. Strikingly, PARPi/PI3Ki-mediated B6-Myc tumor regression was partially attenuated in STING^{-/-} C57BL6 mice, and resulted in reduced macrophage and T cell activation, relative to their STING^{+/+} counterparts (FIGS. 22B-22D). Taken together, these results demonstrate that PARPi/PI3Ki can induce tumor regression in B6-Myc (STING^{hi}) syngeneic model via an innate immune mechanism that is driven by tumor cell extrinsic host STING within TAMs.

PARPi/PI3Ki in Combination with Androgen Deprivation Therapy (ADT) Causes Tumor Regression In Vivo in Myc-CAP (STING^{lo} ) Tumors, which is Driven by TAM-Mediated Anti-Cancer Innate Immunity.

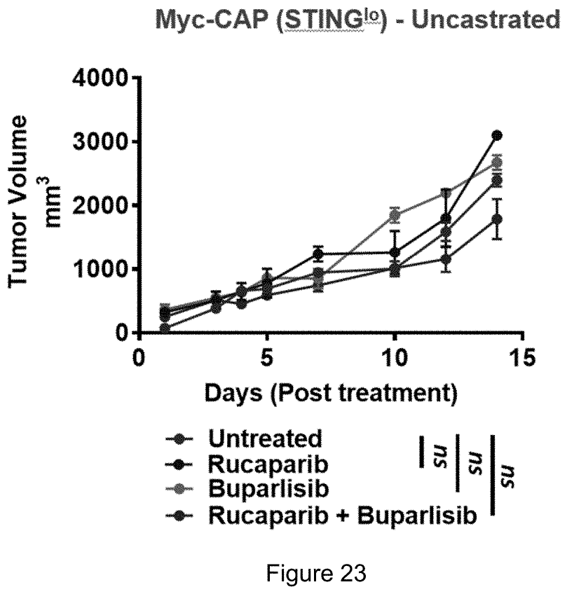

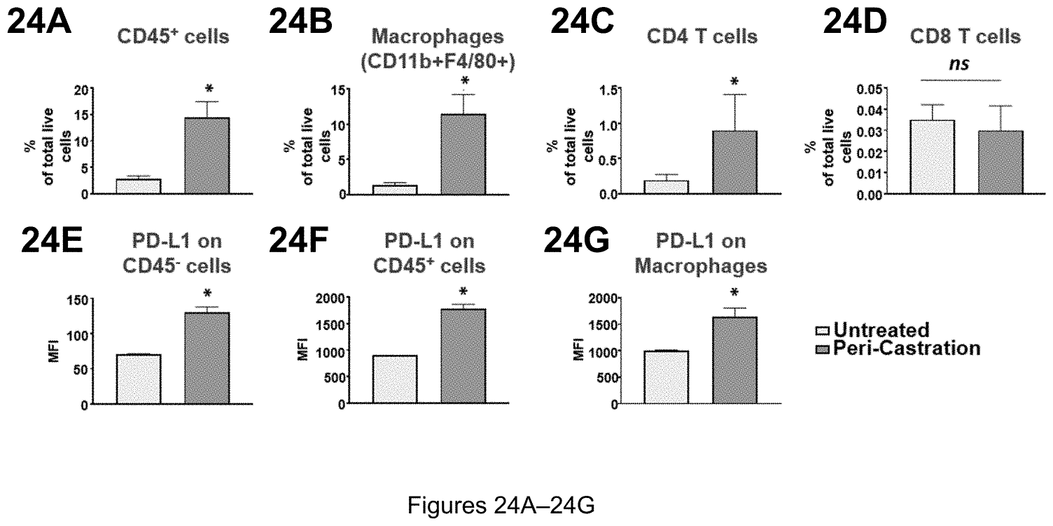

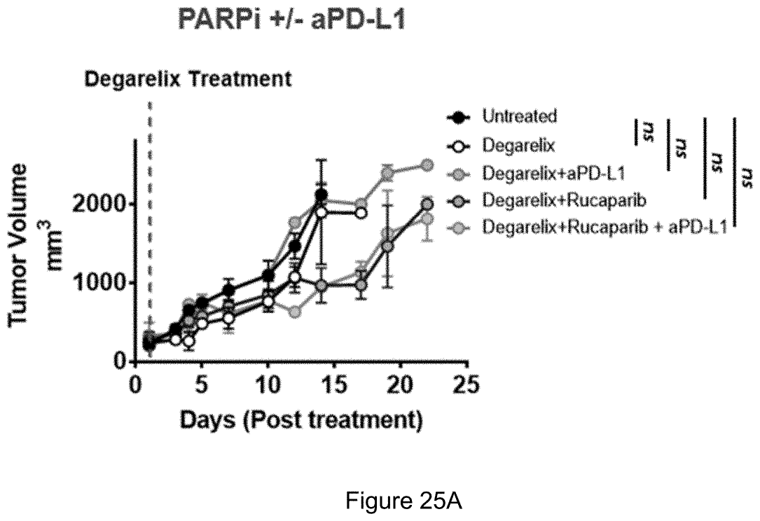

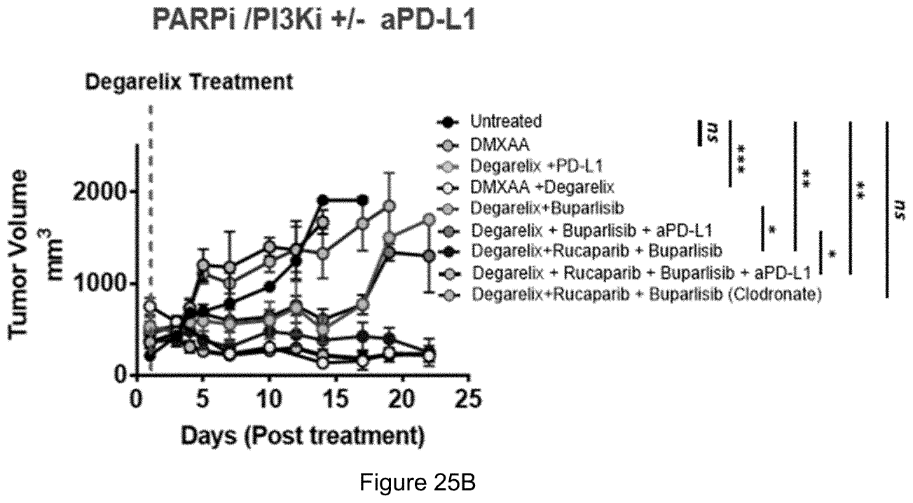

While PARPi/PI3Ki was sufficient to induce tumor clearance in B6-Myc (STING^{hi}) syngeneic mice, Myc-CAP (STING^{lo}) syngeneic mice are de novo resistant to this combination (FIG. 23). This could be related to the growth of these tumors in the FVB/NJ genetic background, which is known to have low immunogenicity. Prior studies have demonstrated that early stages of castration, which is standard-of-care for advanced prostate cancer, induce T-effector and T-regulatory cell infiltration within prostate tumors. Consistent with prior data, an approximately 5-fold increase in CD45+ infiltration in Myc-CAP (STING^{lo}) tumors was observed following castration, predominantly driven by an increase in the TAM subset, with a smaller contribution of CD4+ T cell subsets, but not CD8+ T cells (FIGS. 24A-24D). In addition, there is an increased PD-L1 expression in CD45− fractions and CD45+ (particularly TAMs) within the TME following castration (FIGS. 24E-24G). However, castration alone or in combination with rucaparib and/or PD-L1 antibody was insufficient to control tumor growth in Myc-CAP (STING^{lo}) syngeneic mice (FIG. 25A), which was consistent with recent co-clinical trial data of rucaparib/nivolumab in mCRPC patients (FIG. 16A).

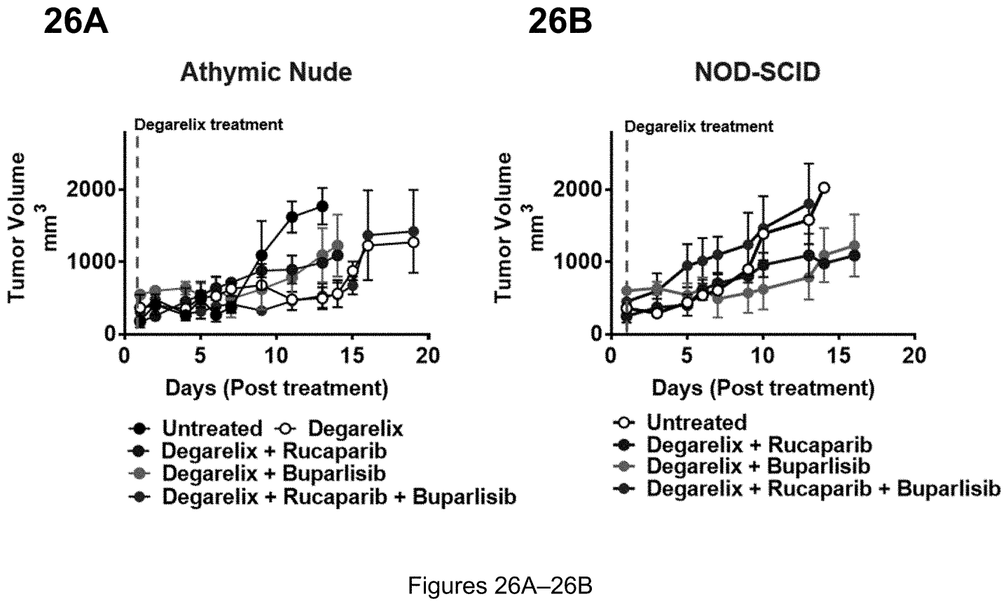

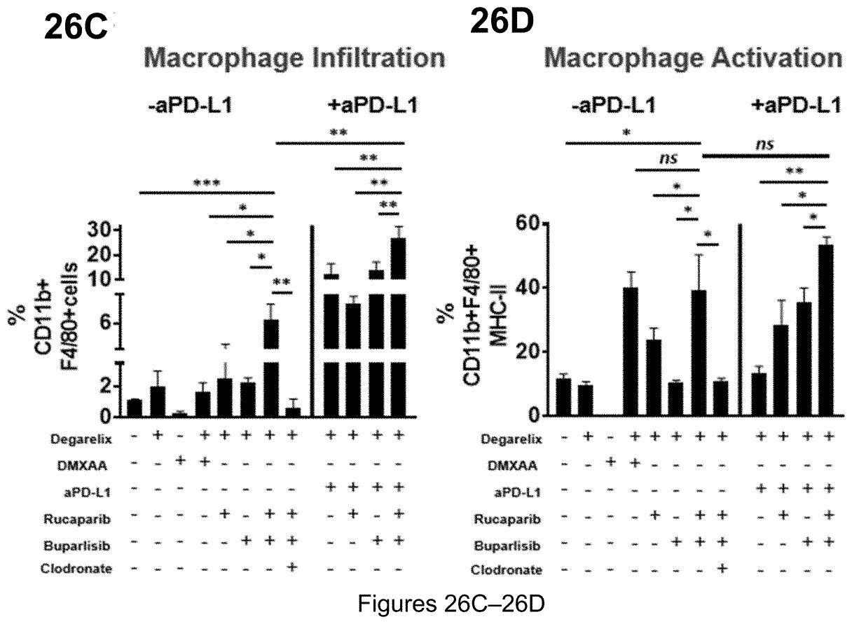

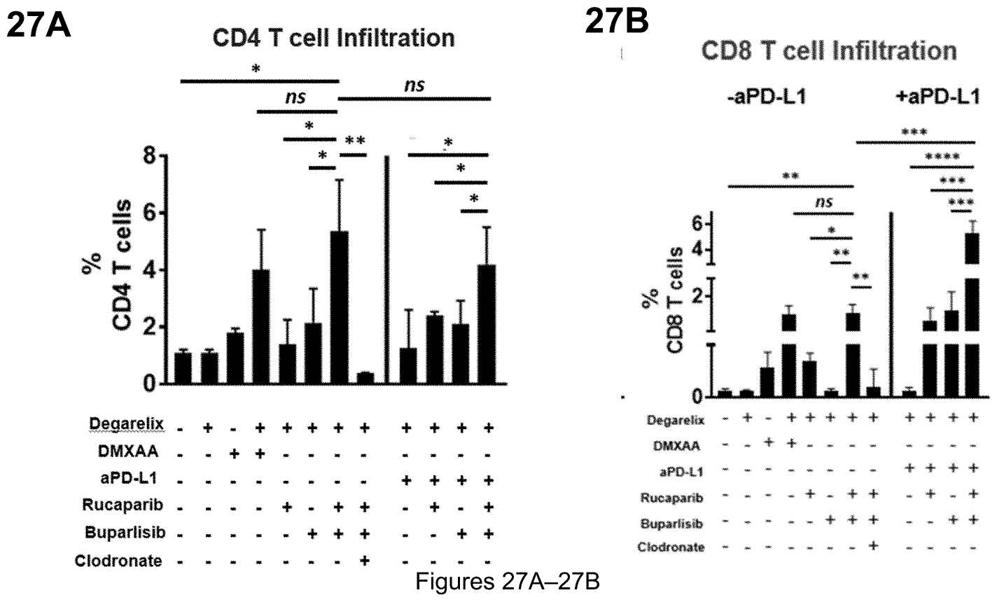

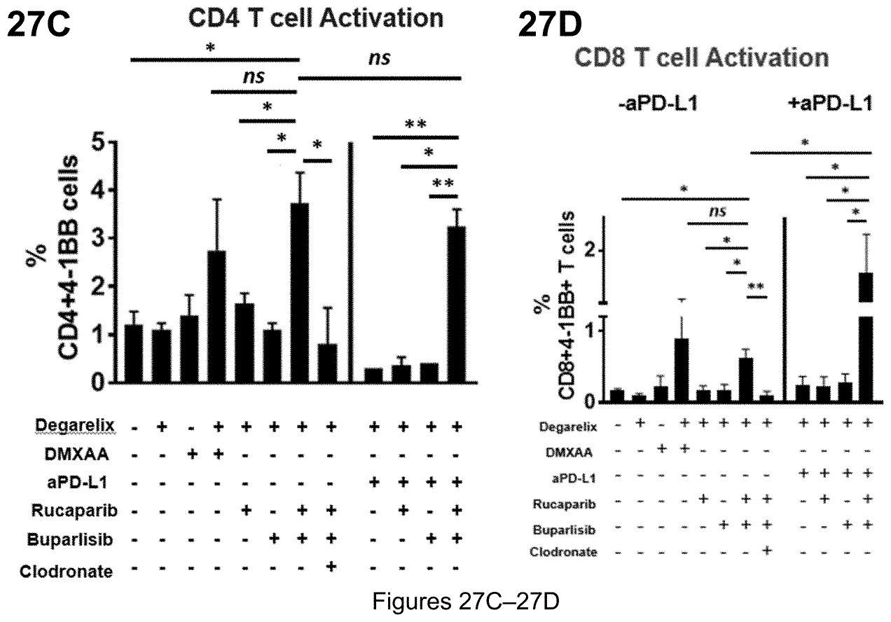

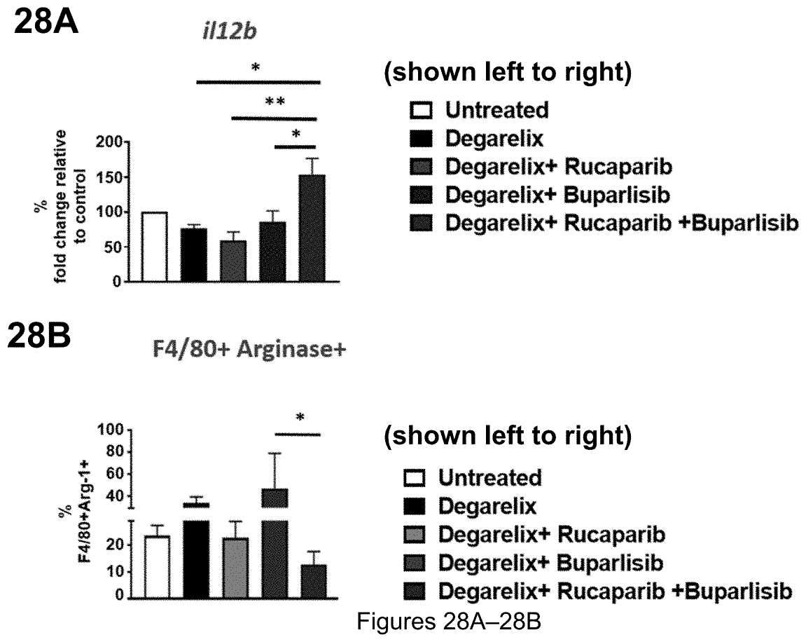

The hypothesis that the immunostimulatory effects of castration would lower the threshold for the PARPi/PI3Ki combination to elicit a potent anti-tumor response in Myc-CAP (STING^{lo}) syngeneic mice was next tested. Strikingly, it was observed that the combination of degarelix and rucaparib/buparlisib resulted in complete tumor regression in Myc-CAP (STING^{lo}) syngeneic mice that was not observed with single agent degarelix, rucaparib, and buparlisib, or their corresponding doublet therapies (FIG. 25B). The combination of degarelix and DMXAA phenocopied the complete tumor clearance observed with degarelix/rucaparib/buparlisib. Importantly, the anti-tumor response observed with degarelix/rucaparib/buparlisib was abolished in immunodeficient athymic nude (FIG. 26A) and NOD/SCID mice (FIG. 26B), thus demonstrating that this regimen induces tumor control via an immune-dependent mechanism. Furthermore, degarelix/rucaparib/buparlisib triple combination led to an increase in macrophage infiltration (FIG. 26C) and activation (FIG. 26D), which was accompanied by an increase in CD4 and CD8 T cell infiltration (FIGS. 27A, 27B) and activation (FIGS. 27C, 27D), respectively, relative to corresponding singlet or doublet controls. Furthermore, this immune activation effect of degarelix/rucaparib/buparlisib was accentuated by PD-L1 antibody, specifically with respect to macrophage infiltration and CD8 infiltration/activation (FIGS. 26C, 27B, 27D). Gene expression (qRT-PCR) analysis and flow cytometric analysis of tumor extracts revealed increased il12b expression (FIG. 28A) and decreased Arginase-1 expression (FIG. 28B), respectively, within TAMS from degarelix/rucaparib/buparlisib-treated tumors relative to corresponding singlet or doublet controls, thus demonstrating that the triple combination enhanced M1 macrophage polarization within the TME. Furthermore, it was observed that concomitant clodronate treatment abolished the tumor regression and immune-permissive reprogramming observed with degarelix/rucaparib/buparlisib treatment, with or without PD-L1 antibody (FIGS. 25B, 26C-26D, 27A-27D), similar to what was observed in the B6-Myc (STING) model. Taken together, these data demonstrate that ADT/PARPi/PI3Ki combination induces a macrophage STING-mediated, innate immune response in Myc-CAP (STING^{lo}) syngeneic model, resulting in tumor clearance. Because the ADT/PARPi/PI3Ki-mediated tumor clearance was abolished in athymic nude mice, the anti-cancer responses were mediated at least in part by macrophage-mediated activation of T cell immunity.

PARPi/PI3Ki-Induced STING Pathway Activation within TAMs is Mediated via MVs Released from Tumor Cells.

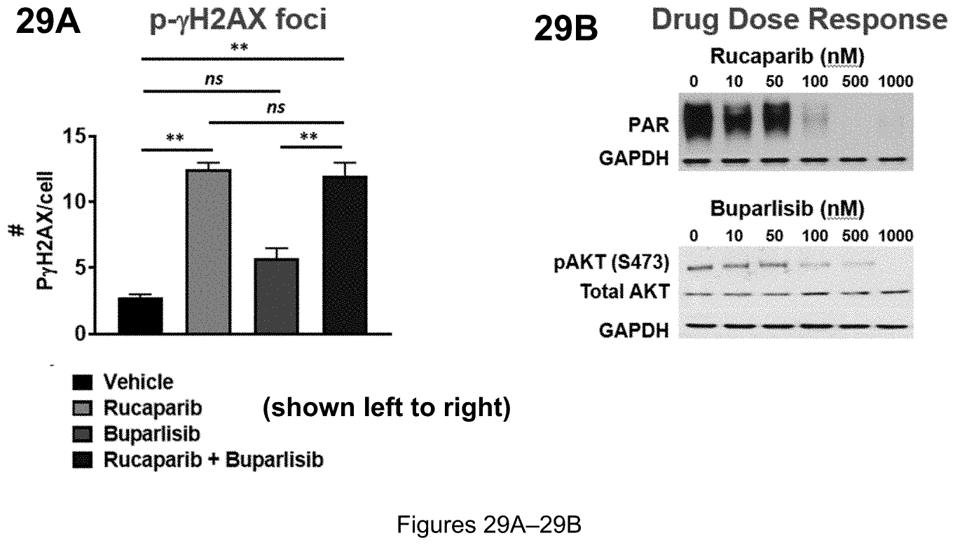

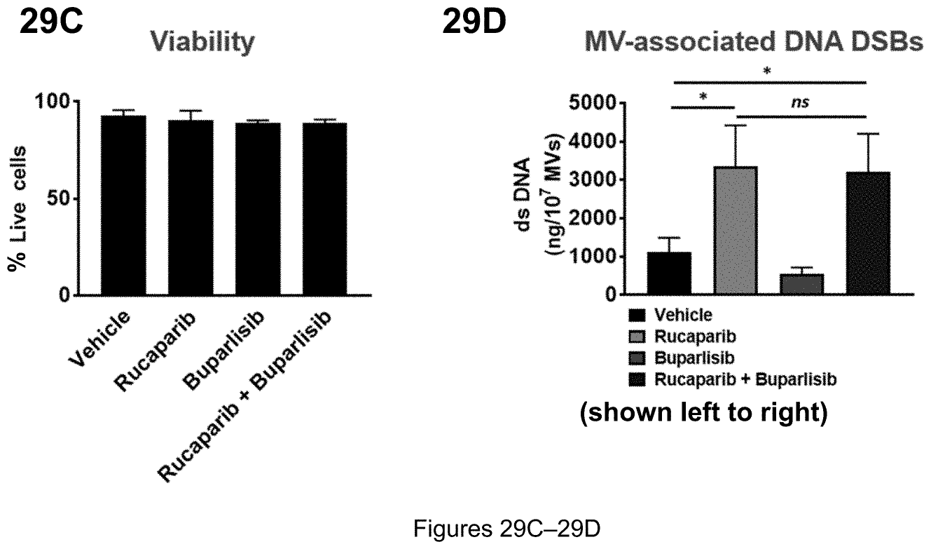

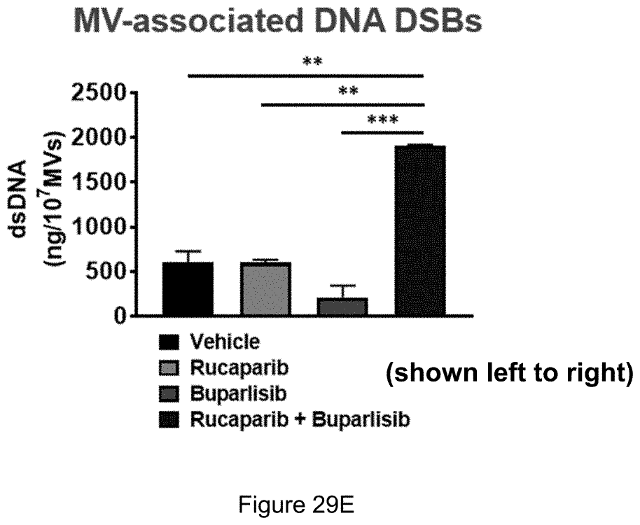

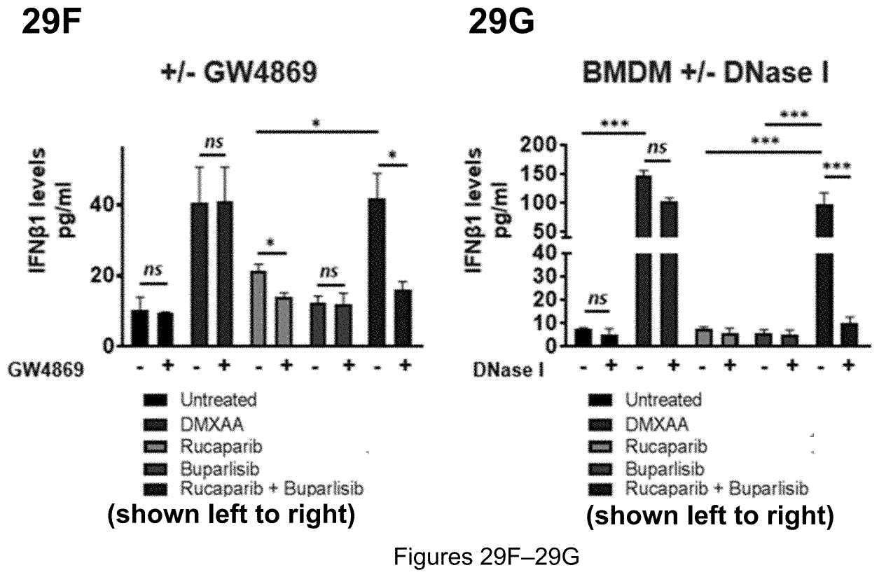

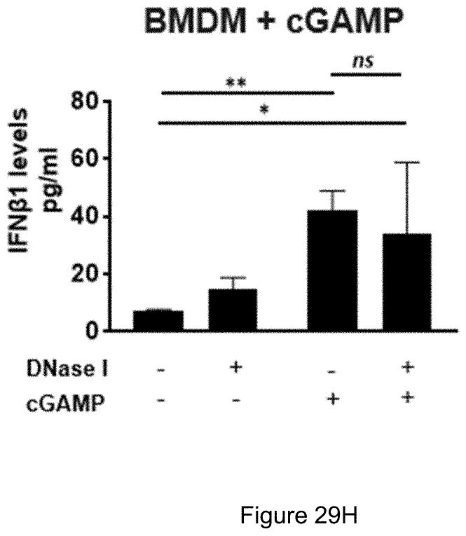

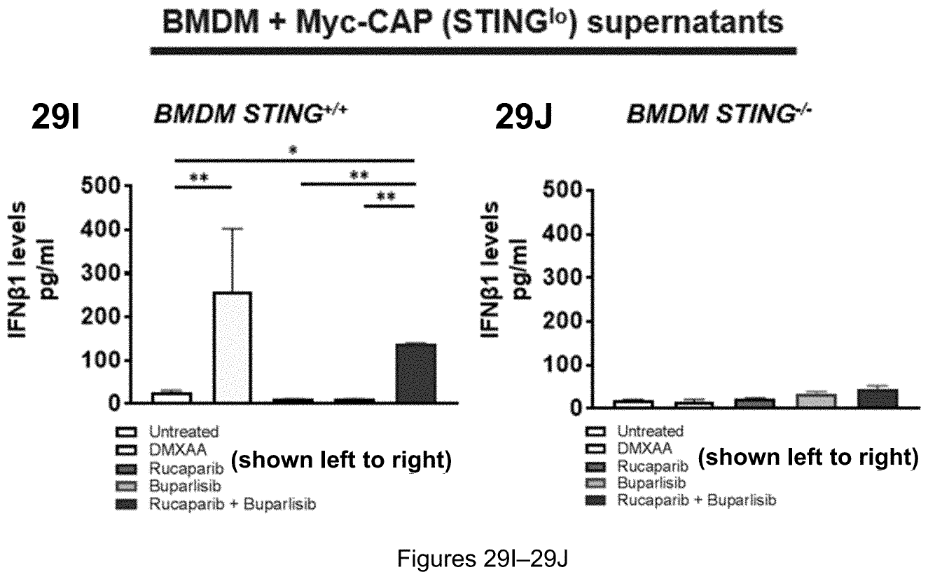

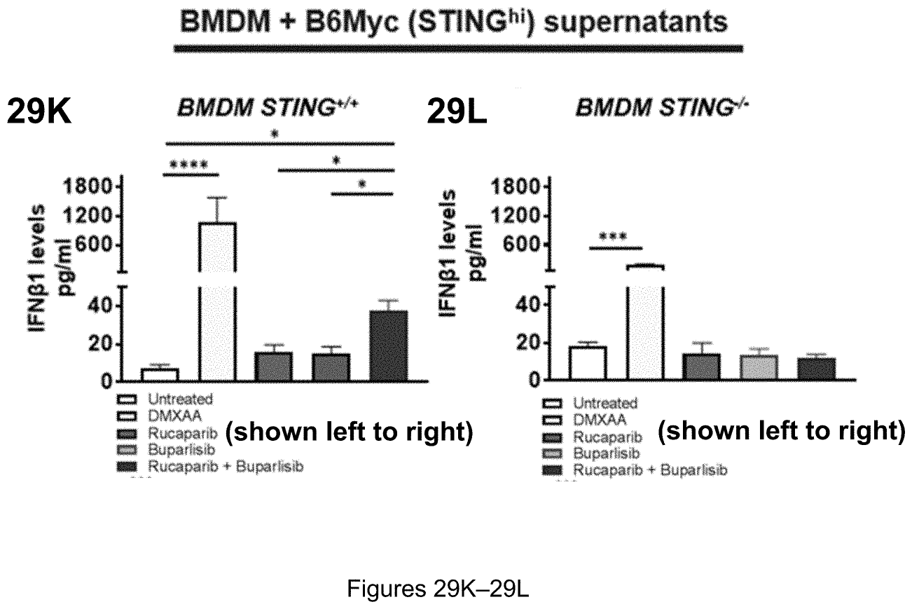

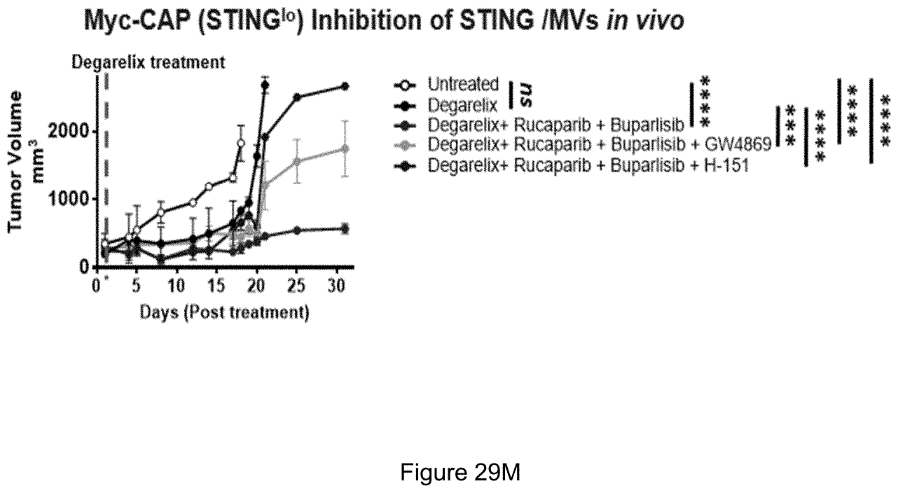

To determine whether PARPi/PI3Ki combination elicits DNA DSBs in Myc-CAP) (STING^{lo}) context, cells were treated in vitro with PARPi rucaparib, singly and in combination with buparlisib, at their respective target inhibitory concentrations (FIGS. 29A-29D). Interestingly, it was observed that treatment of Myc-CAP cells with rucaparib caused an increase in DNA DSBs, as measured by number of p-γH2AX foci, which was not enhanced by the addition of PI3Ki (FIG. 29A). In addition, there was no change in the viability of Myc-CAP cells treated at complete target inhibition concentrations of 500 nM and 1 μM for rucaparib and buparlisib (FIG. 29B), respectively, singly and in combination (FIG. 29C). Given the requirement of dual PARP and PI3K inhibition for tumor regression in vivo, these in vitro data suggest that concomitant PI3Ki treatment activates non-tumor cell autonomous, TAM-mediated innate immunity via a DNA-damage independent mechanism in Myc-CAP (STING^{lo}) tumors.

To elucidate the mechanism by which PARPi/PI3Ki induces STING pathway activation within TAMs, the hypothesis that rucaparib/buparlisib-induced DNA DSBs within tumor cells cross-talks via MVs that activate cGAS/STING pathway within TAMs in the TME was tested. First, the quantity and cargo content of MVs isolated from B6-Myc (STING^{hi}) and Myc-CAP (STING^{lo}) cells treated with rucaparib, singly and in combination with buparlisib, was evaluated. It was observed that the MVs ranged from 40-100 nm in size, based on Nanoparticle Tracking Analysis (NTA) measurements. Furthermore, the quantity of DNA DSBs associated with MVs was directly proportional to the intracellular DNA damage content, as assessed by Nanodrop® and p-TH2AX foci quantification, respectively, for both B6-Myc (STING^{hi}) and Myc-CAP (STING^{lo}) cells treated with rucaparib and buparlisib, singly and in combination (FIGS. 17A, 29A, 29D, 29E).

Next, ex vivo assays were conducted using single-cell suspensions of Myc-CAP (STING^{lo}) tumors treated with exogenous rucaparib, singly and in combination with buparlisib. Interestingly, an increase in IFNβ production was observed within the supernatants of tumor allograft single cell suspensions following ex vivo rucaparib/buparlisib combination treatment, not observed with either single agent. Furthermore, the impact of these drug(s) was tested in the presence or absence of GW4869, an inhibitor of MV biogenesis and release. Concomitant ex vivo GW4869 treatment of tumor cell suspensions abolished the IFNβ production following rucaparib/buparlisib treatment (FIG. 29F), thus demonstrating that cGAS/STING pathway activation within the TME occurs via MV-associated DNA DSBs released from tumor cells.

If DNA DSB fragments are localized to the MV surface, resulting in activation of cGAS/STING pathway activation within TAMs, then co-culture of bone marrow derived macrophages (BMDMs) with DNase I treated supernatants from rucaparib/buparlisib-treated cancer cells would be predicted to result in abrogation of Type I IFN response relative to untreated MVs. On the other hand, if DNA DSB fragments are enclosed within MVs, then DNase I treatment would have no effect on induction of Type I IFN response within BMDMs. A striking decrease was observed of IFNβ release from BMDMs that were co-cultured with rucaparib/buparlisib-treated Myc-CAP supernatants that were treated with DNase I, relative to the corresponding supernatants that were not treated with DNase I (FIG. 29G), thus demonstrating that MV surface-associated (and not internalized) DNA DSB fragments are responsible for the Type I IFN response elicited within BMDMs.

Recent studies have demonstrated that direct transfer of cGAMP through tight junctions can activate the cGAS/STING pathway within the TME. To test the hypothesis that DNA DSBs, and not cGAMP, are responsible for cGAS/STING pathway activation within TAMs, BMDMs were co-cultured with recombinant cGAMP that was pre-treated with DNAse. Importantly, DNase I pre-treatment did not abrogate the Type I IFN production within BMDMs in response to cGAMP (FIG. 29H), demonstrating that it is the MV surface-associated DNA DSBs, not direct cGAMP transfer within the TME, that is responsible for cGAS/STING activation within TAMs.

To confirm that cGAS/STING pathway activation within TAMs is responsible for the Type I IFN response within the TME, supernatants from B6-Myc (STING^{hi}) and Myc-CAP (STING^{lo}) cancer cells treated with rucaparib and buparlisib, singly or in combination, were co-cultured with STING proficient and STING-deficient BMDMs (FIGS. 29I-29L). The rucaparib/buparlisib-induced IFNβ production observed when conditioned supernatants from B6-Myc (STING^{hi}) and Myc-CAP (STING^{lo}) cells were co-cultured with STING proficient BMDMs (FIGS. 29I, 29K) was abrogated under similar conditions with STING^{-/-} BMDMs (FIGS. 29J, 29L). To determine whether the DNA-associated MV release from tumor cells and activation of STING within TAMs is relevant in vivo, Myc-CAP (STING^{lo}) tumor-bearing mice were treated with the degerelix/rucaparib/buparlisib combination in the presence or absence of STING antagonist H-151 or MV biogenesis and release inhibitor GW4869. Critically, the anti-tumor response elicted by degarelix/rucaparib/buparlisib combination in Myc-CAP (STING^{lo}) tumor-bearing mice that were concomitantly treated with H-151 or GW4869 was abrogated (FIG. 29M) Collectively, these ex vivo and in vivo studies revealed that PARPi/PI3K treatment activates tumor-cell extrinsic cGAS/STING pathway within TAMs via MV surface-associated dsDNA cargo released from Myc-CAP (STING^{lo}) tumor cells.

Optimal STING Pathway Activation Within TAMs Requires Bth MV Surface-Associated DNA DSBs and PI3Ki-Mediated De-Repression of cGAS Activity.

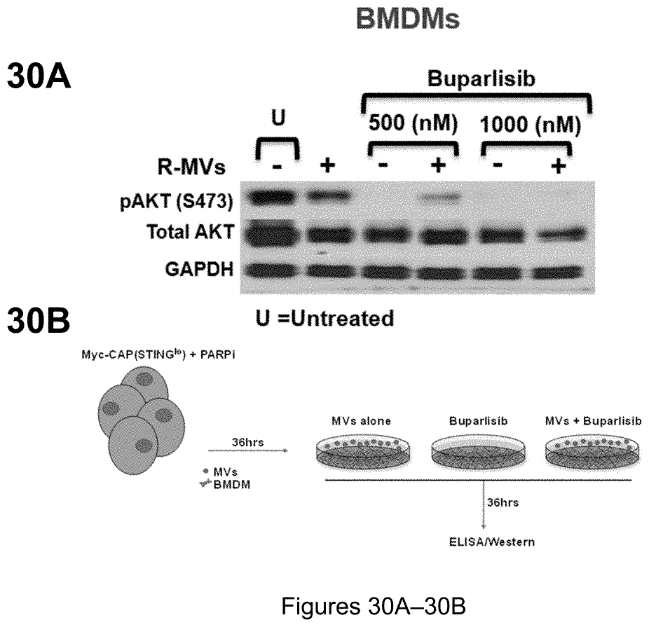

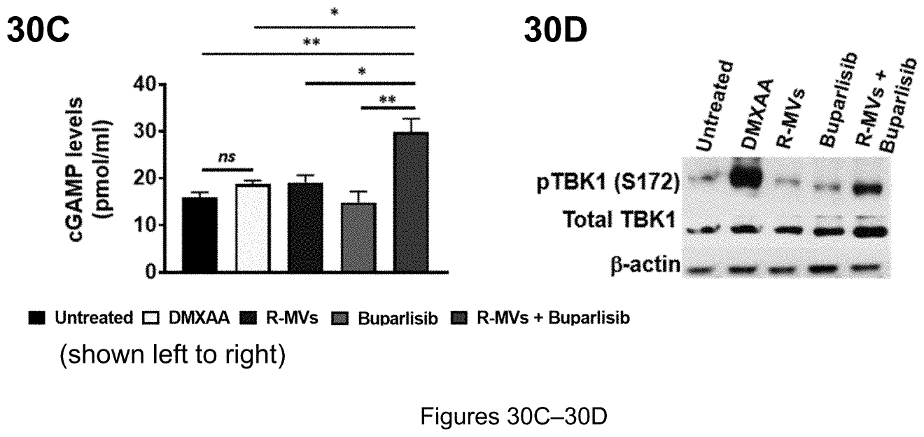

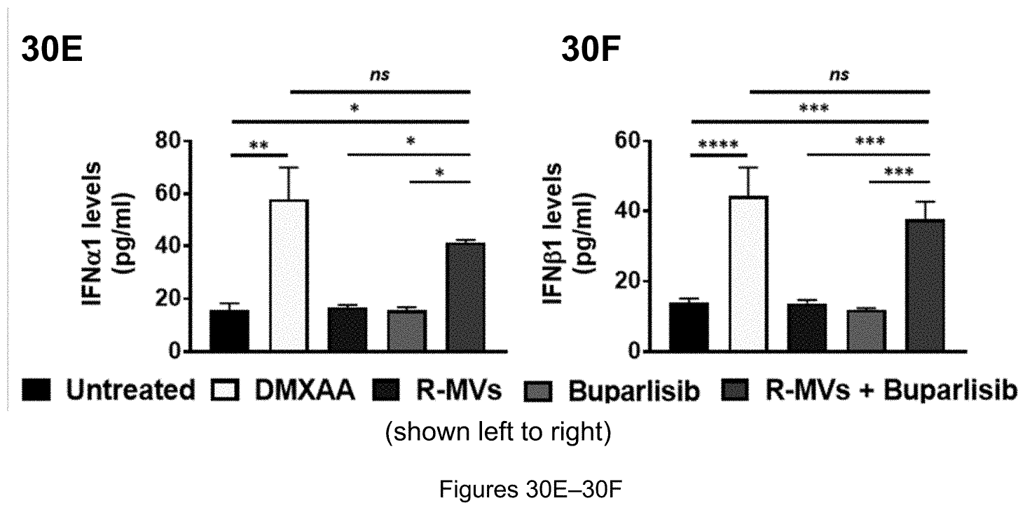

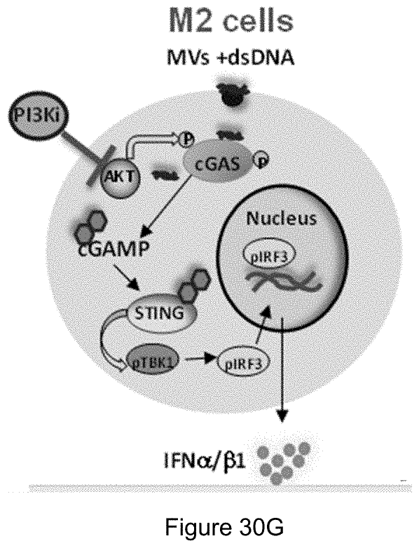

Recent work has shown that AKT can phosphorylate cGAS at Ser-291 and Ser-305, leading to post-translational suppression of its enzymatic activity. Furthermore, it was observed that concomitant PI3Ki did not induce additive DNA damage with PARPi in Myc-CAP (STING^{lo}) cells, suggesting that the addition of PI3Ki elicits a non-tumor cell autonomous DNA-damage independent mechanism for cGAS/STING pathway within TAMs. It was hypothesized that the requirement for concomitant PI3Ki treatment to activate DNA-damage induced STING pathway activation within TAMs stems from its ability to de-repress cGAS enzymatic activity, resulting in increased production of STING ligand 2′3′-cGAMP (cyclic guanosine monophosphate-adenosine monophosphate). To specifically test this hypothesis, Myc-CAP (STING^{lo}) cells were treated with rucaparib in vitro for 36 hours, followed by isolation of MVs from supernatant (R-MVs). Next, was BMDMs were co-cultured with R-MVs in the presence or absence of buparlisib (at target inhibitory concentration of 1 FIG. 30A) for 36 hours and levels of intra-cellular cGAMP were measured within macrophages by ELISA. Interestingly, a significant induction of cGAMP was observed only with the combination of buparlisib and MVs derived from rucaparib-treated Myc-CAP (STING^{lo}) cells, not corresponding single-agent controls (FIG. 30C). Critically, the combination of buparlisib and R-MVs resulted in downstream activation of STING signaling pathway components (FIG. 30D) and a significant incrase in IFN-α (FIG. 30E) and IFN-β (FIG. 30F), similar to what was achieved with direct treatment of BMDMs with STING agonist DMXAA (FIGS. 30E-30F) Taken together, these data demonstrate that optimal STING activation within macrophages requires both PARPi-mediated MV surface-associated DNA DSBs and PI3Ki-induced de-repression of cGAS enzymatic activity (FIG. 30G).

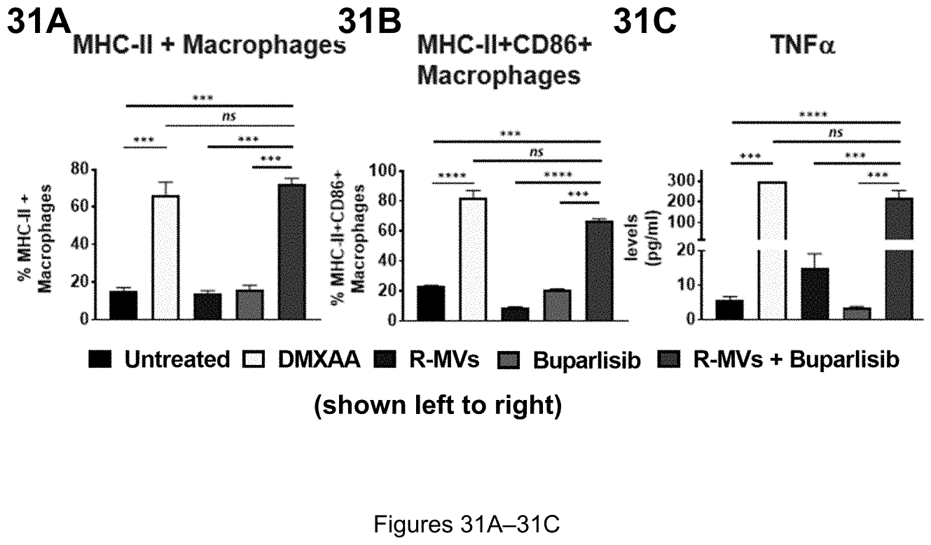

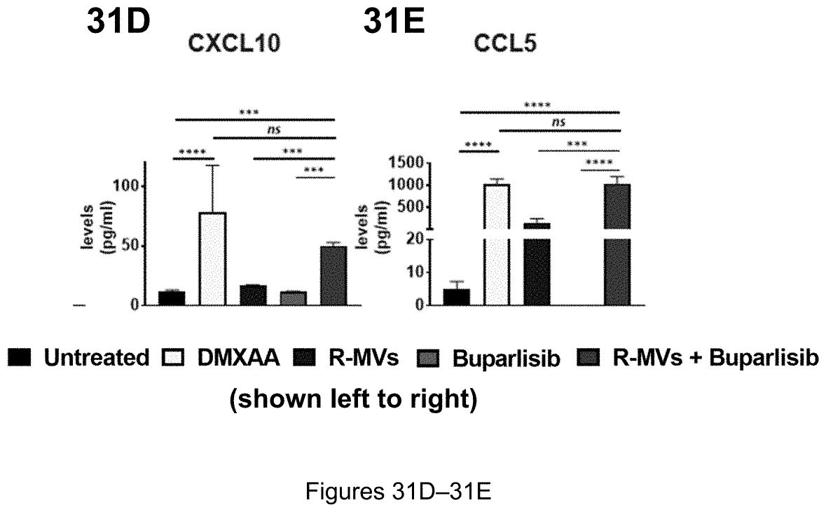

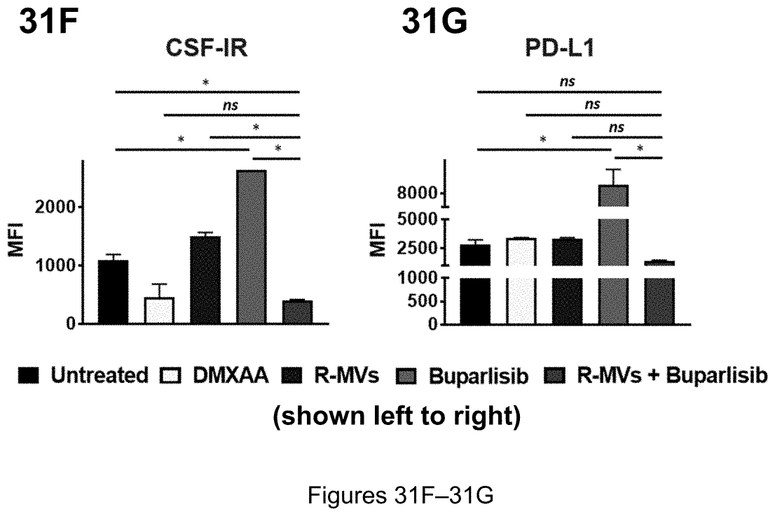

To determine whether PARPi/PI3Ki-induced cGAS/STING pathway activation within macrophages results in their polarization from pro-tumorigenic M2 to anti-tumorigenic M1 phenotype, BMDMs were co-cultured with buparlisib, singly or in combination with purified MVs derived from supernatants of Myc-CAP (STING^{lo}) tumor cells that were treated with rucaparib (R-MVs). Flow cytometry analysis revealed an increase in MHC Class II and CD86 expression within macrophages, indicating enhanced activation and antigen presenting capacity, respectively, following treatment with both buparlisib and R-MVs, relative to single agent buparlisib or R-MVs controls. Treatment of BMDMs with DMXAA achieved similar levels of macrophage activation and antigen presentation, relative to buparlisib/R-MVs combination (FIGS. 31A-31B). Furthermore, an increase in TNF-α and T cell chemoattractant chemokines CXCL10 and CCL5 release (FIGS. 31C-31E) and reversal of CSF-1R and PD-L1 inhibitory marker expression (FIGS. 31F-31G) were observed within BMDMs co-cultured with both buparlisib and R-MVs, relative to single-agent controls, thus demonstrating M1 polarization following R-MVs and buparlisib combination treatment.

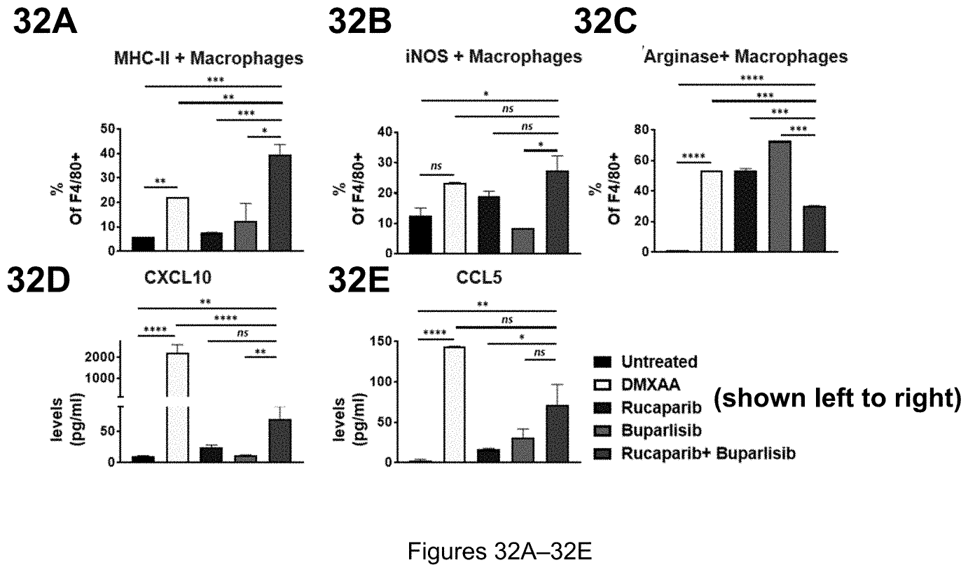

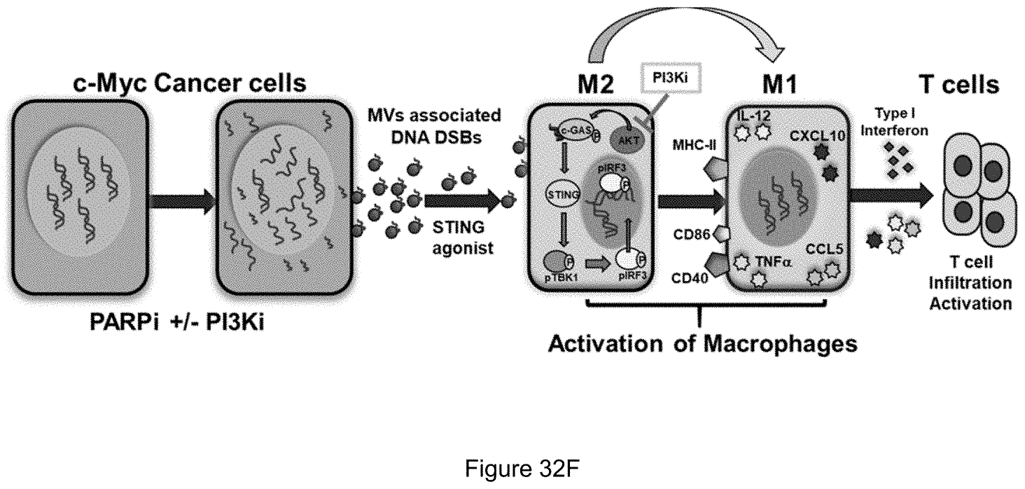

Analogous co-culture experiments of BMDMs with supernatants derived from rucaparib-treated Myc-CAP (STING^{lo}) cells, in the presence or absence of buparlisib, were also performed. Strikingly, flow cytometry analysis revealed increased MHC Class II and iNOS expression, and decreased Arginase I expression only with the rucaparib/buparlisib combination, and not corresponding single-agent controls (FIGS. 32A-32C). Furthermore, cytokine profiling of supernatants from ex vivo experiments revealed an increase in CXCL10 and CCL5 released from BMDMs that were treated with the supernatants derived from rucaparib/buparlisib treated Myc-CAP (STING^{lo}) cells, not corresponding single agent controls (FIGS. 32D-32E). Taken together, these results demonstrate that PARPi-induced DNA DSBs and PI3Ki-induced cGAS de-repression within TAMs is required for optimal cGAS/STING activation and re-programming of macrophages from M2 suppressive to M1 activated, anti-tumor phenotype within the TME. A mechanistic model summarizing the findings is depicted in FIG. 32F.

### Discussion

Androgen receptor (AR)-directed therapies have had incremental benefit, but are generally not curative for the treatment of metastatic PC. There has been renewed interest in PC immunotherapy, partly based on the profound and durable clinical responses to ICB antibodies targeting CTLA-4 and PD-1/PD-L1 in other cancers. While de novo androgen deprivation therapy (ADT) can induce immune cell infiltration within non-flamed tumor microenvironment of PC, only 10-25% of mCRPC patients respond to ICB. Here is shown a paucity of immune cell infiltrate within the TME in mCRPC patients, which is one mechanism of resistance to ICB. Furthermore, it is demonstrated that the majority of immune cells within the TME of mCRPC patients are comprised of TAMs (see e.g., FIGS. 13A-13L). On the basis of these findings, it was hypothesized that activation of innate immunity within TAMs will enhance immune-responsiveness in PC. Critically, this work demonstrates that targeting both fundamental DNA repair and oncogenic signaling pathways could markedly increase responsiveness to ICB via activation of the cGAS/STING pathway within TAMs.

While PARPi have been FDA approved in BRCA ½-mutated breast cancer, ovarian cancer, mCRPC, and pancreatic cancer, it has limited efficacy in non-BRCA HR-deficient mCRPC as well as HR-proficient cancers. Here it is demonstrated that PARPi, either singly and/or in combination with PI3Ki, can induce DNA damage in HR-proficient c-myc driven murine PC models (FIGS. 17A-17C, FIGS. 29A-29D). This result is likely related to enhanced dependency of c-myc-induced replicative stress on PARP-1 mediated DNA repair, with resultant downregulation of the CDK18/ATR axis. Consistent with clinical observations, PARPi-induced DNA damage is insufficient to induce apoptosis of HR-proficient c-myc-driven cancer cells in vitro (FIGS. 17A-17C, FIGS. 29A-29D).

The cGAS/STING pathway is physiologically activated by cytosolic double-stranded DNA (dsDNA), which typically occurs in the context of viral infections. Cyclic GMP-AMP synthase (cGAS) is a primary cytosolic dsDNA sensor that generates cyclic dinucleotides (cGAMP), which acts as a second messenger to activate STING, which in turn induces the recruitment of TBK1 and IRF-3 to form a complex with STING. The activation of IRF-3 and/or NF-κB signaling pathways induce the expression of Type I IFNs and pro-inflammatory cytokines. Recent murine studies have demonstrated that PARPi can activate tumor cell-intrinsic cGAS/STING pathway in murine HR-deficient breast and ovarian cancers, resulting in anti-tumor responses that can be accentuated with PD-1 blockade. This has led to several clinical trials evaluating radiotherapy or PARP inhibitors with ICB in different solid tumor malignancies. However, clinical trial data and preclinical studies described here demonstrate that PARPi is insufficient to drive tumor cell-extrinsic cGAS/STING pathway activation and does not enhance ICB responsiveness in a co-clinical trial testing PARPi/ICB combination in HR-proficient mCRPC patients and c-myc-driven murine models of prostate cancer (FIGS. 16A-16B, 19A-19E, 25A-25B, 26C-26D, 27B, 27D). Critically, concomitant PARPi/PI3Ki activates tumor cell-extrinsic cGAS/STING pathway activation within M2 TAMs, resulting in their polarization into an anti-cancer M1 phenotype, and T cell infiltration/activation and tumor regression in immune-refractory HR-proficient c-myc-driven models of PC (FIG. 32F). This PARPi/PI3Ki-mediated tumor regression in vivo is abrogated with systemic macrophage depletion, demonstrating that the reprogramming of TAMs is responsible for driving anti-cancer innate immunity. Furthermore, the PARPi/PI3Ki combination fails to induce apoptosis of B6-Myc (STING) and Myc-CAP (STING^{lo}) cancer cells in vitro, and effectively control tumor growth in vivo in corresponding immunodeficient models. Collectively, these findings demonstrate that PARPi/PI3Ki combination exerts its anti-cancer activity primarily via a non-tumor cell autonomous, innate immune, macrophage-driven mechanism. Given this unanticipated immune-based mechanism for PARPi/PI3Ki combination in c-myc-driven PC, these data highlight the critical unmet need for the development of more sophisticated ex vivo and in vivo combinatorial drug screening platforms, which incorporate immunological readouts beyond apoptosis induction of cancer cell lines in vitro.

Consistent with prior studies that have shown that PARPi, in combination with PI3Ki, induces additive DNA damage and suppresses tumor growth in breast and prostate preclinical models, additive DNA damage was observed in B6-Myc (STING) tumors with rucaparib in combination with buparlisib. In contrast, the addition of buparlisib to Myc-CAP) (STING^{lo}) cells did not further increase DNA damage, relative to rucaparib alone, thus suggesting that concomitant PI3Ki treatment elicits anti-cancer effects via a DNA-damage independent mechanism in Myc-CAP (STING^{lo}) tumors. A recent study has demonstrated that AKT phosphorylates the S291 or S305 of the carboxyl-terminal enzymatic domain of mouse or human cGAS, respectively, and that this phosphorylation robustly suppresses cGAS enzymatic activity, leading to decreased cytokine production and antiviral activity following DNA virus infection. Furthermore, the ex vivo studies revealed that presence of DNA DSBs within the TME is insufficient to drive cGAS/STING pathway activation within TAMs. The hypothesis tested is that the PI3K/AKT pathway suppresses c-GAS enzymatic within TAMs, thereby preventing STING pathway activation in response to PARPi-induced DNA DSBs. Consistent with this hypothesis, it was observed tht PI3Ki de-represses cGAS enzymatic activity, resulting in increased cGAMP production, STING activation and Type I IFN production, which is required for DNA DSB induced cGAS/STING activation within TAMs.

In this study, the exciting observation was made that PARPi-induced DNA DSBs are transported within the TME as cargo associated with the surface of MVs, which can secondarily activate cGAS/STING pathway in TAMs. Furthermore, this DNA DSB-induced cGAS activation within TAMs is abolished by concomitant DNase I treatment, suggesting that the DNA DSB is associated on the surface of the MVs, and not internally within the membrane lipid bilayer. These findings are supported by a recent study has shown that dsDNA can be associated with the surface of exosomes. Given that exosomes/MVs can have immunosuppressive and pro-metastatic properties, the findings suggest the possibility that PARPi can render MVs more immunogenic, similar to prior observations made with MVs produced after radiotherapy. These findings suggest utilizing blood-based MVs biomarkers as pharmacodynamic readouts of PARPi-induced DNA damage within the TME.

Prior studies have demonstrated that early stages of castration induce T-effector and T-regulatory cell infiltration within human and prostate tumors. In this study, two different murine models of c-myc-driven PC were interrogated: B6-Myc (STING^{hi}) and Myc-CAP (STING^{lo}), which express high and low levels of cGAS/STING signaling pathway components (FIG. 15), respectively, and mimic the heterogeneity of cGAS and STING expression observed in human PC (FIG. 14). In the B6-Myc (STING) context, the combination of PARPi/PI3Ki was sufficient to drive a macrophage-mediated innate immune response and tumor clearance, whereas Myc-CAP (STING^{lo}) bearing syngeneic mice were de novo resistant to this combination. Strikingly, it was observed that the addition of ADT, when combined with PARPi/PI3Ki, resulted in an anti-cancer innate immune response and tumor clearance, similar to that observed in B6-Myc mice treated with the combination without castration. There are several potential explanations for these findings. First, C57BL6 is a more immunogenic strain than FVB mice, resulting in a lower threshold for immune-sensitization that is dependent on host factors. Second, the presence of higher tumor cell intrinsic STING levels in B6-Myc, relative to Myc-CAP cells, could account for the approximately 10-fold higher baseline CD45+ immune cell infiltration in B6-Myc tumors in vivo, relative to Myc-CAP tumors. Following castration, Myc-CAP (STING^{lo}) tumors have an approximately 5-fold increase in CD45+ immune cell infiltration (predominantly TAMs), which provides the necessary immunological milieu needed for optimal cGAS/STING activation within the TME following PARPi/PI3Ki treatment. Collectively, these data suggest that baseline cGAS and STING expression can be developed as potential biomarkers for response to PARPi/PI3K combination therapy in earlier stages of PC treatment, where castration is not standard-of-care. Given the findings that high-risk (Gleason≥8) PC is enriched for tumors with low tumor cell intrinsic STING expression, the findings in this paper warrant the development of immuno-oncology clinical trials testing the combination of ADT with PARPi/PI3Ki/PD-1 blockade in de novo hormone-sensitive, locally advanced or metastatic PC.

In summary, these studies have demonstrated that concomitant targeting of PARP and PI3K signaling pathways can trigger non-tumor cell autonomous c-GAS/STING pathway activation within TAMs, thereby enhancing T cell recruitment/activation into the TME and tumor regression in HR-proficient c-myc-driven murine models. Based on these findings, PARPi/PI3Ki combination therapy could markedly increase the fraction of PC patients responsive to ICB, independent of HR status, and clinical trials to test this combinatorial approach are warranted.

### Example 3

### Study of PTEN Deficient Cancers

### Methods

Bioinformatic analysis of gene signature using TCGA and Stand Up to Cancer databases. Determination of PTEN loss: RNA-seq expression data was obtained from primary prostate cancer cohort within the TCGA (n=437 prostate cancer patients, n=6153 total cancer patients). Patients were categorized into non-T-cell inflamed vs. T-cell inflamed groups with high DDR signature, as determined previously (Walker S M, Knight L A, McCavigan A M, Logan G E, Berge V, Sherif A, et al. Molecular Subgroup of Primary Prostate Cancer Presenting with Metastatic Biology. Eur Urol 2017; 72(4):509-18). The RNA-seq read counts derived from the TCGA cohort for the patients classified into different clusters were used to estimate differential expression of a targeted panel of genes between the different clusters. The differential expression analysis was done using DESeq, and a FDR adjusted p-value cut-off of 0.05 was selected along with a log fold-change cutoff of 1.5. Statistical analysis for changes in gene expression of PTEN, between the different clusters was done using unpaired t-test and Fisher's exact test. Defining MDSCs and T cell inflammed signatures: Metastatic prostate tumors (n=43, Stand Up to Cancer) were differentiated on the basis of genomic loss of PTEN (at least one copy), and then clustered based on an MDSC-high or MDSC-low gene-expression signature, comprising of 14 genes. PTEN-deficient tumors of all histologies from the TCGA database (n=6153 from 30 different tumor histologies), were stratified further, based on a pre-determined T cell-inflamed or non-T cell-inflamed gene signature.

Cancer Cell Lines and in vitro drug treatments. Transgenic c-myc^{hi} prostate tumor derived cell line, Myc-CAP and TRAMPC1, ovarian cancer cell line ID8 and murine colon adenocarcinoma line MC38 were obtained from the American Type Culture Collection (ATCC) and passaged in either 1×RPMI/1×DMEM (with phenol) or 1×DMEM (without phenol red) containing 10% fetal bovine serum, 1% penicillin-streptomycin, 2% L-glutamine and 1×ITS. ID8^{pten-/-p53-/-}cells were a kind gift from Dr. Ian McNeish (Imperial College, London). For generation of STING over expressing cell lines −Myc-CAP^{pten+/+} and ID8^{pten-/-} cells were transfected with commercial available plasmid (DC-Mm22462-SH02, Genecoepia™), sorted and expanded as per protocol. For generation of pten deficient cell lines, the CRISPR/Cas9 system was used to disrupt the expression of the PTEN gene as described previously (Ran et al. Nat Protoc 2013). For transient knock down of PTEN—0.3 million cells were seeded in 12 well plates and treated with directed siRNA sequences (Genecoepia™) for 24 hours prior to drug treatments. For In vitro drug treatments, the following concentrations were used: DMXAA (Sigma Aldrich—50 μg/mL), Rucaparib (Clovis Oncology®—500 nM), Buparlisib (the Stand up to Cancer Drug Formulary at Dana Farber Cancer Institute—1 μM), GSK'771 (GlaxoSmithKline®—1 μM), Copanlisib (Selleck Chem, 500 nM), CAL101 (the Stand up to Cancer Drug Formulary at Dana Farber Cancer Institute—500 nM), IPI549 (Selleck Chem—500 nM). All drugs were reconstituted in DMSO and further diluted in 1×PBS for in vitro applications.

Generation of bone marrow derived macrophages. Bone marrow derived macrophages were differentiated as previously described (Weischenfeldt J, Porse B. Bone Marrow-Derived Macrophages (BMM): Isolation and Applications. CSH Protoc 2008; 2008:pdb prot5080). Briefly, bone marrow cells were isolated from male FVB/NJ mice and differentiated in the presence of 1×RPMI media (supplemented with 10% fetal bovine serum, 1% penicillin-streptomycin and 2% L-glutamine) containing 30% L-conditioned media or M-CSF (50 ng/mL) for 5-7 days. Differentiated cells were stimulated directly with 50 μg/mL of 5,6-Dimethylxanthenone-4-acetic Acid (DMXAA, mouse STING agonist) for 36 hours. Following treatment, supernatants were collected for Type I IFN ELISA (LEGEND MAX™ Mouse IFNβ1 ELISA, Biolegend®) and processed as specified in protocol.

In vivo drug efficacy studies using murine tumor models. Wild-type (WT) C57BL/6J, FVB/NJ mice were purchased from Jackson laboratories and mice were kept in an AALAC (American Association for the Accreditation of Laboratory Animal Care) certified barrier facility at the University of Chicago. Animal work was carried out according to approved IACUC (Institutional Animal Care and Use Committee protocols). For Myc-CAP cell line based experiments, mice aged 8-10 weeks were engrafted with 1 million cells re-suspended in 1×PBS, under anesthesia. Treatments were started when tumor volumes reached approximately 250-400 mm³, and mice were randomly allocated to treatment groups as indicated. Tumor volume was calculated using the formula: 0.5×longest diameter×(shortest diameter)². Euthanasia was performed for tumor ulceration and/or tumor diameter >2 cm, as per IACUC-approved protocol. All in vivo treatments were done for 20-23 days and tumor volume measurements were collected on a daily basis. For ID8 tumors, the weights (gm) as well as the abdominal girth (mm) measurements were recorded daily. Drug treatment was started when the girth measurement was at 24 mm. The mice were monitored daily for signs of distress/ill health and sacked when diameter reached 35 mm and the weight was >30 gm. For in vivo treatments, lyophilized drugs were reconstituted in appropriate solvents and were administered at the following doses: degarelix (0.625 mg/kg) single dose i.p. Rucaparib at 150 mg/kg, buparlisib at 30 mg/kg were done daily. STING agonist 5,6-dimethylxanthenone-4-acetic Acid (DMXAA) was injected intra-tumorally, once at a dose of 500 μg/kg. Anti-PD-Ll antibody was given i.p. once every 3 days, for duration of treatment.

Flow Cytometry. Murine tumors: Murine tumors were processed into single cell suspensions via gentle mechanical dissociation in 6 well plates containing 1 mL of 10% RPMI media supplemented with 10% fetal bovine serum, 1% penicillin-streptomycin and 2% L-glutamine. Cell suspensions were centrifuged at 500×g for 5 minutes at 4° C. and were passed through a 70-micron mesh, prior to re-suspension in FACS buffer (1×PBS containing 0.5% FBS and 0.01% sodium azide). For the stain, one million cells resuspended in 1×FACS buffer, were incubated with titrated concentrations of anti-mouse antibodies (Biolegend®), namely, CD45, CD11b, CD11c, CD19, F480,Ly6G, Ly6C, PD-L1, VISTA, I-A^{e}/IA^{b}, H-2K^{b}, CD3, CD4, CD8, 4-1BB, ICOS, and PD-1. Incubation with antibodies was done at 4° C. for 30-40 minutes. Following staining, cells were washed twice with 1× FACS buffer and fixed with 300 microliters of 4% paraformaldehye (Fisher Scientific®). Intracellular stains were done following fixation and permeabilization by staining with anti-mouse FOXP3 (Biolegend®) as per manufacturer's protocol. Analysis of cellular stains were done using BD instrument LSR 4-15 Fortessa™. Data files collected using BDFACSDIVA software were analyzed using FlowJo® software (Tree Star).

Cytokine Array/IFNβ1 ELISA. Supernatants from untreated or drug treated cancer cell lines and bone marrow derived macrophages were collected at 36 hours and spun down at 2000×g for 5 min at 4° C. to remove cellular debris. These clear supernatants were processed for detection of Type I Interferon by ELISA (LEGEND MAX™ Mouse IFNβ1 ELISA, Biolegend®) or for determination of a panel of cytokines/chemokines using LEGENDplex™ array (Mouse Anti-Virus Response Panel, Biolegend®) as per instructions in protocol provided.

Western blotting. RIPA buffer (Boston BioProducts) supplemented with protease (Roche®) and HALT phosphatase inhibitor cocktail (Thermo Scientific®) was used for preparation of total cell lysates. For western blotting, the following antibodies were used from Cell Signaling Technology®: Polyclonal rabbit anti-mouse-, phospho-AKT, total AKT, c-GAS, STING, phospho-IRF3, total IRF3, phospho-TBK1, total TBK1, PTEN, phospho NF-kB, total NF-kB, p53, lamin B1, β-actin, alpha-tubulin, and GAPDH. Images of scanned blots were processed using ADOBE Photoshop.

Ex vivo Assays. Subcutaneous tumors were isolated by gentle mechanical dissociation in the presence of 1×RPMI (supplemented with 10% fetal bovine serum, 1% penicillin-streptomycin and 2% L-glutamine). 1×ACK was used for RBC lysis. Single cell suspensions were washed twice with media, by centrifuging at 500×g for 5 min at 4° C. and quantified using 0.1% Trypsin solution. Cells were seeded at a concentration 0.2-0.5 million cells/mL and treated with indicated drugs singly or in combination for 36 hours. DMXAA (positive control) was used at indicated concentration. Supernatants from treated BMDMs were collected at the end of 36 hours and processed as per ELISA protocol for detection of cytokines/chemokines.

Microvesicle Isolation (MVs)/DNA extraction. Cells were treated for 36 hours with the indicated drugs, and supernatants were harvested and then centrifuged at 300×g for 5 minutes at 4° C. to pellet cells, This was followed by additional centrifugation steps at 2,000×g for 10 min at 4° C. to eliminate dead cell debris and at 10,000×g for 30 min in at 4° C. to remove larger vesicles. The supernatant was then collected and subjected to 100,000×g centrifugation in a Type 60 Ti rotor (38000 rpm) for 70 min at 4 ° C. The 100,000 g pellet was suspended in 1×PBS in a volume equal to the initial volume of supernatant (2 mL), and washed by an additional spin in the ultracentrifuge for 70 min at 4° C. The final EV pellet was collected in 1×PBS and used for quantification. Measurements of particle size distribution (PSD) and concentration were performed with a Nanosight® LM10 HS-BF instrument (Nanosight Ltd, UK), based on a Nanoparticle Tracking Analysis (NTA), using a 405 nm 65-mW laser and an EMCCD Andor® Luca camera. Samples were diluted with particle-free PBS in a 1:100 dilution (pH=7.4) to reach the optimal concentration for NTA. All measurements were performed under the identical camera settings (Shutter: 850, Gain: 450, Lower Threshold: 910, Higher Threshold: 11180, 60 s) and processing conditions (NTA 2.3 build 0033, Detection Threshold: 9 Multi, min Track Length: Auto, min Expected Size: 30 nm), Measurements were performed in multiple repeats (n=3) to collect an at least 5000 events, and then equal numbers of particles were used for downstream assays. Equal numbers of EVs were collected from each treatment group and DNA was extracted using Trizol® LS as per protocol (Thermo Fischer Scientific®) and then quantified using Nanodrop®.

qRT-PCR. Cancer cell lines: STING expression in PTEN proficient/deficient cell lines was assessed by qRT-PCR. Cells were used for isolation of RNA using Qiagen RNeasy® Plus isolation kit (Qiagen®). RNA isolated (100-500 ng) was used for cDNA synthesis (cDNA RT kit, Bio-Rad®), following which qRT-PCR was performed using SyBr Green Universal master mix (Bio-Rad®) and inventoried primers specific for mSTING, mIFNβ1. Each sample was analyzed in triplicate on ViiA™ 7 Real-Time PCR System (Applied Biosystems®). Data generated was normalized to GAPDH.

cGAMP assay. For the colorimetry-based detection of cGAMP production in M2 macrophages, cells were treated with EVs isolated from rucaparib-treated Myc-CAP cancer cells, in the presence or absence of buparlisib at indicated concentrations. Following 30 hours of treatment, the cells were harvested and cell lysates processed using recommended buffers (cGAMP detection kit, Cayman Chemical®) and then used for incubation with anti-cGAMP antibody and detection conjugate for 2 hours or overnight at 4° C. Next, substrate was added and cGAMP was detected at the indicated wavelength, as per manufacturer's instructions.

Viability Assays. Single cell suspensions/at a concentration of 0.5 million cells/0.5 mL of 1× Annexin Buffer or concentrated EVs, were stained with Annexin V/PI (FITC Annexin V Apoptosis Detection kit, BD Biosciences) as specified in manufacturer's protocol. Acquisition and analysis of the data sets were done as previously described in section on flow cytometry.

Image Stream Analysis. PTEN-proficient and deficient ID8 ovarian cancer cells were treated with DMXAA (50 ug/mL) for 0, 12, 24, and 36 hours. After termination at endpoints, cells were stained with primary antibodies—anti-mouse p-IRF3 (Cell Signaling Technology®), p-NFkB (Cell Signaling Technology®), followed by incubation with flurochrome tagged secondary antibodies and DAPI. Images of nuclear localization of p-IRF3 and p-NF-kB within single cells were acquired using ImageStreamg®^{x} Mark II (Amnis®) and data obtained was analyzed using IDEAS® software.

Statistical Analysis. One-way ANOVA/Mann Whitney/Unpaired t-test/Chi-Square tests were used for used for statistical evaluation of experimental datasets.

### Results

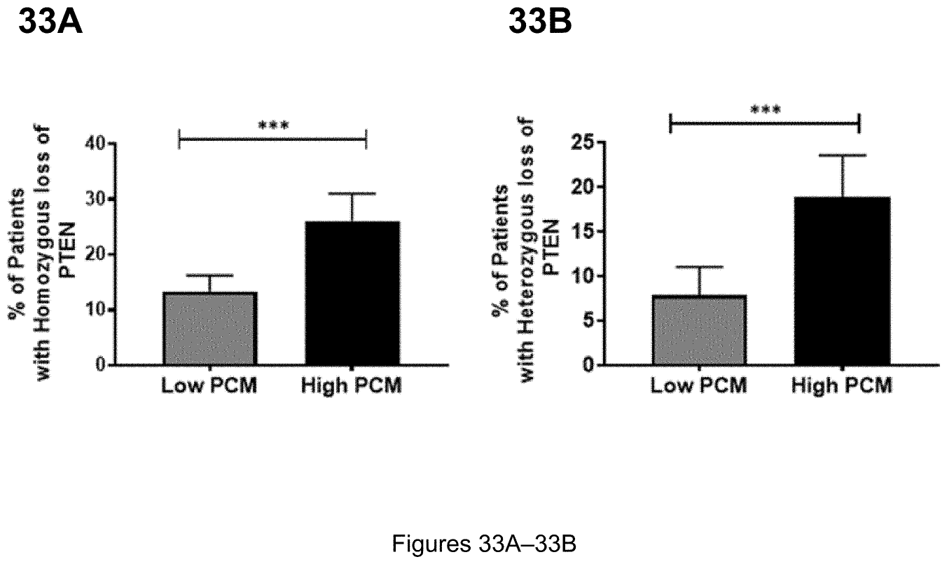

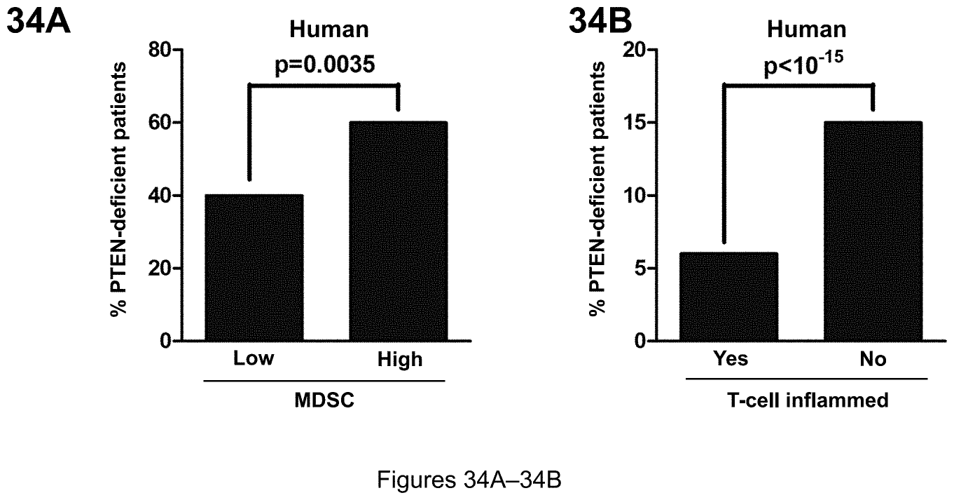

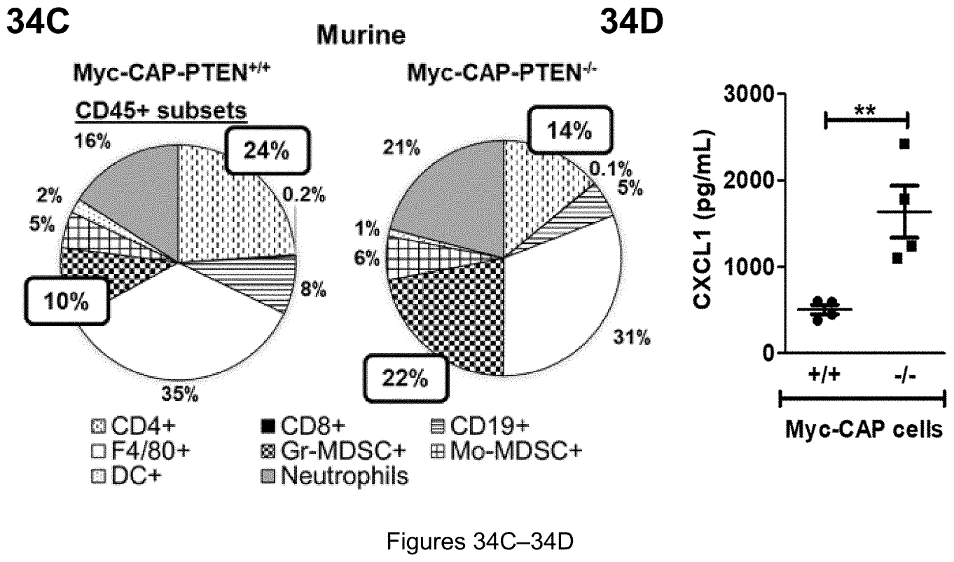

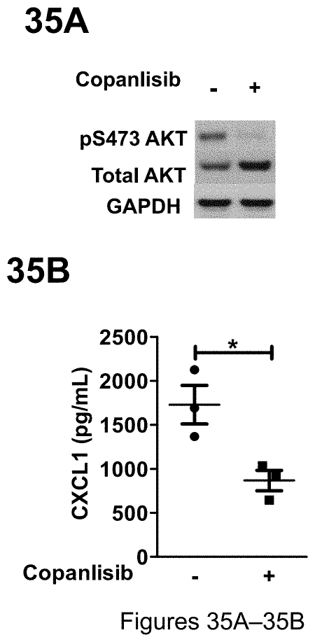

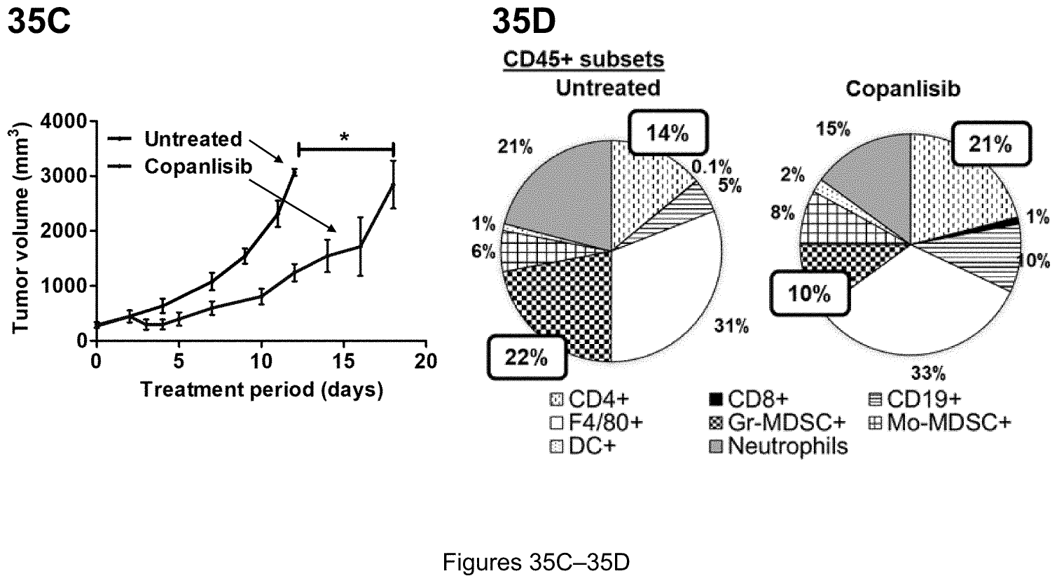

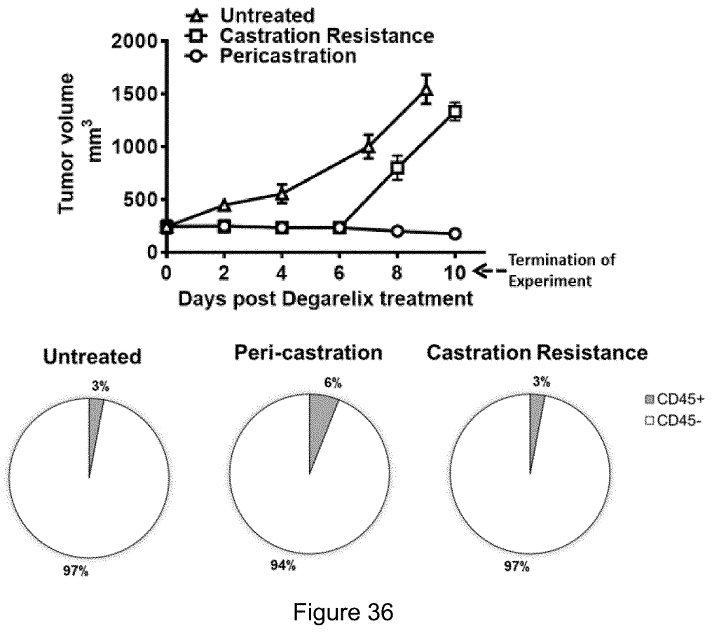

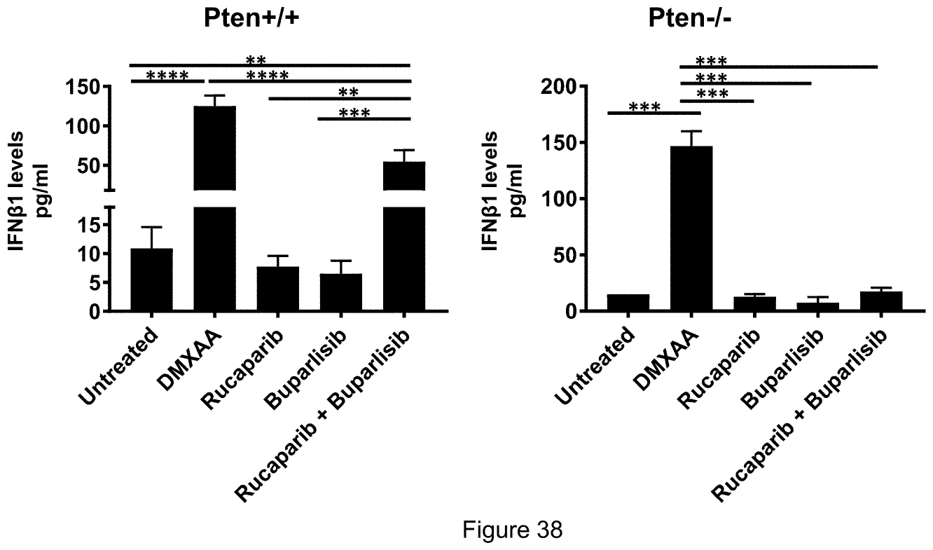

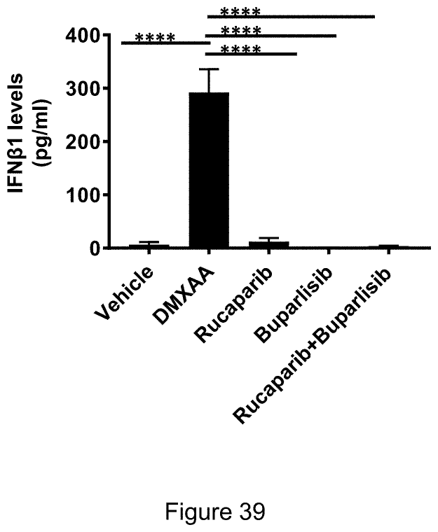

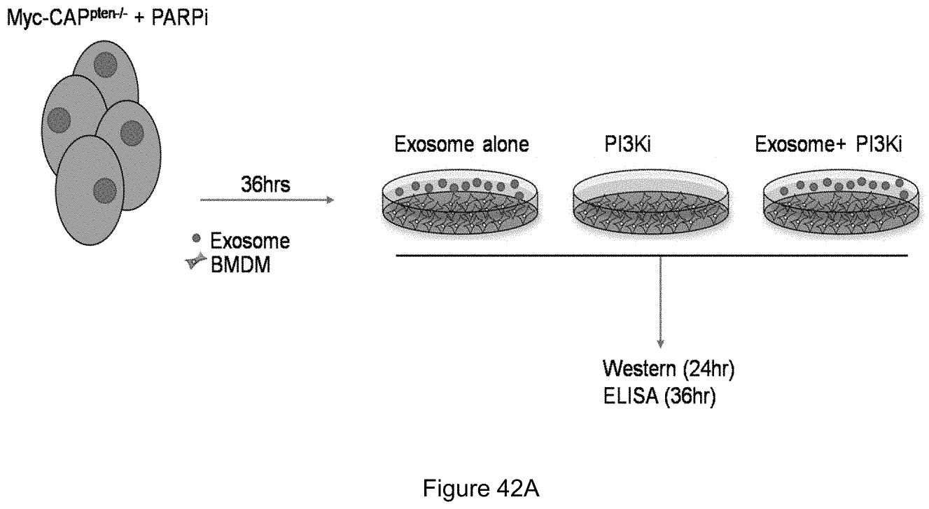

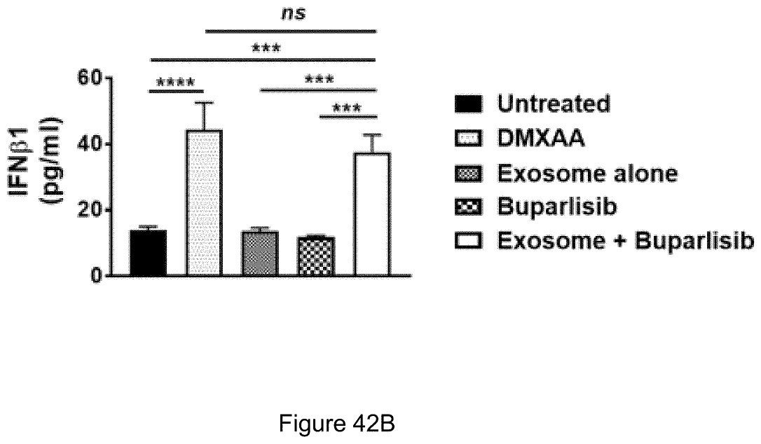

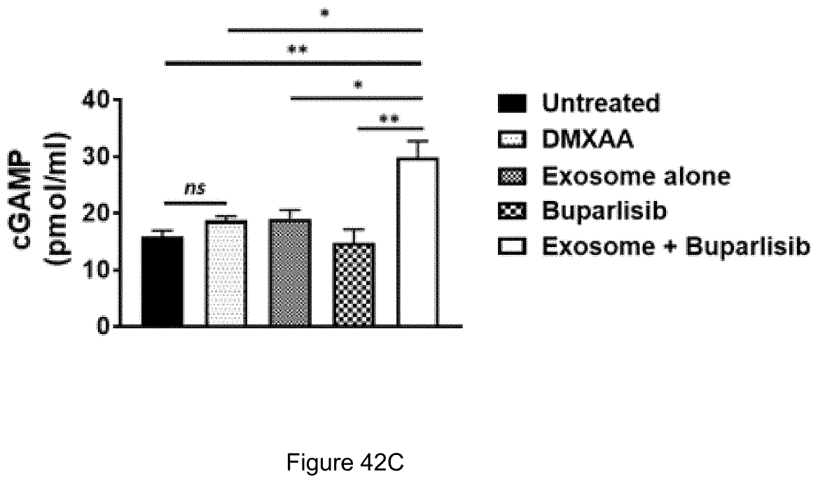

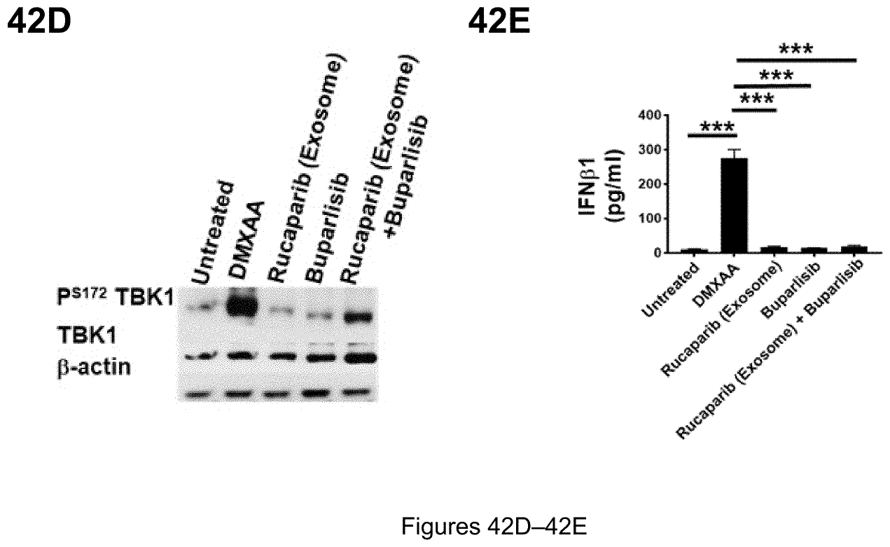

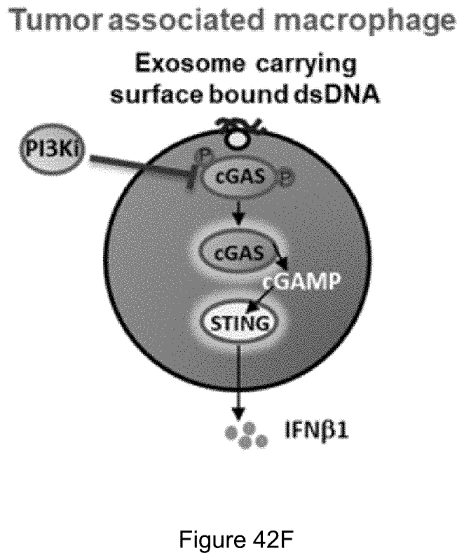

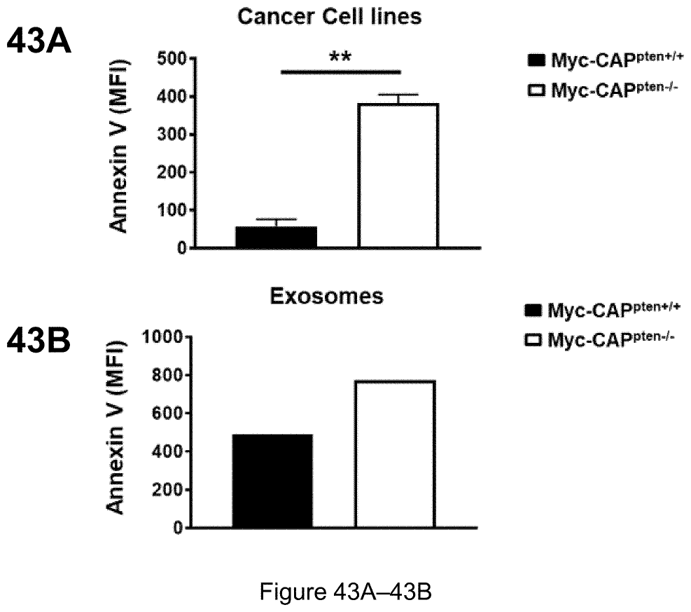

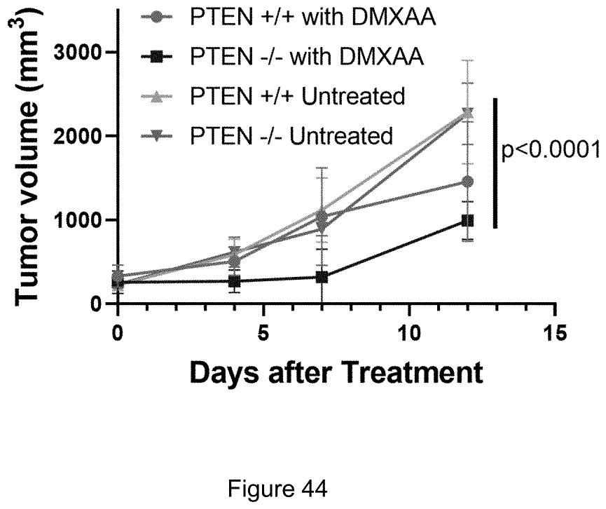

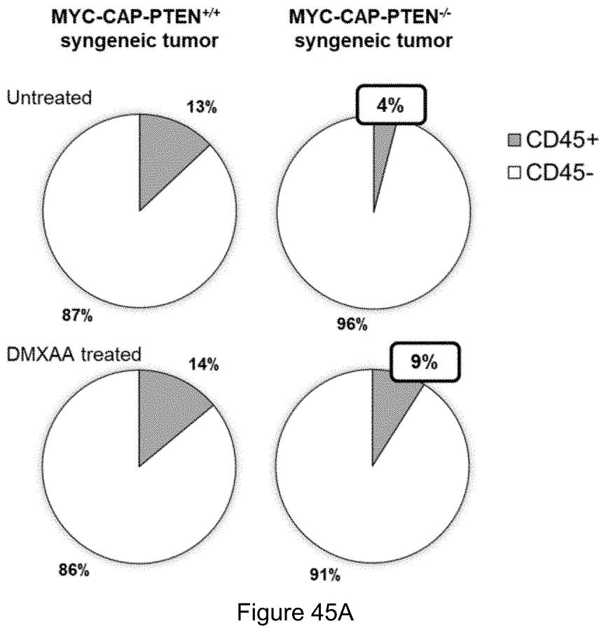

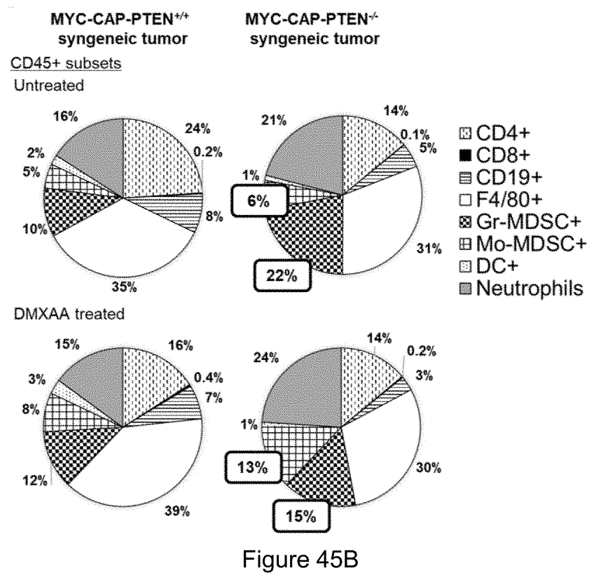

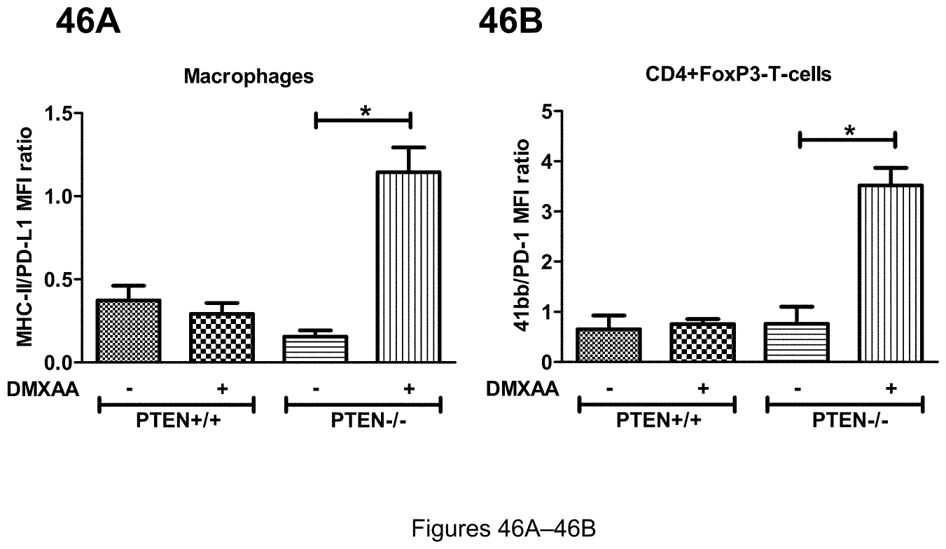

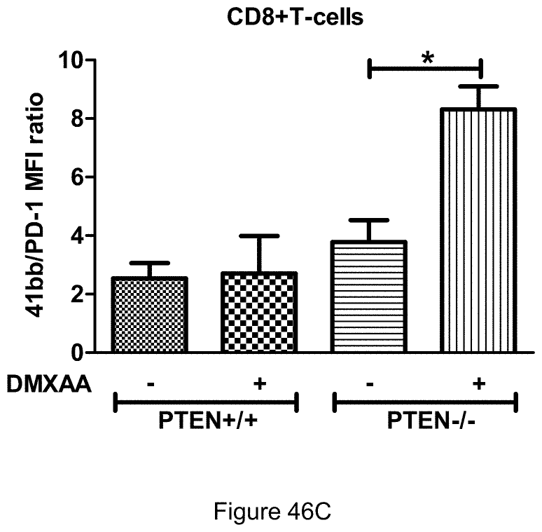

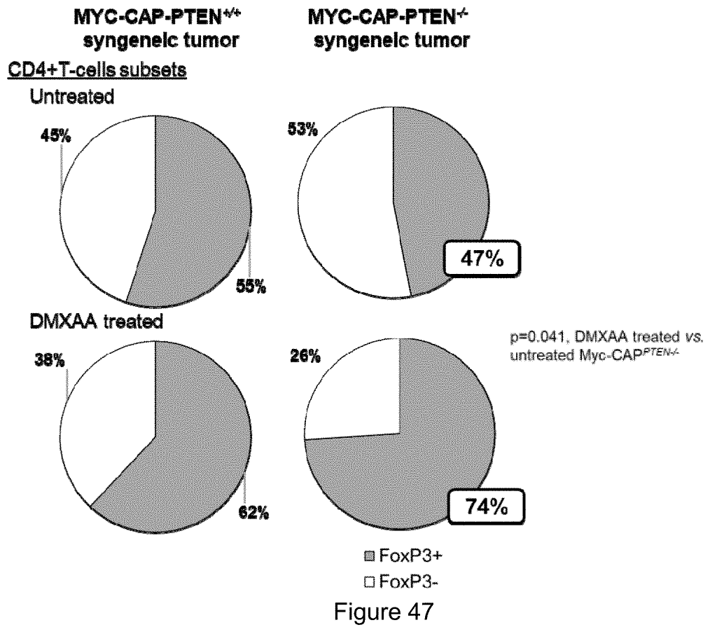

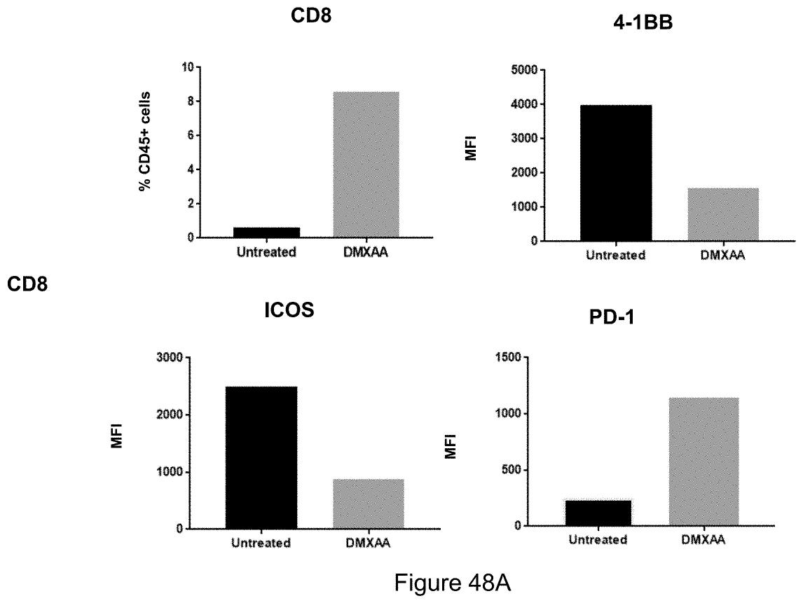

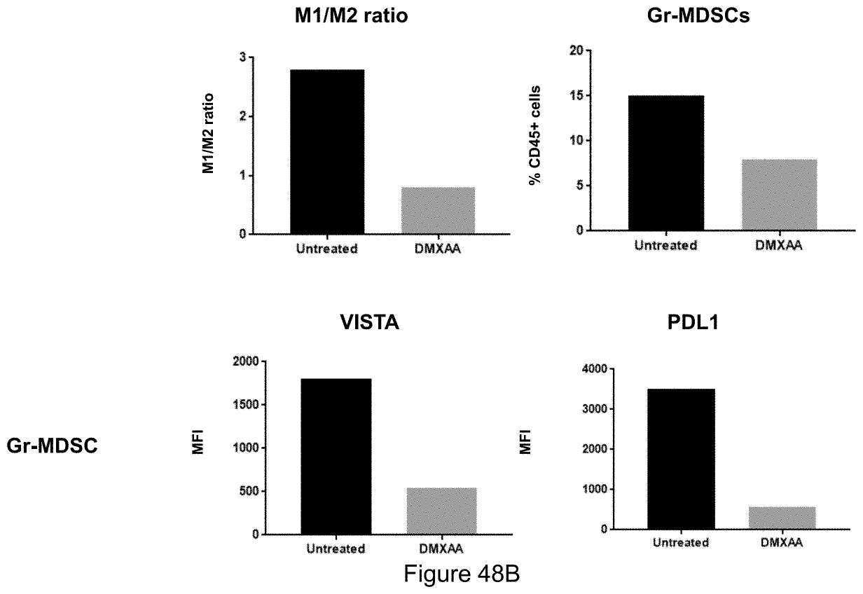

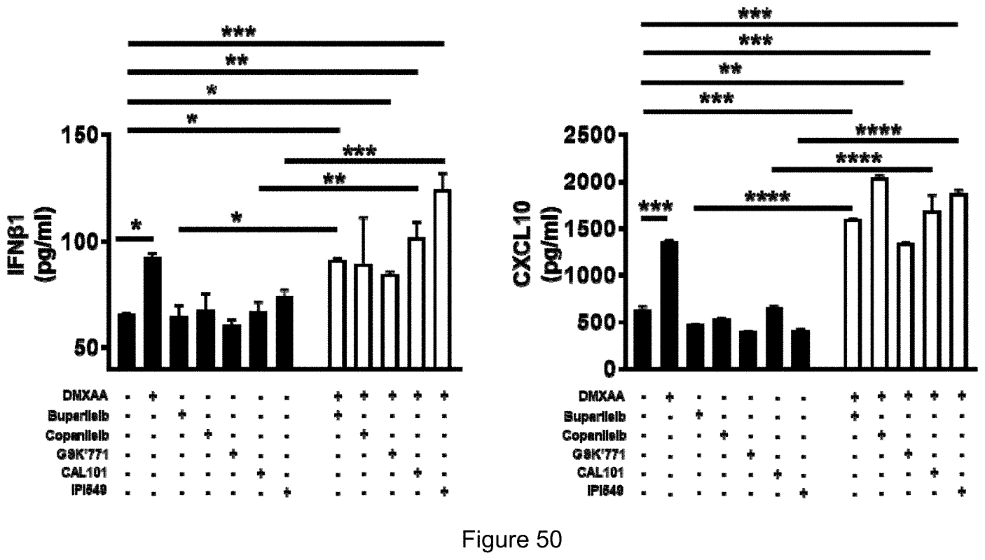

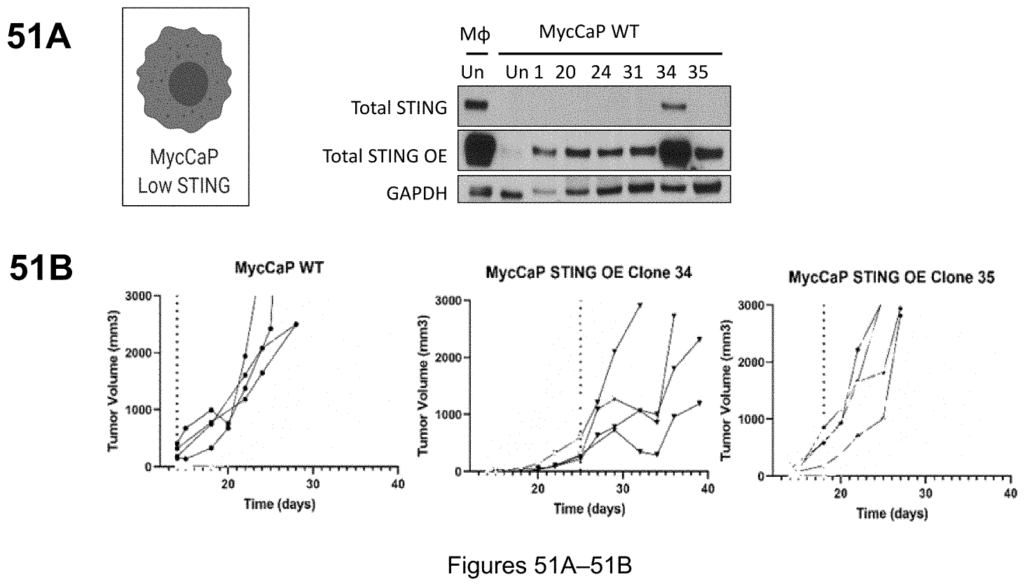

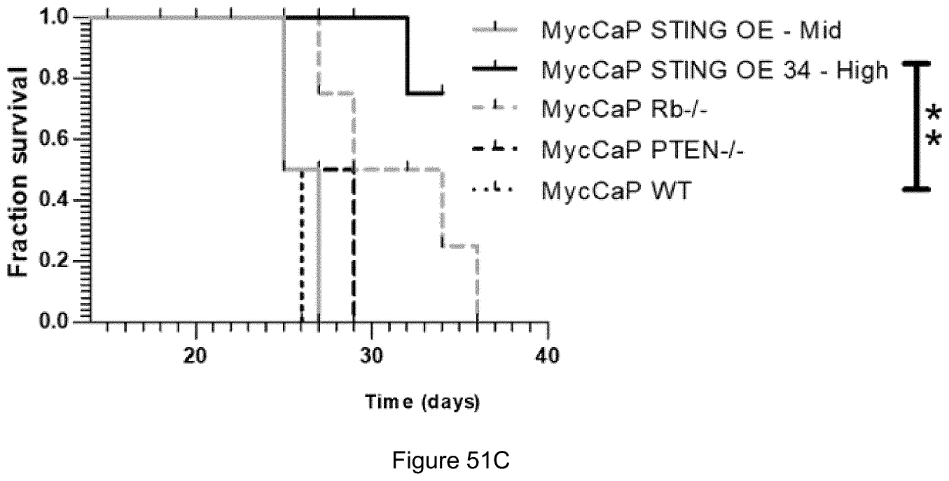

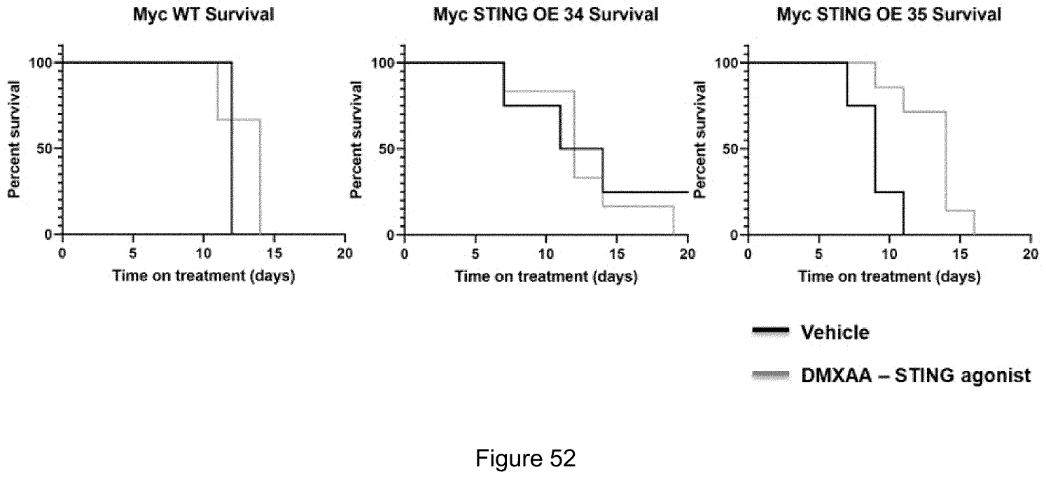

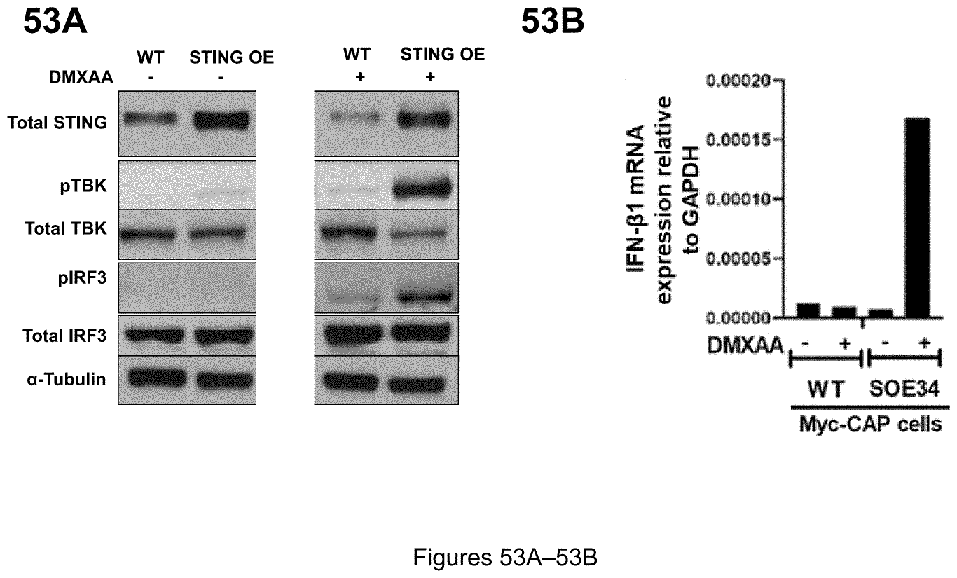

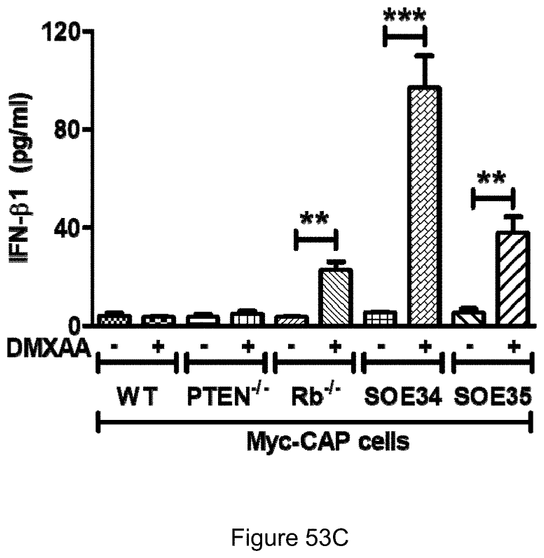

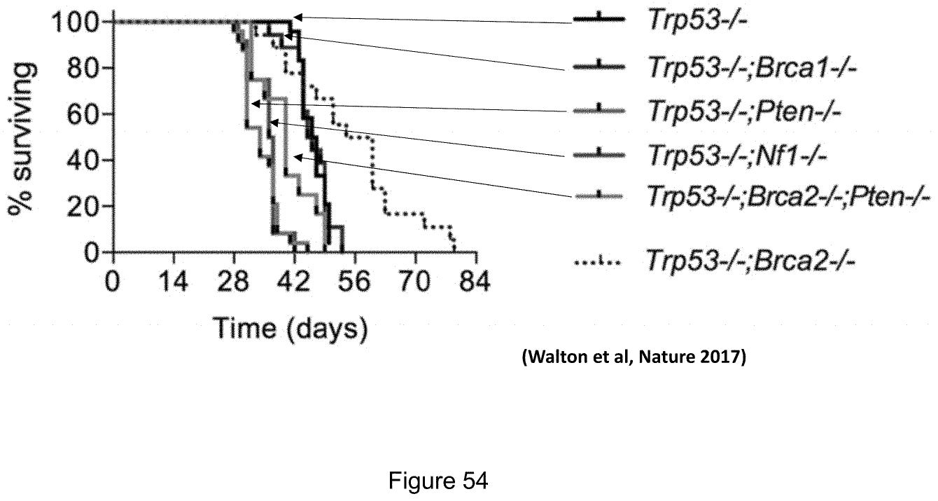

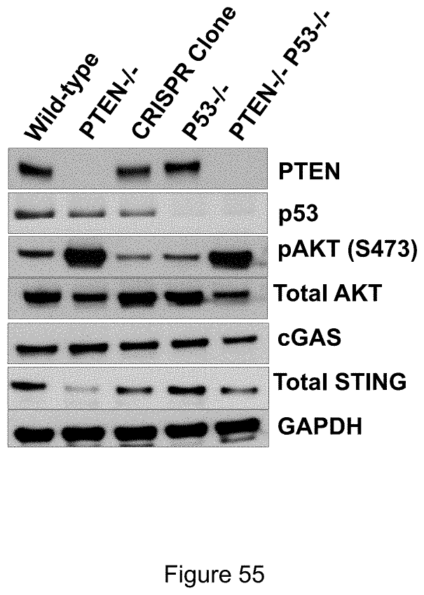

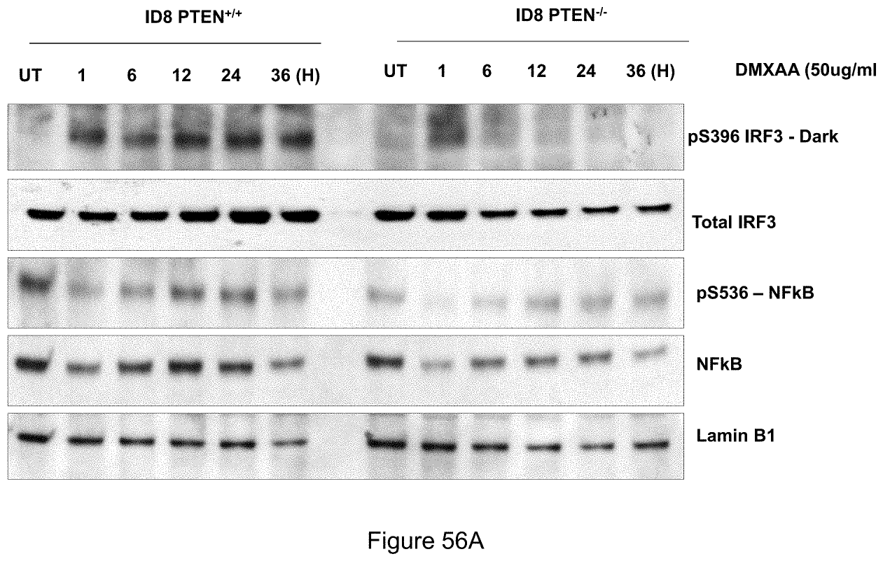

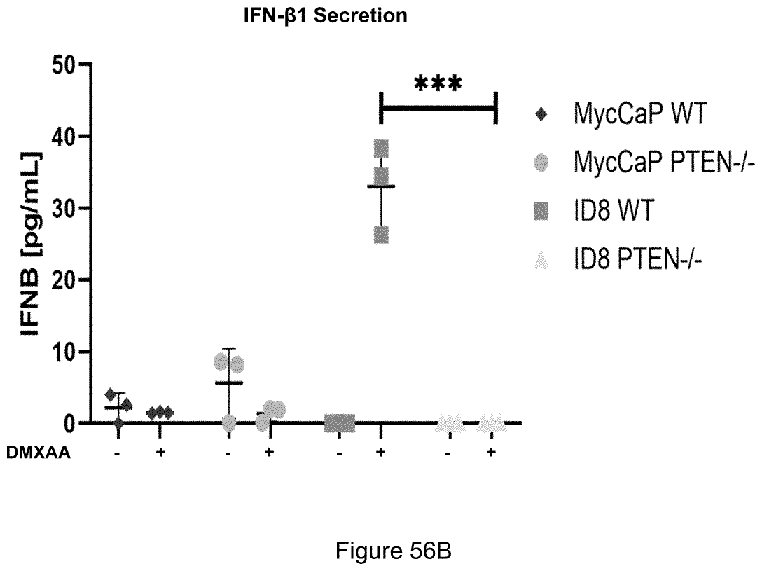

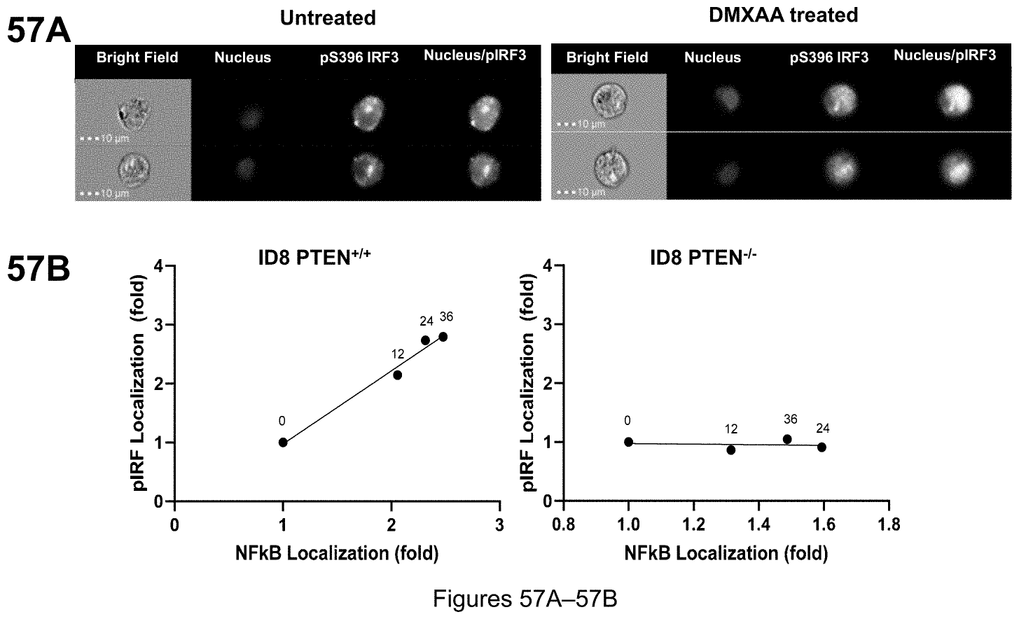

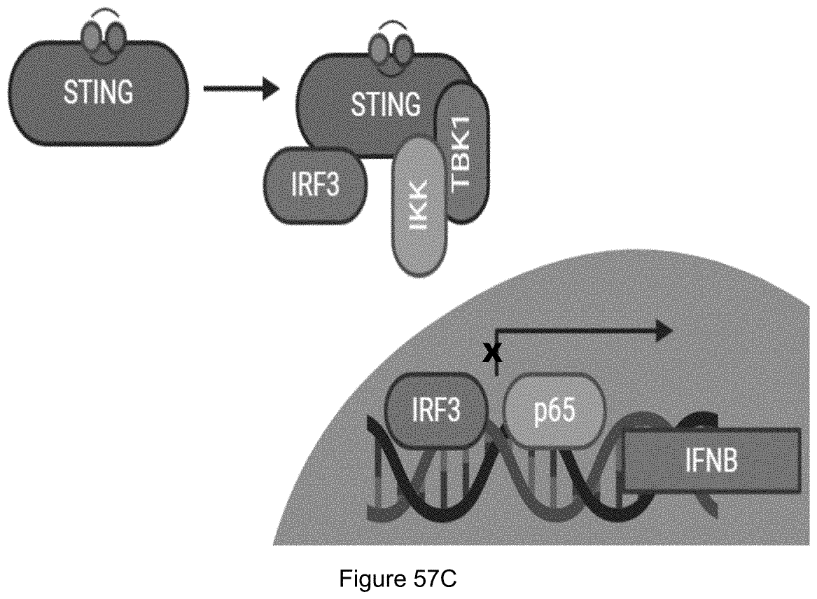

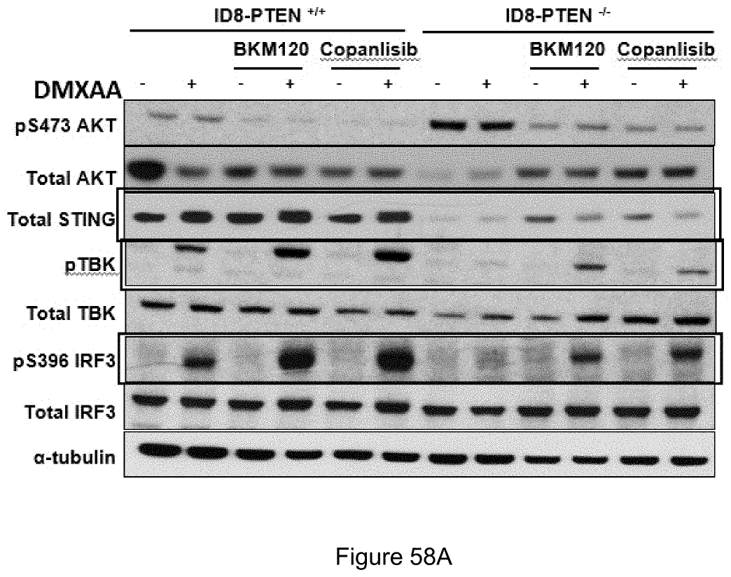

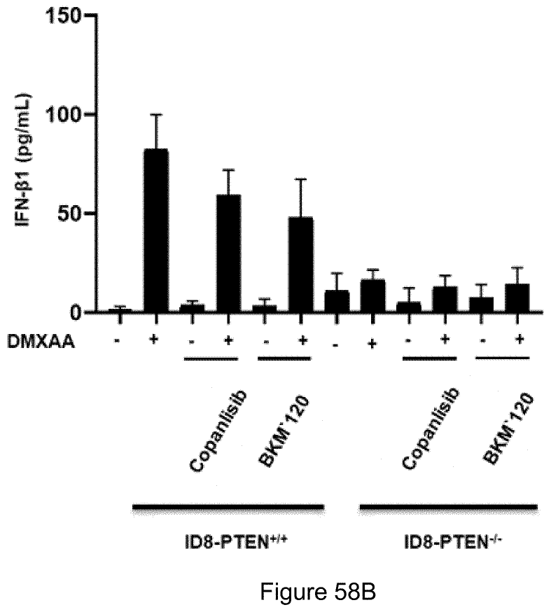

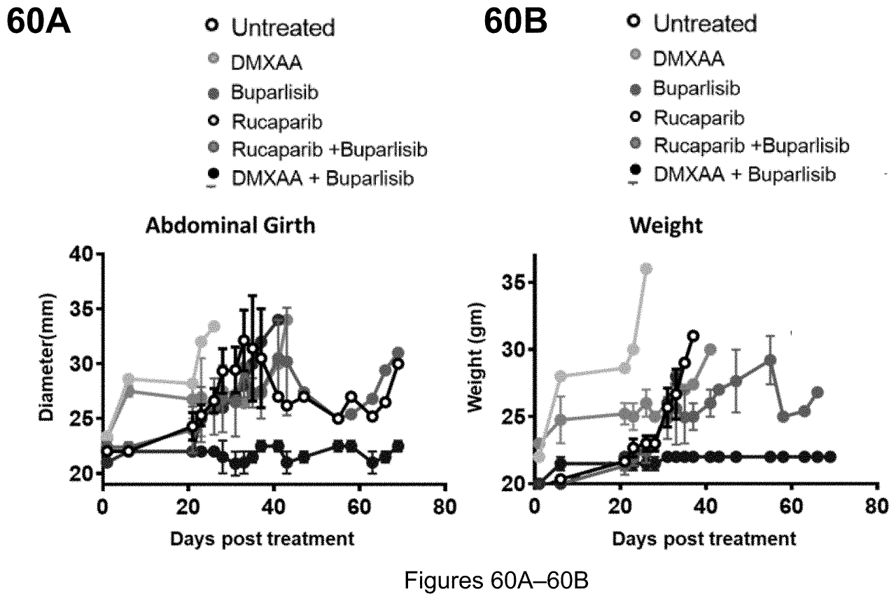

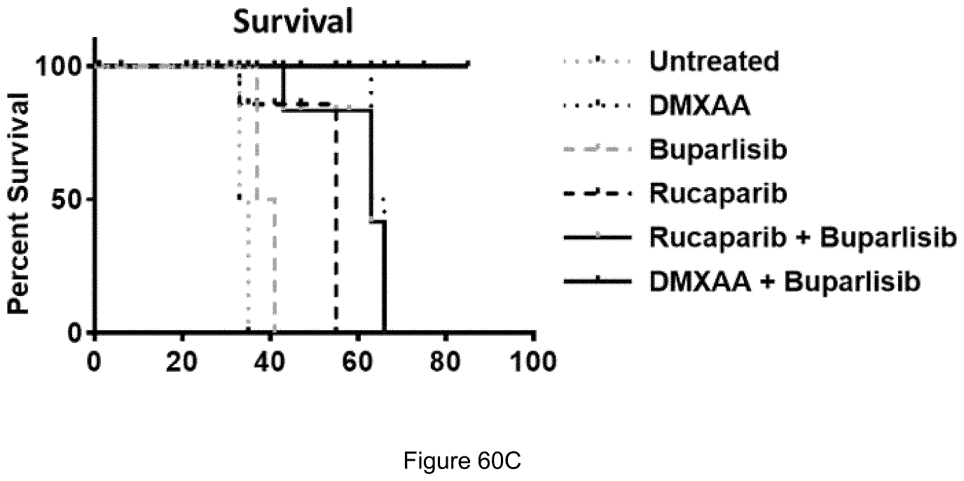

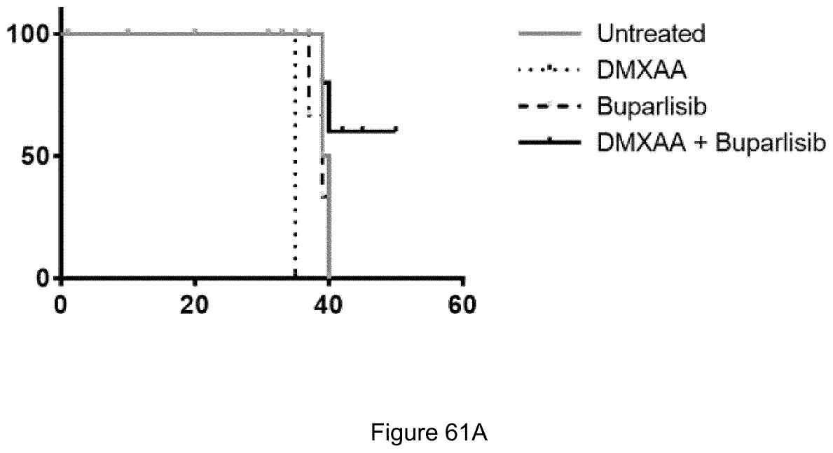

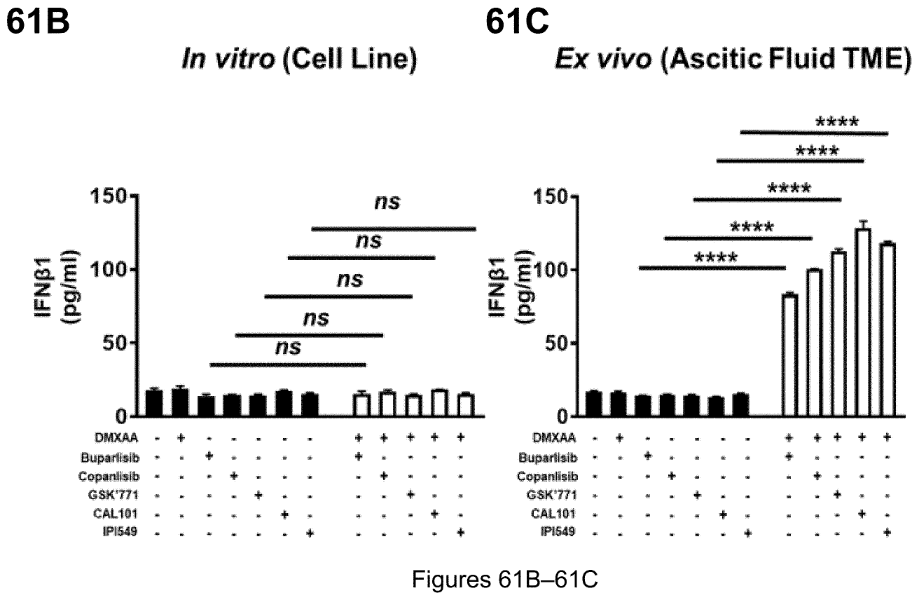

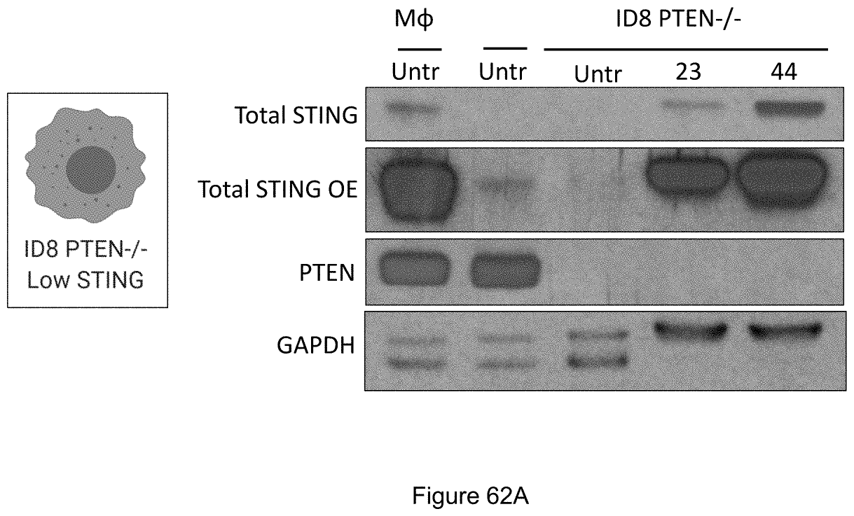

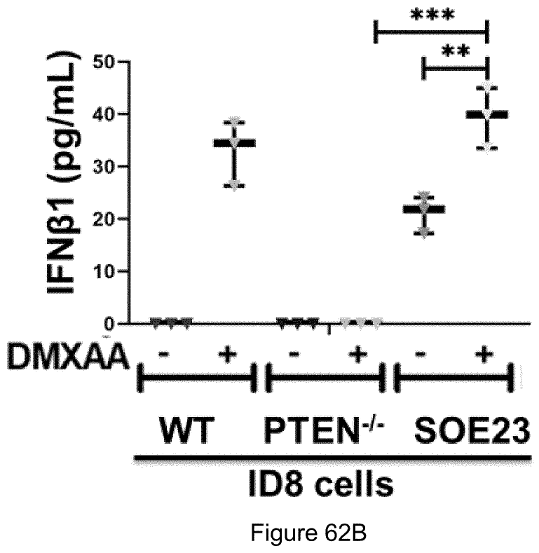

Data resulting from the above assays can be seen in FIGS. 33A-62B.

### Conclusions

PTEN loss is a resistance biomarker to PARPi/PI3Ki combination therapy in advanced prostate cancer. The presence of dsDNA associated with exosomes can be utilized as a surrogate immune biomarker of response to DNA damage-induced STING pathway activation, and predicts for responsiveness to PARPi/PI3Ki combination therapy. PTEN loss is a predictive biomarker to STING agonist/PI3Ki combination therapy in advanced prostate and ovarian cancer therapy. PTEN loss suppresses tumor cell intrinsic STING expression. STING agonist/PI3Ki combination therapy works via unopposed tumor cell extrinsic STING pathway activation within myeloid suppressive cells, and is not dependent on tumor cell intrinsic STING pathway activation.

### Example 4

### Mechanistic Studies and Therapeutic Approaches to Modulating the STING Pathway

### Summary

Overcoming limitations to therapeutic response in PTEN loss-of-function cancers. Immune checkpoint blockade (ICB) antibodies have had a major impact in a wide range of cancers. However, ICB therapeutic efficacy in prostate cancer (PC) is limited. Lack of T cell infiltration in melanoma has been associated with PTEN deletion, suggesting primary resistance mediated by this oncogenic event. Similarly, PTEN loss-of-function occurs in 50-75% of advanced prostate cancers and is associated with poor prognosis and treatment failure. These observations suggest a therapeutic opportunity to improve immunotherapy efficacy by overcoming the immunosuppressive tumor microenvironment (TME) in PTEN-mutant PC. Preclinical studies in c-myc-driven murine models have shown that PARP inhibitor (PARPi)-induced DNA damage within tumor cells, in combination with c-GAS de-repression within tumor-associated macrophages by PI3K inhibitors (PI3Ki), resulted in innate immune STING activation within the TME, and enhanced T cell infiltration and tumor regression in vivo. Strikingly, the anti-tumor mechanism of the PARPi/PI3Ki combination was primarily immune-mediated, as the effect was abrogated in immunodeficient mice. Critically, CRISPR/CAS9-mediated knockdown of PTEN in preclinical models resulted in de novo resistance of these tumors to DNA sensing STING pathway activation, and lack of an anti-tumor response elicited by PARPi/PI3Ki combination. The primary objectives of the present example are to elucidate the mechanism of de novo resistance of PTEN-deficient cancers to c-DNA sensing GAS/STING pathway within the TME, and to develop therapeutic immune-oncology (IO) strategies that enhance the immune TME and sensitize PTEN-deficient PCs to ICB.

Activating c-GAS/STING Signaling to Sensitize PTEN-deficient Prostate Cancer to ICB. Studies have demonstrated that isogenic PTEN loss leads to enhanced myeloid derived suppressor cell (MDSC) infiltration in c-Myc driven prostate cancers. Furthermore, PTEN-deficient Myc-CAP mice were differentially responsive to deliberate STING agonist treatment in vivo, relative to their PTEN-proficient counterparts. Tumor immune profiling studies demonstrated increased TAM and T cell activation following single-agent STING agonist treatment in the PTEN-deficient tumors, relative to their PTEN-proficient counterparts. In contrast, while concomitant ADT sensitized PTEN-proficient Myc-CAP tumor-bearing syngeneic mice to STING agonist treatment, this combination paradoxically abolished tumor control in syngeneic mice bearing PTEN-deficient Myc-CAP tumors, suggesting a detrimental impact of androgen-directed therapies on responsiveness of PTEN-deficient PC to STING agonist therapy. Furthermore, immunological quiescence was observed, with an absence of checkpoint upregulation within myeloid cells (TAMs, MDSCs and dendritic cells) of PTEN-deficient tumors, relative to PTEN-proficient tumors, following ADT treatment. Critically, prior studies have demonstrated that PI3Ki can de-repress c-GAS activity within TAMs, thereby priming these cells for innate immune activation with STING agonist therapy. Based on this data, the hypothesis is that PTEN deficiency drives an immunosuppressive TME via multiple mechanisms, which can be overcome by deliberate innate immune STING activation within TAMs.

The following studies are undertaken.

Elucidating the mechanism(s) by which PTEN-deficient PCs are de novo resistant to DNA-sensing STING pathway activation. Co-culture assays of PTEN-proficient PC cells with bone-marrow derived macrophages (BMDMs) have revealed that PARPi/PI3Ki combination therapy activates the c-GAS/STING pathway within BMDMs. This innate immune activation is mediated by double-stranded DNA (dsDNA)-containing microvesicles (MVs) released from PARPi-treated tumor cells coupled with c-GAS de-repression within PI3Ki-treated BMDMs, respectively. Critically, MVs released from PARPi-treated PTEN-deficient PC cells lack dsDNA, suggesting a block to dsDNA association to MVs. Furthermore, preliminary data demonstrates that PTEN loss also suppresses tumor cell intrinsic c-GAS/STING signaling. The mechanisms by which PTEN loss suppresses tumor cell-extrinsic and -intrinsic STING signaling are dissected. Collectively, these studies elucidate the mechanistic basis for the deleterious effects of PTEN loss on STING activation within the TME.

Dissecting the mechanistic basis for the differential therapeutic efficacy of STING agonists in PTEN-proficient vs. PTEN-deficient murine prostate cancer, with and without ADT.

Evaluating the differential therapeutic impact of direct STING agonists vs. PARP inhibitors, singly and in combination with PI3K inhibitors, on sensitization of PTEN-deficient murine PCs to ICB. Evaluating the anti-cancer mechanism and therapeutic efficacy of direct STING agonists, singly and in combination with PI3K inhibitors, on sensitization of PTEN-deficient murine PCs to ICB. Recent immune profiling studies have revealed a significant increase in MDSCs within the TME of PTEN-deficient vs. PTEN-proficient PCs. Rational immune-oncology (IO) combination strategies are used to re-invigorate STING within myeloid suppressive cells, thereby enhancing T cell infiltration within PTEN-deficient PCs. The central hypothesis is that direct STING activation within myeloid suppressive cells using a STING agonist (in combination with PI3Ki), but not indirect DNA damage-induced STING activation with PARPi/PI3Ki, overcomes the myeloid immunosuppressive TME and sensitizes PTEN-deficient PCs to ICB. The relative anti-tumor efficacy of STING agonists/PI3Ki vs. PARPi/PI3Ki combinations in PTEN-deficient murine models of PC, with or without ICB, is assessed. Taken together, the pre-clinical credentialing of these novel STING agonist-based IO combination strategies provides the mechanistic foundation for the next wave of clinical trials to eradicate PTEN-deficient PC.

Elucidating the tumor cell-intrinsic and -extrinsic mechanism(s) by which PTEN loss can drive immunosuppression within the TME. Analyzing changes in tumor immune microenvironment within metastatic biopsy samples from two investigator-initiated trials testing PD1-based IO combination therapies in mCRPC. An IO combination trial of rucaparib (PARPi) and nivolumab (PD-1 antibody) in mCRPC patients was conducted. Consistent with preclinical data, inadequate anti-tumor responses were observed. Metastatic biopsy samples from this trial are used to perform extensive functional and immunophenotypic analysis of the TME using buk/single-cell RNA sequencing, multiplex IHC, flow cytometry and transparent tissue tomography (3D-staining). These unbiased immune profiling approaches assess the relatively understudied myeloid compartment and allow us to compare patient-derived data to the preclinical data. Based on preclinical data described above, and an International ImmunoOncology Network collaboration with Bristol Myers Squibb® (BMS), a triple combination clinical trial of BMS-986301 (systemic STING agonist)/copanlisib/nivolumab is undertaken, with extensive correlative analyses on metastatic biopsy samples.

In summary, the project utilizes an integrative “co-clinical” approach to mechanistically elucidate and develop novel immune-based combination therapies to eradicate PTEN-deficient advanced PC.

### Introduction

There has been renewed interest in prostate cancer (PC) immunotherapy, partly based on the profound and durable clinical responses to immune checkpoint blockade (ICB) antibodies targeting CTLA-4 and PD-1/PD-L1 in other cancers. However, only approximately 10% of metastatic castrate-resistant prostate cancer (mCRPC) patients respond to ICB blockade. Emerging preclinical data suggests that targeting fundamental DNA repair and oncogenic signaling pathways could markedly increase responsiveness to immunotherapy. Activation of innate immunity in myeloid cells within the tumor microenvironment (TME) is a therapeutic approach to enhancing immune-responsiveness in PC.

In advanced PC, PTEN loss-of-function (LOF) is present in >50% of patients and is associated with poor prognosis and therapeutic outcomes. PTEN LOF leads to hyperactivation of the PI3K pathway, enhanced proliferation, reduced apoptosis, and increased genomic instability, the latter stemming from defects in homologous recombination (HR) repair. This causes the tumor cells to become reliant on poly (ADP-ribose) polymerase (PARP), and potentially to be vulnerable to PARP inhibitors (PARPi). However, clinical data has demonstrated that PTEN LOF is not a predictive biomarker for PARPi therapy. PTEN LOF has also been shown to drive an immunosuppressive TME and is associated with de novo and acquired resistance to ICB. Understanding the mechanism of PTEN LOF-induced immunosuppressive TME is therefore critical to developing novel therapeutic approaches to eradicate PTEN LOF tumors.

The cGAS/STING pathway is physiologically activated by cytosolic double-stranded DNA (dsDNA), which typically occurs in the context of viral infections, resulting in the generation of cytosolic cyclic dinucleotides generated by the cGAS (Cyclic GMP-AMP synthase) enzyme, downstream activation of the STING (Stimulator of INterferon Genes (STING)) pathway, and induction of Type I interferon (IFN) production. DNA damaging therapy can lead to STING pathway activation, and several studies are thus evaluating radiotherapy or PARP inhibitors with ICB in different solid tumor malignancies. However, recent clinical trial data has shown a lack of anti-tumor responses of PARPi/ICB combinations in mCRPC patients.

It has been demonstrated that STING activation within the myeloid compartment is critical to generating a robust immune infiltrate in the TME. Consistent with emerging clinical trial data, recent preclinical findings demonstrate that cytosolic DNA double-strand breaks (DSBs) elicited by PARPi within PTEN-proficient tumor cells, is insufficient to activate the innate immune DNA-sensing cGAS/STING pathway within myeloid suppressive cells of the TME. However, concomitant PI3Ki-mediated de-repression of cGAS enzymatic activity resulted in activation of PARPi-induced DNA sensing STING pathway activation within myeloid suppressive cells. This myeloid reprogramming was accompanied by enhanced T cell infiltration and tumor regression of PTEN-proficient, Myc-driven murine PC. The anti-tumor response elicited by PARPi/PI3Ki was abrogated in STING inhibitor-treated or immunodeficient mice. Critically, CRISPR/CAS9-mediated PTEN deletion in the Myc-model abrogated STING pathway activation and T cell infiltration within the TME, resulting in resistance to PARPi/PI3Ki treatment with or without ICB.

Data show that this resistance to DNA sensing STING pathway activation within PTEN-deficient PC arises via both tumor-cell intrinsic and extrinsic mechanisms. The mechanistic basis for this resistance can lead to alternative therapeutic strategies, such as direct STING agonist/PI3Ki-based combinations, that could sensitize PTEN-deficient PC to immunotherapy.

These studies involve “co-clinical” investigation of response/resistance mechanisms for immuno-oncology (IO) combinations in murine PC models in parallel with mCRPC patient biopsy samples from investigator-initiated clinical trials. Further, the focus is on evaluating anti-cancer mechanism and efficacy of STING agonist-based IO combination strategies in overcoming myeloid immunosuppression within the TME in PTEN-deficient preclinical PC models and mCRPC patients, thus providing the mechanistic foundation for the next wave of clinical trials. Lastly, the studies focus on using state-of-the-art technologies for integrative multidimensional analysis in mCRPC biopsy samples, and evaluating animal models of response and resistance.

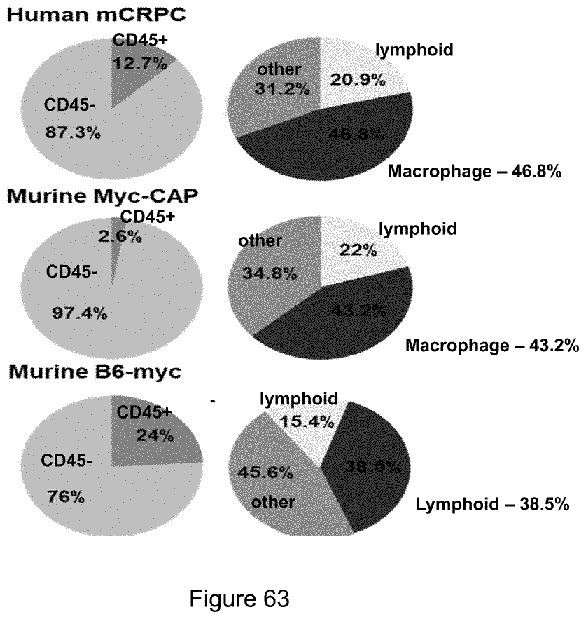

As a first step towards deconvoluting the metastatic tumor immune microenvironment in mCRPC, flow cytometry analysis of human mCRPC biopsy samples was performed. This revealed a paucity of CD45+ immune cells within the TME (FIG. 63). Furthermore, the small fraction of immune cells within the TME was predominantly immunosuppressive cells (˜70-80%), with tumor-associated macrophages (TAMs) comprising the highest frequency of CD45+ cells. Immune profiling of two Myc-driven murine prostate cancer syngeneic models, Myc-CAP and B6-myc, generated in FVB and C57BL6 genetic backgrounds, respectively, revealed a similar paucity of CD45+ immune cells (especially in the Myc-CAP tumors) and a predominance of myeloid suppressive cells and TAMs within the TME of both models.

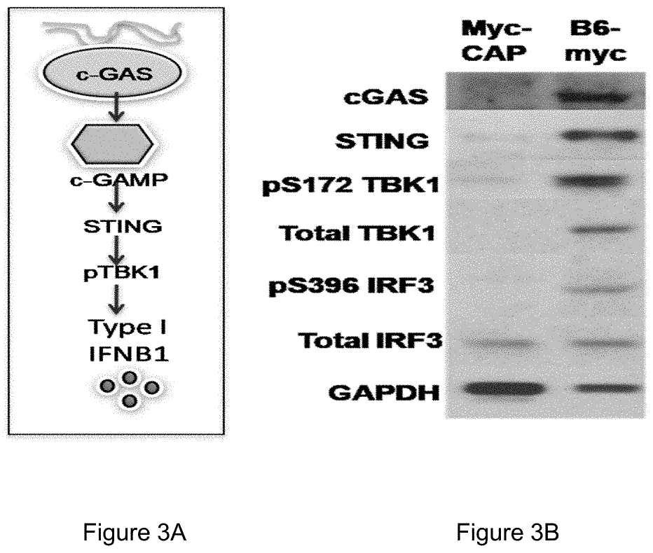

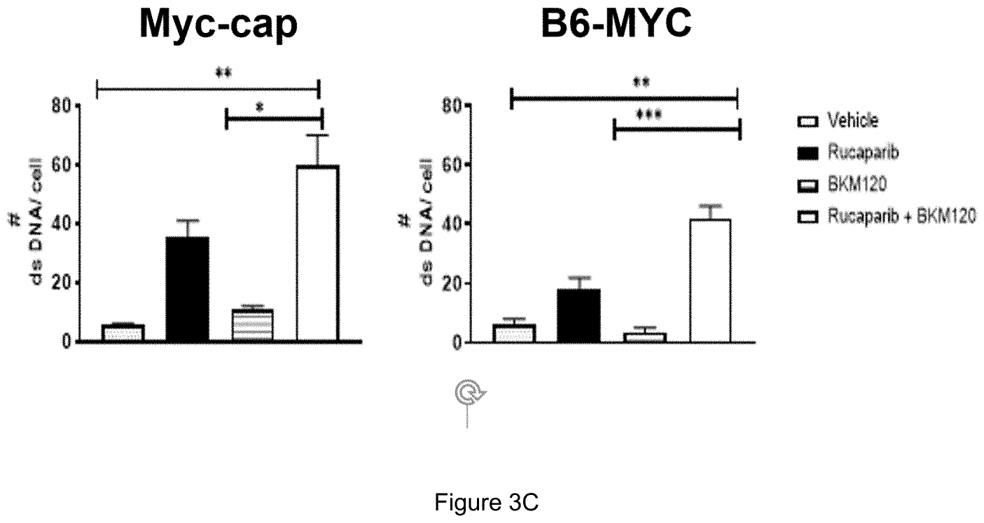

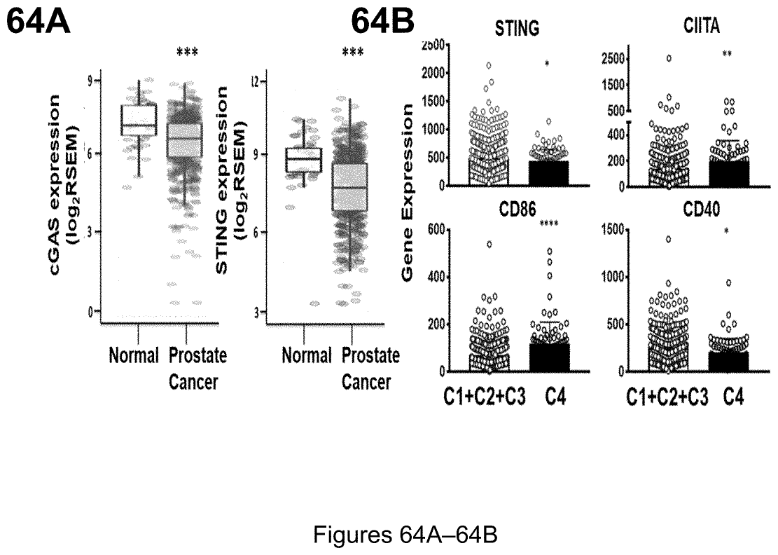

cGAS and STING expression in primary human PC samples was also assessed, and reduced expression was discovered relative to normal tissue counterparts (FIG. 64A). Furthermore, unsupervised hierarchical clustering of gene expression data, using previously published “low” and “high” Prostate Cancer Metastatic (PCM) signatures, revealed that poor prognosis patients had reduced STING and activated M1-like myeloid gene expression (FIG. 64B). These data suggest a correlation between STING expression, myeloid activation states, and clinical outcome. Notably, low and high cGAS/STING expression was also observed in the isogenic Myc-CAP and B6-myc cells, respectively (FIG. 3B), mimicking the STING gene expression patterns observed in low and high PCM risk groups within the TCGA. Based on these observations, it is envisioned that activation of cGAS/STING pathway within TAMs/myeloid suppressive cells in the TME enhances responsiveness to ICB in PTEN-deficient PC. In support of this concept, recent studies have shown that activation of the innate immune sensing cGAS/STING pathway results in activation of dendritic cells, strong downstream Type I IFN expression, recruitment of effector T cells into the TME, and a potent anti-tumor immune response in multiple preclinical models.

### Methods and Results

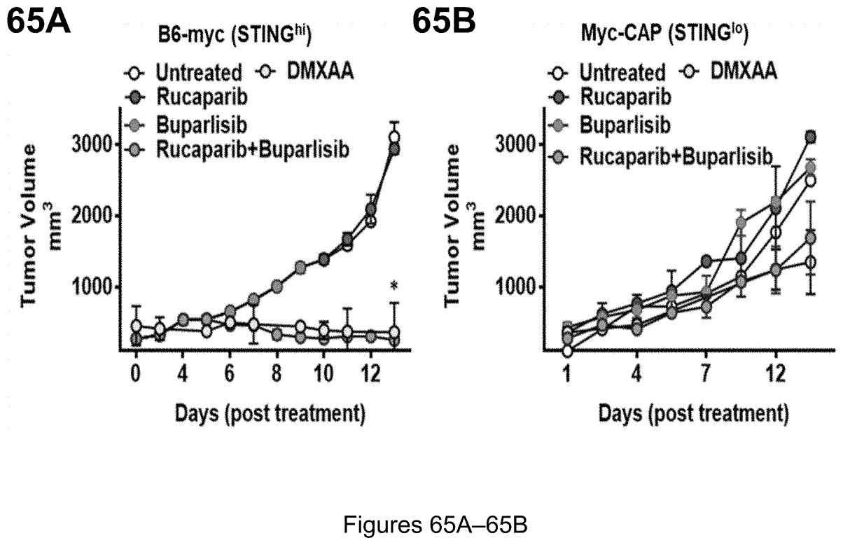

It has been previously demonstrated that the combination of PARP inhibitors (PARPi) and pan-PI3K (PI3Ki) inhibitors induce additive DNA damage and tumor regression in DNA damage repair (DDR) deficient breast, prostate, and ovarian cancers. In the B6-myc (STING^{hi}) DDR-proficient model, a significant increase in DNA DSB was observed following concomitant treatment with both rucaparib (PARPi) and buparlisib (PI3Ki), but not with corresponding single-agent treatments. While treatment of both B6-myc (STING^{hi}) and Myc-CAP (STING^{lo}) cell lines with PARPi/PI3Ki resulted in DNA damage in vitro, tumor regression in vivo was only observed in syngeneic B6-myc (STING) tumors (FIGS. 65A and 65B), suggesting that activation of both tumor cell-intrinsic and extrinsic STING signaling in response to DNA damage is critical for optimal anti-tumor responses.

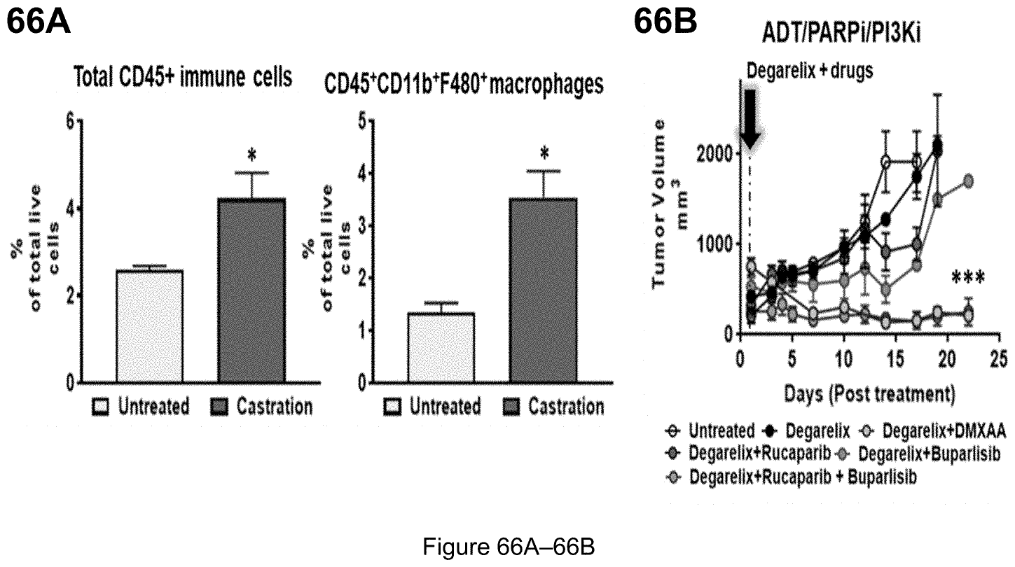

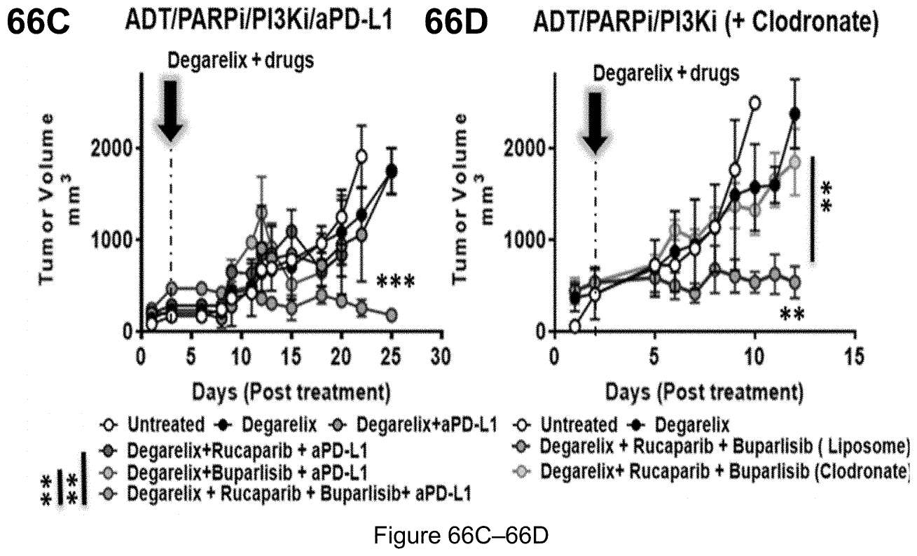

It is recognized that the aforementioned PARPi/PI3Ki studies were performed in a non-castrate model and that castration induces an immune infiltrate. Thus, the combination of PI3Ki/PARPi and castration in the Myc-CAP (STING^{lo}) tumor model was tested. Consistent with prior data, increased CD45+ and macrophage infiltration was observed following castration (FIG. 66A). However, castration alone or in combination with rucaparib or rucaparib/PD-L1 antibody was insufficient to control tumor growth. Strikingly, the combination of ADT plus PARPi/PI3Ki did result in tumor regression of Myc-CAP (STING^{lo}) tumors (FIGS. 66B-66C). Importantly, the anti-tumor response of ADT/PARPi/PI3Ki was abolished in immunodeficient NOD/SCID and nude mice, suggesting that this regimen induces tumor control via an immune-dependent mechanism. Furthermore, ADT/PARPi/PI3Ki triple combination led to increased macrophage infiltration/activation and CD4/CD8 T cell infiltration/activation within Myc-CAP tumors and this was enhanced by ICB. Gene expression (qRT-PCR) analysis of tumor extracts from ADT/PARPi/PI3Ki-treated tumors demonstrated increased Tbet and IL12 expression, and flow cytometry revealed decreased Arginase expression within macrophages, thus demonstrating that the triple combination enhanced Th1 cell and M1 macrophage polarization. To test the hypothesis that macrophage activation is responsible for the observed anti-tumor immune response, Myc-CAP syngeneic tumor-bearing mice were treated with concomitant clodronate, which systemically depletes macrophages, in combination with ADT/PARPi/PI3Ki. Critically, it was observed that clodronate abolishes the tumor regression observed with ADT/PARPi/PI3Ki treatment (FIG. 66D). Taken together, these data demonstrate that castration/PARPi/PI3Ki combination induces a macrophage-mediated, innate immune response, resulting in tumor clearance.

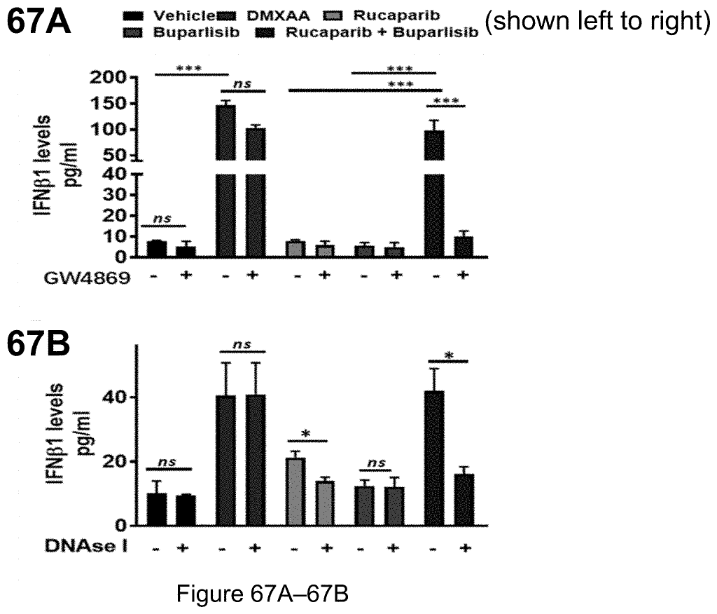

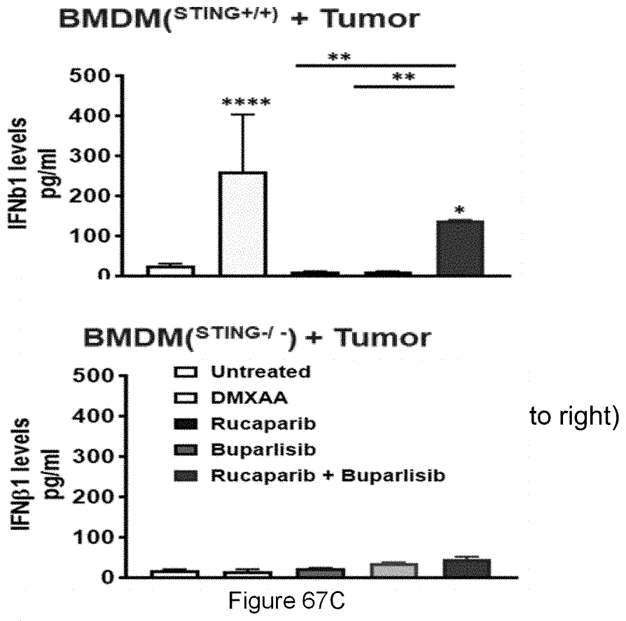

To elucidate the anti-cancer innate immune mechanism elicited by PARPi/PI3Ki combination in Myc-CAP (STING^{lo}) tumors, Myc-CAP cells were treated in vitro with PARPi, singly and in combination with PI3Ki, at their respective IC90 concentrations. Interestingly, it was observed that PARPi caused an increase in DNA DSBs, as measured by number of g-H2AX foci, which was not enhanced by the addition of PI3Ki. These data suggest that concomitant PI3Ki treatment activates macrophage-mediated innate immunity by a DNA-damage independent mechanism in Myc-CAP (STING^{lo}) tumors. To determine whether PARPi/PI3Ki-induced DNA damage activates the c-GAS/STING pathway within the TME, an ex vivo reconstitution assay was performed using single-cell suspensions derived from Myc-CAP (STING^{lo}) cells treated with exogenous PARPi, singly and in combination with PI3K. An increase in Type 1 IFN production (read-out of STING pathway activation) was observed within the supernatants following ex vivo PARPi/PI3Ki combination treatment, and not observed with either single agent (FIG. 67A). To test the hypothesis that microvesicle (MV) transport of DNA DSBs from tumor cells within the TME results in c-GAS/STING activation, ex vivo assays were conducted using single-cell suspensions of Myc-CAP tumors treated with PARPi/PI3Ki in the presence or absence of GW4869, an inhibitor of MV biogenesis and release. Significant down-regulation of Type I IFN production was observed following concomitant PARPi/PI3Ki and GW4869 treatment in vitro (FIG. 67A), demonstrating that c-GAS/STING pathway activation occurs via MV-associated DNA DSBs released from tumor cells.

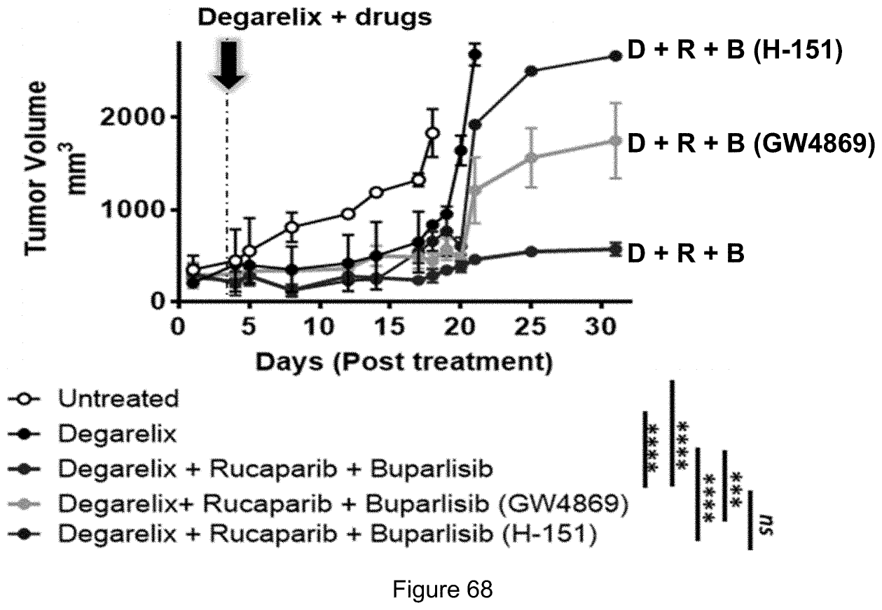

To specifically test the role of DNA DSB-associated MVs released from tumor cells on c-GAS/STING pathway activation of macrophages, co-culture of bone marrow derived macrophages (BMDMs) with DNase I-treated MVs was performed, and a striking decrease of IFNB1 release was observed relative to untreated MVs (FIG. 67B). Importantly, co-culture of Myc-CAP cells with BMDMs treated with PARPi/PI3Ki elicited type I interferon production within the supernatant. This effect was abrogated when Myc-CAP cells were co-cultured with STING^{-/-}BMDMs (FIG. 67C). In vivo concomitant treatment with GW4869 or H-151 (STING inhibitor) abrogated the anti-tumor response elicited by PARPi/PI3Ki treatment in Myc-CAP (STING^{lo}) syngeneic mice (FIG. 68). Collectively, these studies revealed that PARPi/PI3Ki increased tumor-cell extrinsic c-GAS/STING pathway activation within macrophages via MVs carrying dsDNA cargo released from drug-treated tumor cells.

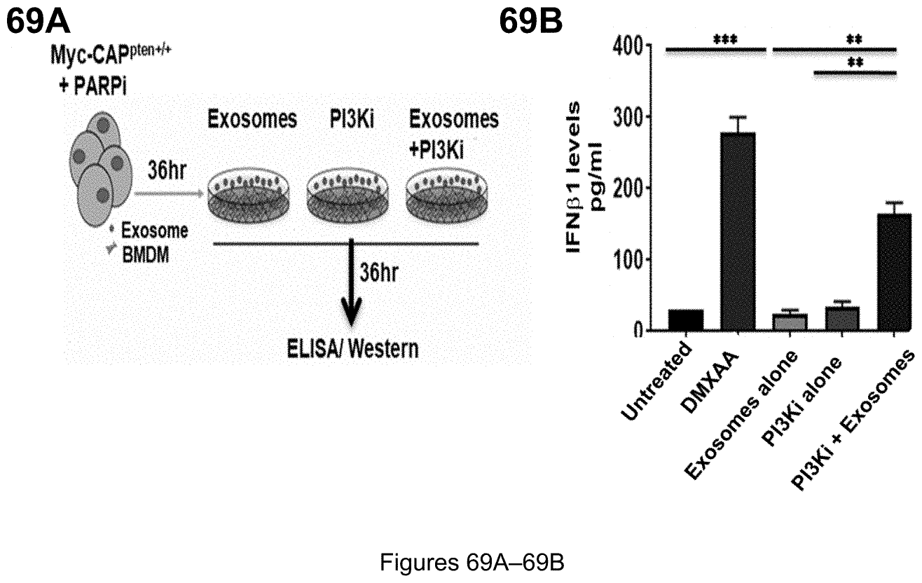

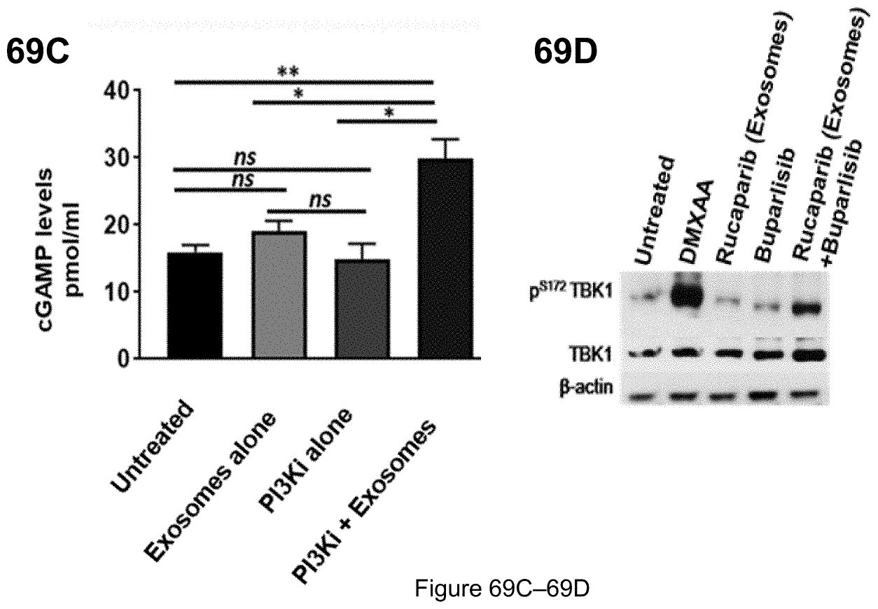

Recent work has shown that AKT can phosphorylate c-GAS at Ser-291, leading to post-translational suppression of its enzymatic activity. It was therefore hypothesized that PI3Ki treatment can de-repress c-GAS enzymatic activity resulting in increased production of STING ligand 2′3′-cGAMP. The impact of microvesicles or exosomes (referred to herein interchangeably as MVs, microvesicles, or exosomes) collected from PARPi-treated Myc-CAP cells was assessed. BMDMs were treated with MVs alone, PI3Ki alone, or the combination of MVs/PI3Ki. While MVs alone or PI3Ki alone were insufficient to activate the c-GAS/STING pathway within macrophages, the combination of MVs/PI3Ki resulted in potent type I IFN production, similar to what was achieved with direct STING agonists (FIGS. 69A-69B). To specifically assess why PARPi-induced DNA DSBs associated with exosomes was necessary but not sufficient to activate c-GAS/STING pathway within macrophages, and whether PI3Ki relieved c-GAS post-translational suppression, levels of intra-cellular cGAMP was measured within macrophages by ELISA. A significant induction of cGAMP was observed only in the presence of both MVs and PI3Ki (FIG. 69C), resulting in increased c-GAS/STING pathway activation with macrophages (FIG. 69D). Taken together, these results demonstrate that the presence of PARPi-induced DNA DSB immunogen is insufficient to activate c-GAS/STING pathway within macrophages, and concomitant PI3Ki-induced cGAS de-repression within myeloid suppressive cells is required for optimal activation and re-programming of macrophages from M2 to M1 within the TME.

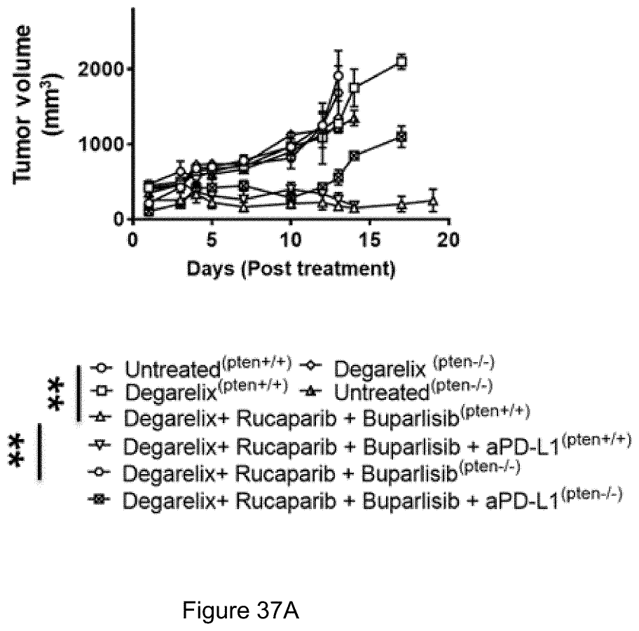

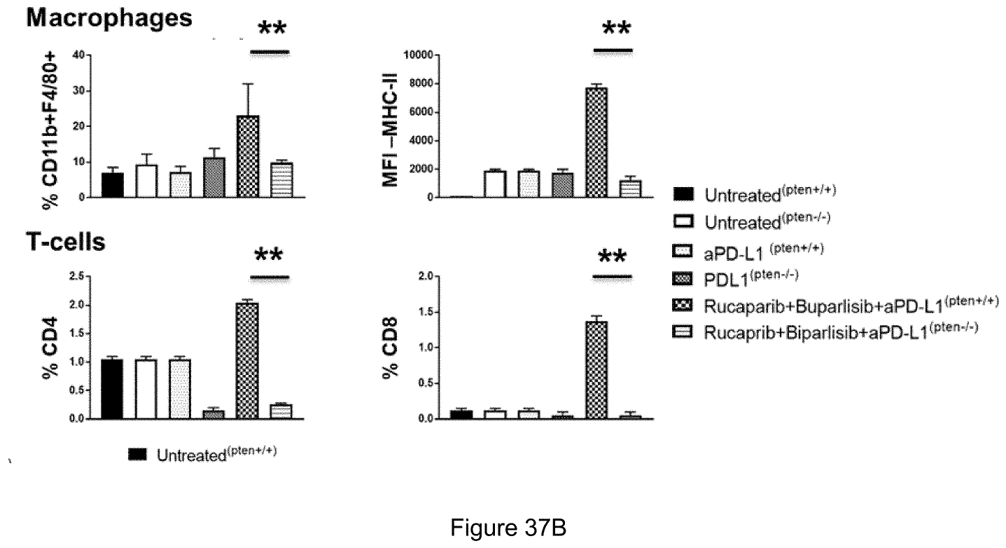

Because of the relevance of PTEN deletion in PC, a Myc-CAP-PTEN deficient model was generated via CRISPR/CAS9-mediated deletion of PTEN. It was observed that these PTEN-deficient tumors have increased infiltration of granulocytic-myeloid derived suppressor cells (Gr-MDSCs), and decreased infiltration of CD4, CD8, and dendritic cells within the TME, relative to their PTEN-proficient counterparts. Similarly, PTEN-deficient primary and metastatic prostate cancer samples from the TCGA and SU2C had a significant enrichment of MDSC-high tumors. The therapeutic impact of the PARPi/PI3Ki/ADT combination was evaluated in a Myc-CAP-PTEN deficient model. Unlike Myc-CAP PTEN-proficient tumors, Myc-CAP-PTEN deficient tumors were resistant to this combination treatment, with or without ICB (FIG. 37A) and did not show enhanced tumor macrophage or CD8/CD4 T cell infiltration (FIG. 37B). Thus the mechanistic basis being pursued is that PTEN deficient-prostate cancers are de novo resistant to immunotherapy through dysregulation of tumor cell intrinsic and extrinsic DNA-sensing STING pathway activation within the TME, and this can be overcome by STING agonist-based IO combinations.

Elucidating the Mechanism(s) by which PTEN-Deficient Prostate Cancers are De Novo Resistant to DNA-Sensing STING Pathway Activation.

Elucidating the tumor-cell extrinsic mechanisms by which PTEN-deficient prostate cancers are de novo resistant to DNA-damage induced STING pathway activation.

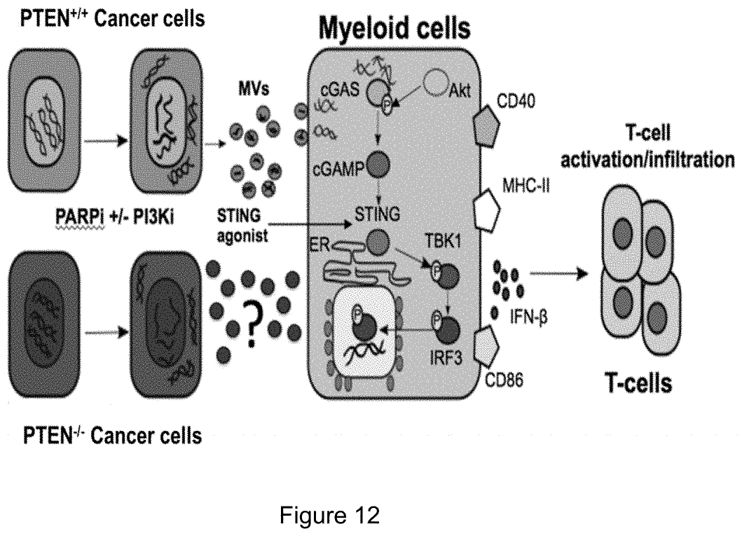

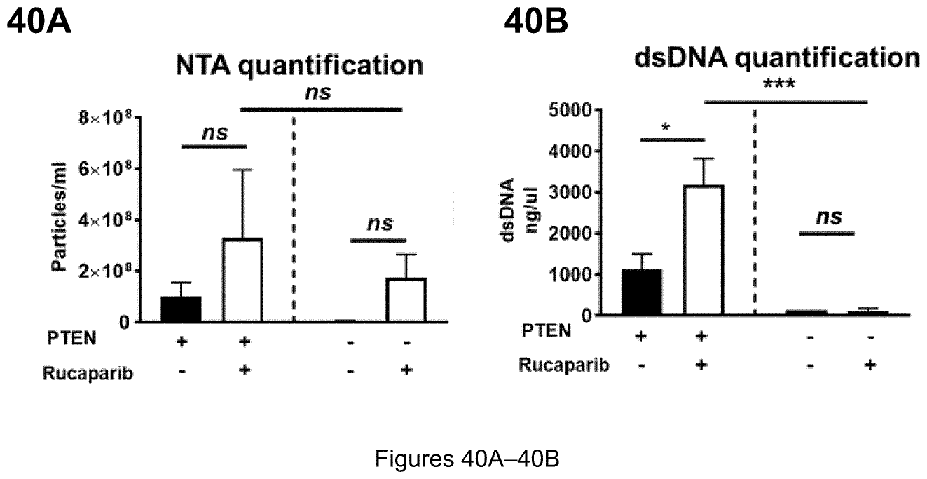

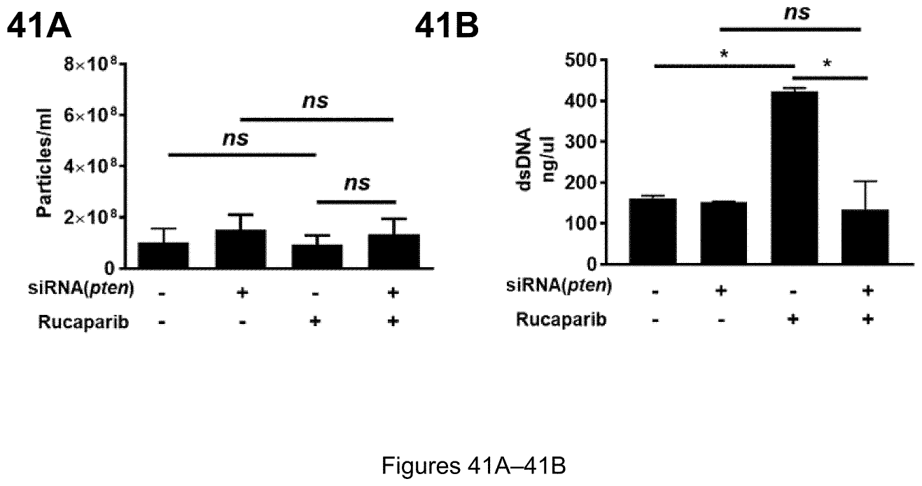

The approaches herein rely on the premise that PTEN-deficient PCs are resistant to STING pathway activation within the TME, due to a defect in the association of DNA DSBs with exosomes/microvesicles (MVs) released from tumor cells. Deletion of PTEN impaired STING activation by MVs derived from PARPi/PI3Ki-treated cells. Similar results were obtained in ex vivo studies utilizing prostate tumors from PTEN-deficient GEMMS (data not shown). While there was no difference in MV quantity released from PARPi-treated PTEN-proficient vs. CRISPR/CAS9-mediated PTEN-deficient Myc-CAP (STING^{lo}) cells (FIG. 40A), MVs released from PTEN-deficient PC cells had a significant decrease in associated DNA DSBs (FIG. 40B). Furthermore, transient knockdown of PTEN in Myc-CAP cells also resulted in a loss of DNA DSB association with MVs (without a change in quantity released) following PARPi treatment (FIG. 41B). A model summarizing the above studies has previously been presented in FIG. 12.

To elucidate the mechanistic basis for tumor cell-extrinsic resistance to STING pathway activation within PTEN-deficient PC, isogenic Myc-CAP (STING^{lo}) and B6-myc (STING^{hi}) cells+/−CRISPR/CAS9-mediated PTEN deletion are utilized. MVs are purified by ultracentrifugation from conditioned-media of these drug-treated cell lines, as well as wild-type/PTEN-deficient human PC cell lines. A DNA DSB association defect within MVs released from PARPi-treated PTEN-deficient cancer cells is first confirmed by performing unbiased comprehensive exosome subset and cargo analysis. Then PTEN catalytic activity is pharmacologically inhibited using bpV(HOpic), a PTEN protein phosphatase inhibitor, and MVs purification and subset/cargo analysis is performed to determine whether PTEN lipid vs. protein phosphatase activity is required. Using CRISPR-CAS9-based genome editing, endogenous mutants of PTEN are engineered that lack either protein or lipid phosphatase activity, to determine whether PTEN catalytic or scaffolding function is necessary for DNA DSB association with MVs. Given the importance of phosphoinositide signaling in MV/exosome biogenesis and protein sorting into multivesicular bodies, PTEN lipid phosphatase activity can be critical for DNA DSB association with exosomes. Confocal microscopy experiments are used to delineate the precise localization of DNA DSB either within or on the surface of exosomes isolated from drug-treated tumor cells. The differential impact of different PTEN mutants on biogenesis of small MVs (50-200 nm) vs large MVs or oncosomes (1 μm) is evaluated, the latter group budding directly from the plasma membrane. To confirm that PTEN loss alters phospholipid distribution and net charge on the membrane surface resulting in abrogation of electrostatic interactions with DNA DSBs, the impact of PTEN loss on phospholipid composition is examined in isogenic murine PC cells, using electrospray ionization-mass spectrometry. MVs and oncosomes are purified from PTEN-proficient and PTEN-deficient human mCRPC organoids, and comprehensive cargo analysis, including determining DNA DSB content, is performed.

Alternative approaches. It is conceivable that the PARPi has a direct DNA damage-independent effect on transcriptional reprogramming of macrophages. RNA-seq and Type I interferon ex vivo ELISA assays are performed in macrophages that are directly treated with PARPi and/or PI3Ki. To determine the generalizability of the mechanisms of DNA sensing c-GAS/STING pathway activation within macrophages in the TME, ionizing radiation and/or platinum chemotherapy is also used as alternative strategies to induce DNA damage in the PTEN-proficient vs. PTEN-deficient murine PC models. While a significant abrogation of PARPi/PI3Ki-mediated anti-tumor responses in Myc-CAP (STING^{lo}) syngeneic mice with GW4869 was observed, it did not completely rescue tumor growth to levels seen in the presence of concomitant STING inhibitor treatment—it is possible that there are additional exosome-independent mechanism(s) by which this drug combination can activate the c-GAS/STING pathway. To explore these mechanism(s), deep immune profiling of the TME is performed within these tumors following PARPi/PI3Ki combination treatment, using bulk/single-cell RNA-seq and flow cytometry/multiplex IHC.

Elucidating the tumor-cell intrinsic mechanisms by which PTEN-deficient PCs are de novo resistant to STING pathway activation.

PARPi/PI3Ki combination inhibits B6-myc (STING^{hi}), but not Myc-cap (STING^{lo}) syngeneic prostate tumors (FIG. 65). It has also been demonstrated that human primary PCs have reduced STING expression, which correlates with poor prognosis in PC (FIG. 64). These studies are designed to confirm that PTEN loss results in tumor cell-intrinsic suppression of STING expression and downstream Type 1 IFN signaling, which in turn contributes to ICB resistance.

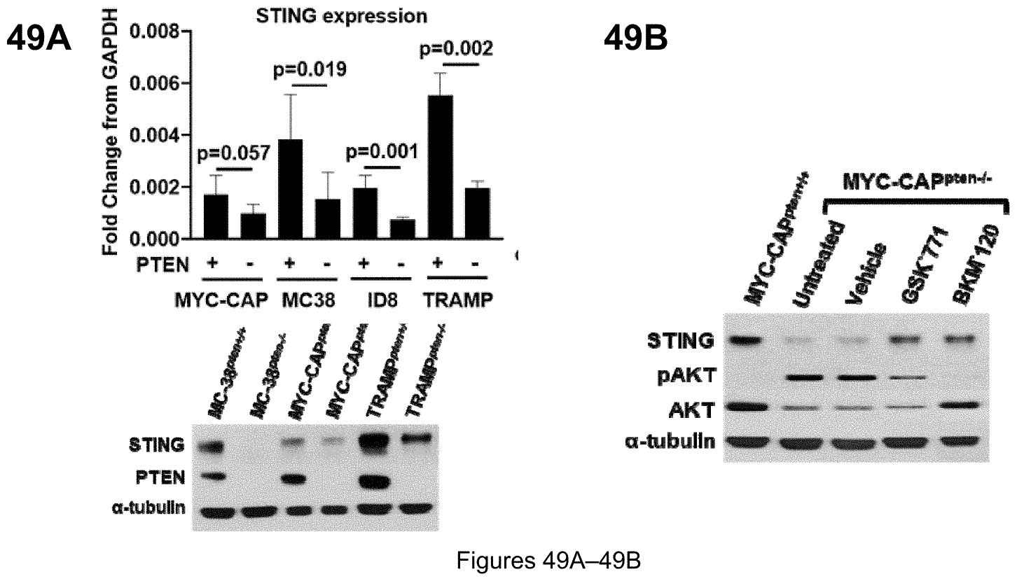

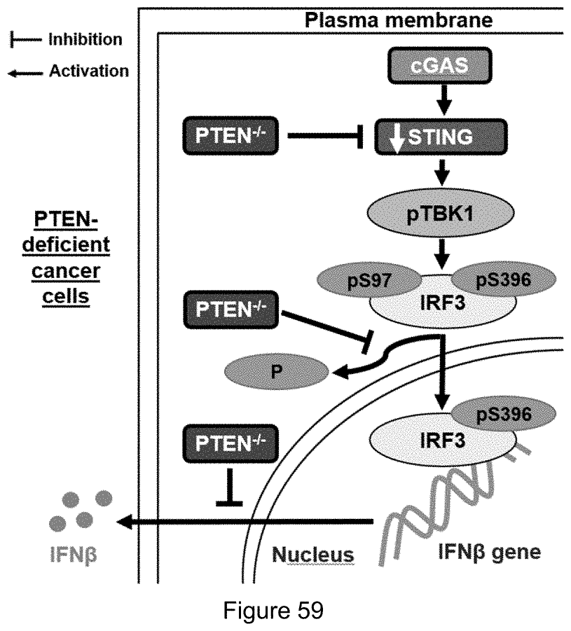

Using the aforementioned isogenic cell lines, it was observed that PTEN loss decreases STING mRNA and protein expression across multiple cancer cell lines (FIG. 49A-49B). The downstream readout of canonical c-GAS/STING pathway activation is IRF3 translocation to the nucleus and type I interferon production. Recent data has demonstrated that PTEN regulates anti-viral immunity by enhancing IRF3 translocation to the nucleus, via Ser-97 dephosphorylation on IRF3. Consistent with this observation, studies in murine PTEN-deficient cell lines show cytosolic sequestration of p-IRF3. In addition, STING agonist treatment of PTEN-deficient cancer cells resulted in an absence of Type I interferon production. These data suggest that PTEN LOF negatively regulates the c-GAS/STING/p-TBK1/p-IRF3 axis at multiple vertical nodal points, resulting in suppression of Type I IFN production from the tumor cell (FIG. 59).

To test the relative impact of PTEN LOF on suppression at different nodal points along the c-GAS/STING/p-TBK1/p-IRF3 axis, doxycycline-inducible STING overexpression is engineered in Myc-CAP (STING^{lo}) cells, and the ability of STING agonists to rescue type I interferon production in vitro is tested. Furthermore, CRISPR-CAS9 is utilized to engineer IRF3-S97A mutations in Myc-CAP cells (STING^{lo}) and test whether IRF3-S97A, which would be expected to translocate to the nucleus following c-GAS/STING activation, can bypass the PTEN loss-mediated block to STING agonist-induced Type I IFN production. In the event that STING overexpression or IRF3-597A mutation alone is insufficient to rescue Type I IFN production, a compound cell line mutant can be engineered that contains both the doxycycline-inducible STING and the IRF3-S97A mutation, and the mutant can be assessed for rescue of Type I IFN production following doxycycline/STING agonist treatment. In addition, STING agonist/PI3Ki combinations are tested in order to determine the relative impact of AKT-mediated c-GAS suppression on Type I IFN production from PTEN-deficient tumor cells. Collectively, these studies shed light on relative contributions of c-GAS post-translational suppression, decreased STING expression and IRF3 cytoplasmic sequestration on type I IFN signaling within PTEN-deficient PC cells.

Since PTEN LOF suppresses STING gene expression, and PI3K signaling is involved in epigenetic silencing through modifications in histones or DNA, it was hypothesized that PI3Ki enhance c-GAS/STING pathway activation within the TME, in part, via restoration of tumor cell intrinsic STING expression. Data indicates that PI3Ki can indeed partially rescue STING expression (FIG. 49C). The molecular mechanisms involved in PTEN LOF-induced epigenetic silencing of STING promoter are dissected. Specifically, a screen is performed with DNA/histone methylation or acetylation inhibitors for their ability to restore STING expression and cGAS/STING signaling in B6-myc-PTEN deficient cells and human PTEN-deficient PC cells. Global methylome profiling is performed on PTEN-proficient and PTEN-deficient cells to look for differential methylation of the STING promoter. To explore genes/transcription factors with a potential role in STING gene regulation, a promoter pull down assay for STING is performed, using nuclear extracts derived from B6-myc (STING) isogenic cells (−/+PTEN), and a biotinylated STING promoter, followed by mass spectrometry analysis of promoter-bound proteins. In addition, it was recently shown that partial rescue of Type I IFN gene expression by PI3Ki occurs despite cytoplasmic sequestration of IRF3. As an exploratory step to identify new transcription factors binding to the interferon promoter following STING agonist/PI3Ki treatment, a similar promoter pull down assay is performed for IFNβ1, followed by mass spectrometry analysis of promoter bound proteins. Taken together, this convergent experimental approach sheds light on the mechanism(s) for c-GAS/STING/type I IFN pathway regulation in PTEN-deficient PC cells.

Alternative approaches. Given the mutational heterogeneity across PC cell lines, there can be cell-type specific differences in PTEN-loss induced downregulation of STING expression. A more comprehensive analysis can be performed using both human PC cell lines as well as non-prostate cancer cell lines, to confirm the generalizability of these observations. As noted above, PTEN LOF can regulate alternative signaling pathways that suppress tumor cell intrinsic type I IFN gene transcription. Isogenic B6-myc cells (−/+PTEN) can be utilized to explore the differential effects of PTEN LOF on the status of the canonical and non-canonical NF-κB signaling, given recent studies showing opposing effects of canonical and non-canonical NF-κB signaling on IRF3-mediated Type I IFN production. Occupancy of p65 on the IFNβ1 promoter can also be measured, and multiplex transcription profiling can be performed using the Signosis array promoter pull-down assay, accompanied by mass spectrometry, to discover alternative transcription factors that can restore Type I IFN production following STING agonist/PI3Ki treatment in PTEN-deficient cancer cells.

Biostatistical analysis. This evaluation involves statistical analyses of continuous assays in cell lines. Descriptive statistics are used to summarize data for experimental conditions. T-tests or linear regression models with treatment or mutation group as predictor are used to compare outcomes across experimental conditions. Each experimental condition for each cell line is replicated n=6 times. This repetition provides 80% power and two-sided 5% Type I error to detect standardized effect size of Δ=1.8 based on a two-sample t-test. While these effect sizes are large, only substantial differences in cell line experiments prompt moving ahead with additional experiments. In some data, differences at least as large have been observed (e.g. FIG. 40).

Evaluating the Differential Therapeutic Impact of Direct STING Agonists vs. PARP Inhibitors, Singly and in Combination with PI3K Inhibitors, on Sensitization of PTEN-Deficient Murine PCs to ICB.

Elucidating the relative anti-tumor efficacy for STING agonists/PI3Ki vs. PARPi/PI3Ki combinations in P TEN-deficient murine models of PC.

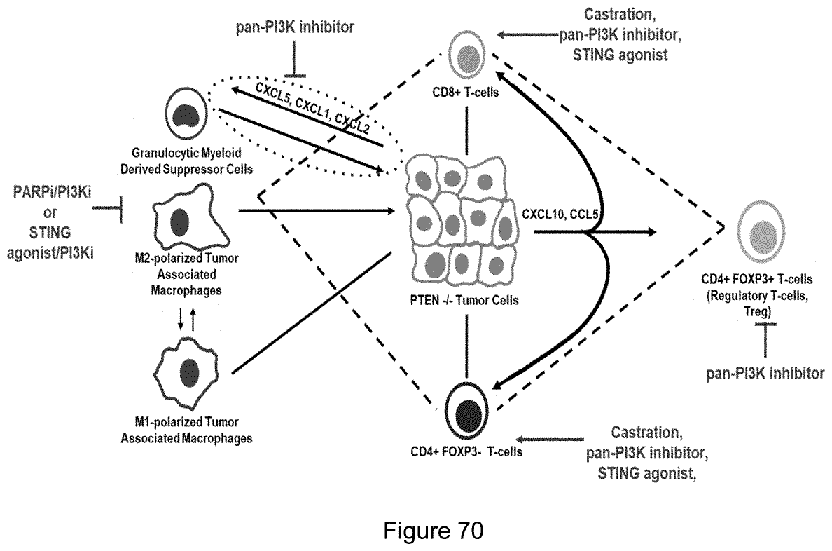

PTEN-deficient PCs have suppressed c-GAS/STING pathway activation via multiple mechanisms, which underlies their immunosuppressive TME. To create the inflammatory immune infiltrate of effector CD4 and CD8 T-cells required for an anti-tumor immune response in PTEN-deficient PC, it is necessary to reinvigorate the c-GAS/STING pathway within TME myeloid suppressive cells, regardless of underlying mechanism. Even with an inflammatory TME, CD4 and/or CD8 T-cells will typically need to be activated to realize a maximal anti-tumor response. These studies elucidate approaches for generating an inflammatory immune TME that could be responsive to PD1 and/or CTLA4 checkpoint inhibitors, with a focus on STING agonists and PI3Ki (FIG. 70). The studies are designed based on the premise that a combination of STING agonist (not PARPi) with PI3Ki can overcome the immunosuppressive TME of PTEN deficient PC, resulting in enhanced anti-tumor responses and ICB sensitization. The availability of pharmacologic STING agonists provides an opportunity to investigate this premise. STING agonist BMS-986301 is evaluated in preclinical PC models. It is recognized that intratumoral STING agonists have not shown robust anti-tumor efficacy in clinical trials, largely related to an inability to generate a systemic immune response beyond the locally injected site. BMS-986301 is a systemic STING agonist, thus abrogating the dependency on an abscopal effect to elicit systemic anti-tumor activity.

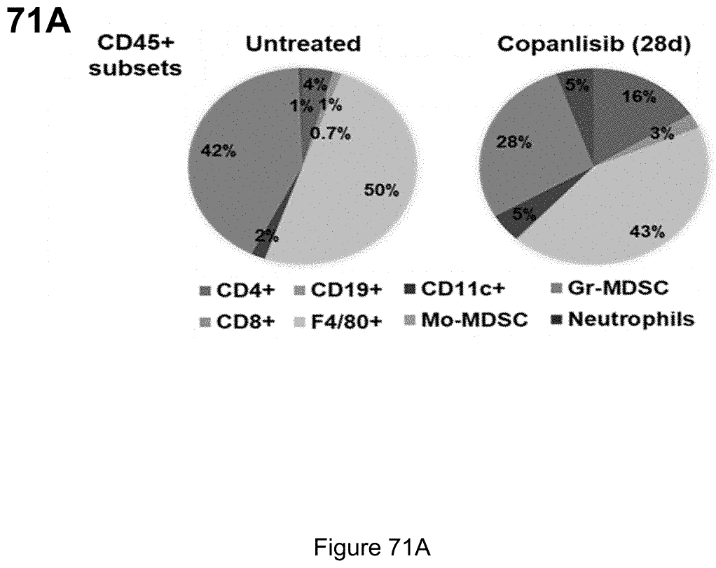

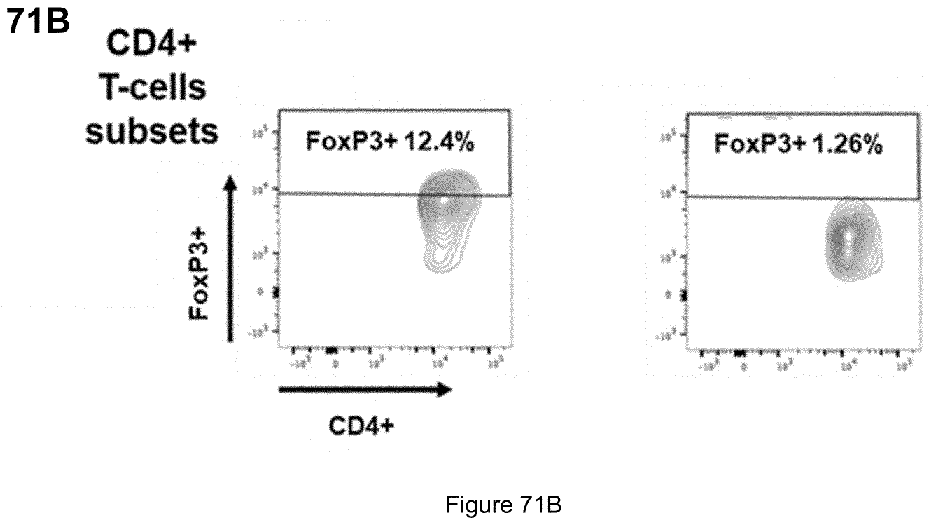

Consistent with the above, it was demonstrated that treatment with DMXAA, a mouse STING agonist, resulted in myeloid reprogramming and Type I interferon production within the TME of Myc-CAP-PTEN knockout and PTEN/p53-deficient GEMM tumors, bypassing the requirement for DNA DSB-mediated STING activation within myeloid cells. As expected, the TILs exhibit markers of increased exhaustion, including decreased 4-1BB, decreased ICOS and increased PD-1, emphasizing the need for ICB to realize an anti-tumor effect. Furthermore, DMXAA treatment of PTEN/p53 GEMM mice resulted in decreased MDSC infiltration, and reduction in inhibitory VISTA and PD-L1 checkpoint expression on MDSCs, providing an additional mechanism through which direct STING pathway activation can create an inflammatory TME poised for activation through checkpoint inhibitor therapy. As discussed earlier, PI3K inhibition also has the potential for enhancing an inflammatory TME. Thus, changes in immune infiltrate and anti-tumor response elicited by copanlisib, a pan-PI3K inhibitor are evaluated in PTEN/p53-deficient GEMMs. A striking increase is observed in CD4 T cell infiltration, with concomitant depletion of Gr-MDSCs and Tregs (FIG. 71).

Early castration reprograms the TME via decreased MDSCs and increased T cell infiltration. This effect is leveraged by evaluating the therapeutic impact of single-agent rucaparib (PARPi), copanlisib (pan-PI3Ki), and BMS-986301 (systemic STING agonist; Bristol Myers Squibb International Immuno-Oncology collaboration) on the immune microenvironment of uncastrated (UC), degarelix-treated castrate-sensitive (CS), or castrate-resistant (CR) orthotopic Myc-CAP or B6-myc, and their corresponding PTEN null tumors. More specifically, uncastrated mice bearing syngeneic PTEN-proficient and PTEN-deficient Myc-CAP (STING^{lo})/B6-myc (STING^{hi}) prostate tumors and PTEN/p53-deficient GEMM tumors are treated with rucaparib or copanlisib by oral gavage, or BMS-986301 via intramuscular injection, when the tumors reach 400 mm³ in size. For CS in vivo experiments, mice with syngeneic tumors are castrated when the tumors reach 400 mm³, and concomitantly treated with the indicated single agents. For CR experiments, castrated mice are treated with the indicated drugs following the development of castration-resistance, when CR tumors re-grow to 400 mm³. The mice are treated for 4 weeks or 2500 mm³, at which point they are euthanized. The following drug combinations are tested in the UC, CS, and CR settings: 1) rucaparib/copanlisib; 2) BMS-986301/copanlisib; 3) BMS-986301/rucaparib. The indicated drugs as single agents and the combinations are also tested in PTEN/p53-deficient GEMM mice, which are de novo resistant to castration. The tumors in GEMMs are monitored by MRI, and mice euthanized when the tumors reach 2 cm in long-axis diameter. The systemic STING agonist in combination with pan-PI3K inhibitor can overcome myeloid immunosuppression via MDSC depletion and enhance T cell infiltration and activation, resulting in potent anti-tumor immune responses, which are enhanced by degarelix in the CS disease state. Tumor sections at the time of euthanasia are harvested for western blot analysis, IHC, flow cytometry, and bulk-RNA-seq. Multidimensional analysis is carried out to interrogate drug target modulation, immune infiltrate composition, c-GAS/STING pathway activation within tumor cells and immune cell subsets, and discovery of response/resistance biomarkers. Longitudinal tumor growth data is analyzed in collaboration. Taken together, these studies identify rational IO partners to combine with ICB in PTEN-deficient PC.

Evaluating the combinatorial therapeutic impact of STING agonist/PI3Ki combinations on sensitization of PTEN-deficient syngeneic and GE W PC models to ICB.

STING agonist/PI3Ki combinations are expected to enhance an inflammatory TME, but are not necessarily expected to be sufficient for an anti-tumor response. A STING agonist/PI3Ki-induced STING pathway activation within the TME could sensitize PTEN-deficient PC to ICB. The anti-tumor efficacy of copanlisib (pan-PI3Ki) is evaluated in combination with BMS-986301 (STING agonist) for sensitization of PTEN-deficient syngeneic and GEMM models of PC to ICB, within the context of UC, CS and CR disease states. The impact of sequential (A→B or B→A) vs. concomitant administration of combinations with ICB administration on therapeutic efficacy is also assessed. The immune profiling, anti-tumor efficacy analysis, and endpoints for analysis are performed as described above.

Alternative approaches. The subcutaneous models are limited with respect to providing an autochthonous TME and the injections of cell lines subcutaneously can alter the inflammatory milieu within the tumor. If biologically meaningful differences in anti-tumor responses are not observed within the subcutaneous context, isogenic Myc-CAP and B6-myc experiments in orthotopic models and corresponding transgenic c-myc-driven GEMMs can also be carried out. Because recent data have shown that chronic tumor cell intrinsic c-GAS/STING can also provide a pro-tumorigenic signal potentially promoting metastasis, restoration of tumor cell intrinsic STING expression could enhance anti-tumor responses following STING agonist treatment. If tumor cell-intrinsic STING signaling enhances pro-tumorigenic inflammation, then restoration of STING expression within tumor cells can potentially attenuate the anti-tumor response following systemic STING agonist treatment. On the other hand, if tumor cell-intrinsic STING signaling enhances anti-tumor immune responses, then restoration of STING expression within tumor cells can potentially enhance anti-tumor immune responses and efficacy of STING agonists. It is also conceivable that acute vs. chronic c-GAS/STING signaling have opposing effects on tumor immune control, so pulsatile vs. continuous STING agonist-based combination therapies in murine PC models are also explored.

Biostatistical analysis. Therapeutic impact of multiple treatment regimens is analyzed, both individually and in combination (either sequentially or concomitantly) in several mouse models. Tumor size is measured over time, and data are analyzed using generalized linear mixed models with repeated measures. Continuous biomarkers, including immunoprofiling, T-cell diversity, and RNA profiling that are measured in tumors after euthanasia are compared using linear regression models with treatment group as the predictor, followed by post hoc pairwise t-tests if global differences are observed. Power considerations: For any pairwise comparison of independent mice on different treatments at a given time point, a sample size of n=10 mice in each group affords 80% power with 5% two-sided Type I error rate to detect a standardized effect size of Δ=1.32 at the end of treatment, an effect size that is smaller than that observed in data to date (e.g. Δ=3 in FIG. 68). Power is enhanced during longitudinal statistical modeling, due to repeated measures of outcomes within each mouse over time.

Analyzing Changes in Tumor Immune Microenvironment within Metastatic Biopsy Samples from Two Investigator-Initiated Trials Testing PD1-Based 10 Combination Therapies in mCRPC.

To complement the mechanistic and murine therapeutic studies outlined above, an investigator-initiated co-clinical trial of PD-1 antibody (nivolumab) with PARPi (rucaparib) in mCRPC patients was conducted. STING agonist-based (not PARPi-based) IO combinations, are likely to sensitize PTEN-deficient mCRPC patients to ICB. An investigator-initiated clinical trial of PD-1 antibody (nivolumab) with STING agonist (BMS-986301) and PI3Ki (copanlisib) is also conducted.

Analyzing metastatic TME changes in mCRPC biopsy samples from a Phase Ib/II IIT of PD-1 antibody (nivolumab) with PARPi (rucaparib).

A phase 1b IO combination trial of rucaparib with nivolumab in mCRPC patients (IRB18-0154, NCT03572478), which enrolled 12 patients, was conducted. Patients received pre- and on-treatment metastatic biopsies at 4 weeks. Consistent with newly generated preclinical data, a lack of anti-tumor efficacy was observed with the rucaparib/nivolumab combination, which was corroborated by a larger trial. The study was therefore terminated.

The collected metastatic specimens from this trial (6-7 cores/biopsy, approximately 120 samples total) are utilized to elucidate the mechanistic basis for the lack of anti-tumor response. Multidimensional “omics” data are integrated to decipher changes in the tumor immune microenvironment following treatment. Correlative studies are approached in two groups. In Group A, bulk RNAseq analysis is performed, as well as multiplex immunofluorescence (IF) on formalin-fixed paraffin embedded (FFPE) and zinc-fixed paraffin embedded tissue specimens. In Group B, flow cytometry and single cell RNA sequencing (sc-RNAseq) are conducted on metastatic core biopsies and bone marrow aspirates (obtained from bone metastases). In addition, transparent tissue tomography (T3) or 3D imaging on tissue cores is performed. Peripheral blood mononuclear cells (PBMCs) and serum was collected at baseline and regular intervals. The following studies are listed in order of priority, recognizing that not all studies are conducted with the limited samples obtained:

Bulk RNA-seq/Nanostring® to perform gene set enrichment and T-cell inflamed/non-inflamed gene signature analysis (Group A, core biopsy). RNA is isolated using an RNeasy® kit (QIAGEN®, Venlo, Netherlands), and sequenced by Nanostring®. The expression profile of 770 genes included in the PanCancer Immune Profiling Panel (Nanostring Technologies®) and 30 candidate genes associated with immune resistance described in a previous study (Chen, L., et al., CD38-Mediated Immunosuppression as a Mechanism of Tumor Cell Escape from PD-1/PD-L1 Blockade. Cancer Discov, 2018. 8(9): p. 1156-1175) are determined for each sample using the Nanostring nCounter® analysis system. Normalization of the raw Nanostring® data is conducted using the expression of 40 reference genes by nSolver™ Analysis Software v1.1. The tumor inflammation signature score is dichotomized as inflamed versus non-inflamed tumor, as per prior definitions (Ayers, M., et al., IFN-gamma-related mRNA profile predicts clinical response to PD-1 blockade. J Clin Invest, 2017. 127(8): p. 2930-2940).

T cell clonality analysis (Group A, core biopsy). The T-cell infiltrate is further characterized by conducting NGS of the complementarity determining regions (CDRs) of the T cell receptor variable beta (TCR Vβ) region. Increased T cell diversity is a measure of ‘antigen-spread’, and potentially enhanced anti-tumor recognition, and enhanced clinical responses. Clonality of tumor-infiltrating T cells is evaluated using TCR β-chain sequencing. Similar studies are conducted using CD4 and CD8 T cells that are isolated from peripheral blood samples, to assess for differential changes in clonal expansion between the periphery and the TME.

Multiplex IHC/IF analysis (Group A, core biopsy). Formalin- and zinc-fixed core biopsies are utilized for tumor- and immune-marker based IHC, respectively. Bone core biopsies have been decalcified. Tissue based cyclic immunofluorescence (IF) (t-CyCIF) is performed, which simultaneously detects multiple targets of interest in a single tissue section and enables spatial characterization of the TME. 5-μm thick sections of formalin fixed paraffin embedded tissue are deparaffinized, stained by hematoxylin for 1 min and scanned using RareCyte CyteFinder® system. Multiplexed IF microscopy for myeloid and lymphoid markers (CD45, CD8, CD3, Cd68, PD1, PDL1, RORgt, Tbet, GATA3, FoxP3, p16, CD56, CD20, tryptase, CD83, CD163, CD66b, MHC-II, CSF1R, DC-SIGN, Nuclei), consisting of iterative cycles of staining, scanning, and antibody-fluorophore bleaching are performed. Image analysis is done using ImageJ, Matlab®, Ashlar and BaSiC to differentiate individual cell populations in an unsupervised clustering manner.

Flow cytometry analysis (Group B, bone marrow aspirate or core #4, PBMC). For bone metastatic biopsies, bone marrow aspirate was collected and aliquoted at −80° C. The cell pellet is resuspended in a monolayer (approximately 1.5 mL), and a 400 μL aliquot is utilized for flow cytometry analysis. For tissue cores (bone and non-bone), biopsy chunks are gently dissociated in RPMI media and then centrifuged to obtain a cell pellet. Residual RBCs are removed by ACK treatment, following which cells are counted and then frozen in freezing mixture for downstream flow analysis. In addition, PBMCs have also been collected to conduct an unbiased comparative evaluation of immune populations within the TME and the periphery, and to yield data as to whether treatment results in an increased infiltration of inflammatory immune cells into the tumor. More specifically, samples are stained in replicates in 96-well round bottom microtiter plates (Corning®, Cambridge, Mass.) with antibodies including, but not limited to, CD3, CD4, CD8, CD11b, CD11c, CD14, CD16, CD19, CD33, CD45, CD56, CD66b, CD209), various markers of immune activation or inhibition (such as 4-1BB, PD-1, or PD-L1), and intracellular markers such as Foxp3. The data collected is analyzed by conventional flow cytometry software such as FlowJo® and SPADE.

sc-RNA-seq (Group B, core biopsy). Cryopreserved biopsies are individually dissociated (Singh, M., et al., High-throughput targeted long-read single cell sequencing reveals the clonal and transcriptional landscape of lymphocytes. Nat Commun, 2019. 10(1): p. 3120). CITE-Seq is used to profile the transcriptome and 130 cell surface antigens of ˜4000 cells each. Cells are partitioned on the Chromium controller from 10× Genomics®. Data is processed using 10× Genomics bioinformatics processing pipeline, CellRanger, coupled with state-of-the-art methods, such as Seurat (Singh, M., et al., High-throughput targeted long-read single cell sequencing reveals the clonal and transcriptional landscape of lymphocytes. Nat Commun, 2019. 10(1): p. 3120). Repertoire and Gene Expression by Sequencing (RAGE-Seq), a method combining targeted cDNA capture and long-read Nanopore sequencing of T-cell-receptor (TCR) and B-cell-receptor (BCR) mRNA transcripts with short-read transcriptome profiling of single cell libraries, is also applied. Tissue sections are analyzed using two methods: 1) The CODEX multiplex IF platform from Akoya Biosciences®. 2) The GEoMx® platform from Nanostring®, which allows spatial mapping of hundreds of mRNA and/or proteins.

Multiplex T3 analysis (Group B, core biopsy). Published methods are utilized (Lee, S. S., V. P. Bindokas, and S. J. Kron, Multiplex three-dimensional optical mapping of tumor immune microenvironment. Sci Rep, 2017. 7(1): p. 17031). The biopsy was immediately fixed with 2% paraformaldehyde and for bone samples, decalcification was performed. The biopsy sample is placed in pre-casted 2% agarose mold and incubated with 1 mL antibody cocktails for 18 h at 4° C. The immunostained biopsy is mounted between coverslips in 100 w/v % D-fructose solutions, scanned at 10× objective of Leica® TCS SP8 confocal laser scanning microscope, and 3D reconstruction of images done using Fiji®, Huygens® Pro and Imaris® software. The 3D distribution of cancer and immune cells is analyzed with respect to CD31+ vascular cells using 3D-skeleton and 3D-Euclidean distance map. Tumor cells are delineated using PSA, and immune cells are stained for CD4, CD8 and CD33.

Analyzing metastatic tumor immune microenvironment changes in mCRPC biopsy samples from Phase Ib/II IIT of PD-1 antibody (nivolumab) with STING agonist (BMS-986301) and PI3Ki (copanlisib).

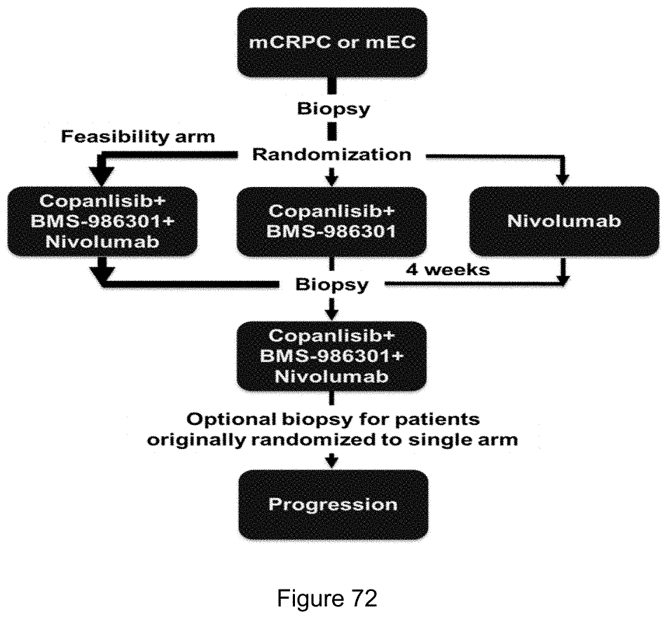

Given the failure of the PARPi/PD-1 combination, these studies aim to develop a next-generation clinical trial of STING agonist/PI3Ki combination and reverse myeloid immunosuppresion within the TME, sensitizing both PTEN-proficient and PTEN-deficient mCRPC to ICB. A Phase IB/II investigator-initiated triple combination clinical trial is conducted of BMS-986301/copanlisib/nivolumab in mCRPC patients (FIG. 72). The results of this trial are directly compared with the data obtained from the Phase I trial of BMS-986301/ipilimumab/nivolumab in advanced cancers, which is enrolling patients (IRB18-1829, NCT03956680). Patients with mCRPC undergo a 4-week lead-in period with the triple combination to confirm safety/feasibility. Additional patients are then randomized to receive BMS-986301/copanlisib or nivolumab alone during the 4-week lead-in period. All patients receive the triple combination therapy following the 4-week lead-in phase, until disease progression. Metastatic lesions are biopsied pre-treatment, following the 4-week lead-in period, as well as an optional biopsy at the time of disease progression. Metastatic biopsies are collected and analyzed, as described herein, and patients are stratified retrospectively by PTEN status.

Alternative approaches. If the cyclic immunofluoresence technology does not yield satisfactory results, standard IHC for CD4, CD8, FoxP3, 4-1BB, PD1, and other immune markers can be conducted. Multiple cores and correlative studies are conducted and it is recognized that the patient-derived material is limited. The studies are prioritized to assure that the primary phase 2 endpoint of determining whether IO triple combination enhances T cell infiltration relative to doublet or ICB alone can be addressed.

Biostatistical analysis. These studies involve correlative studies using mCRPC biopsies from a completed Phase 1b/Phase 2 clinical trial which was stopped early with n=12 due to lack of efficacy. Tumors are categorized as inflamed or non-inflamed based on tumor inflammation score (Ayers, M., et al., IFN-gamma-related mRNA profile predicts clinical response to PD-1 blockade. J Clin Invest, 2017. 127(8): p. 2930-2940), and differences in gene expression are evaluated using RNA-Seq data. Biomarkers evaluated through multiple IF, flow cytometry/CYTOF data and multiplex T3 data is compared between inflamed and non-inflamed tumors using two sample t-tests or Wilcoxon ranksum test. A Phase IB/II investigator-initiated clinical trial is conducted in mCRPC patients, in which patients are randomized to three treatment arms: Nivolumab (Arm A), BMS-986301/Copanlisib (Arm B), or Nivolumab/BMS-986301/Copanlisib (Arm C), and Arm A and B patients cross-over to Arm C following 4 weeks of treatment. The primary endpoint is to assess changes in T-cell inflammation in Arm B vs. Arm A, and Arm C vs. Arm A. With n=20 subjects per arm, there is 80% power with two-sided Bonferroni-corrected α=0.025 (overall Type I error rate α=0.05) to detect a difference between 10% vs. 60% of subjects with inflamed tumors in Arms B vs. Arm A and Arm C vs. Arm A, based on Fisher's exact test. Correlative studies are analyzed, as described herein.

Having described the invention in detail and by reference to specific aspects and/or embodiments thereof, it will be apparent that modifications and variations are possible without departing from the scope of the invention defined in the appended claims. More specifically, although some aspects of the present invention may be identified herein as particularly advantageous, it is contemplated that the present invention is not limited to these particular aspects of the invention. All references cited herein are incorporated by reference for all purposes.

What is claimed is:

1. A method of treating cancer in a subject, comprising:
a) determining a PTEN status of a tumor of the subject; and
b) administering a PARP inhibitor and a PI3Kinase inhibitor to the subject if the status of the tumor is determined to be PTEN-proficient.

2. The method of claim 1, wherein the cancer is prostate cancer.

3. The method of claim 2 further comprising c) administering androgen deprivation therapy to the subject.

4. The method of claim 1, wherein determining the PTEN status comprises taking a tissue biopsy from the tumor of the subject.

5. The method of claim 1, wherein determining the PTEN status comprises next generation sequencing and/or immunohistochemistry.

6. The method of claim 1 further comprising c) administering an immune checkpoint blockade-targeting therapy to the subject.

7. A method of treating cancer in a subject, comprising:
a) determining a PTEN status of a tumor of the subject; and
b) administering a STING agonist to the subject if the status of the tumor is determined to be PTEN-deficient.

8. The method of claim 7, wherein the tumor is PTEN-deficient if one or more cells within the tumor is determined to be PTEN-deficient

9. The method of claim 7, wherein the cancer is prostate cancer.

10. The method of claim 9 further comprising step c) administering androgen deprivation therapy to the subject.

11. The method of claim 9, wherein the subject is non-responsive to androgen deprivation therapy.

12. The method of claim 7, wherein the cancer is ovarian cancer.

13. The method of claim 7, wherein the ovarian cancer is endometrial cancer.

14. The method of claim 12 further comprising step c) administering chemotherapy to the subject.

15. The method of claim 7, wherein determining the PTEN status comprises taking a tissue biopsy from the tumor of the subject.

16. The method of claim 7, wherein determining the PTEN status comprises next generation sequencing and/or immunohistochemistry.

17. The method of claim 7, wherein the STING agonist is BMS-986301.

18. The method of claim 7 further comprising step c) administering a PI3Kinase inhibitor to the subject.

19. A method of treating cancer in a subject, comprising:
a) detecting the presence of DNA double-strand break fragments associated with an exosome or a microvesicle in a tumor microenvironment; and
b) administering an immune checkpoint blockade-targeting therapy and a PI3Kinase inhibitor to the subject if DNA double-strand break fragments associated with an exosome or a microvesicle in the tumor microenvironment are detected.

20. The method of claim 19, wherein detecting the presence of DNA double-strand break fragments comprises taking a blood sample from the subject.
